(19) United States
(12) Patent Application Publication
(10) Pub. No.: US 2024/0309046 A1
(43) Pub. Date: Sep. 19, 2024

(54) COMPSTATIN ANALOGUES AND THEIR MEDICAL USES

(71) Applicant: ZP SPV 3 K/S, Søborg (DK)

(72) Inventors: Anne Pernille Tofteng SHELTON, Søborg (DK); Jacob Ulrik FOG, Søborg (DK); Jens Kvist MADSEN, Søborg (DK)

(21) Appl. No.: 18/601,031

(22) Filed: Mar. 11, 2024

Related U.S. Application Data
(63) Continuation of application No. 16/976,439, filed on Aug. 27, 2020, now Pat. No. 11,965,039.

(30) Foreign Application Priority Data
Feb. 27, 2018 (EP) 18158834.4
Dec. 20, 2018 (EP) 18214949.2

(51) Int. Cl. C07K 7/08; A61K 38/00; A61K 47/54; A61K 47/65

(52) U.S. Cl. CPC C07K 7/08; A61K 47/542; A61K 47/545; A61K 47/65; A61K 38/00

(57) ABSTRACT

Compstatin analogues having improved binding and complement-inhibiting activity as compared to the 13 amino acid compstatin peptide (ICVVQDWGHHRCT (cyclic C2-C12)) are described, in particular compstatin analogues that additionally possess useful physicochemical properties, such as increased solubility. These analogues include variants with an isoleucine residue at position 3 in place of the wild type valine residue, which provides compstatin peptides with improved binding and complement-inhibiting activity and also enables the introduction of other modifications, for example modifications that are capable of increasing solubility, such as the introduction of charged or polar amino acids at position 9 and/or the introduction of N-and/or C-terminal sequences.

### SEQUENCE LISTING

The instant application contains a Sequence Listing which has been submitted electronically in XML format and is hereby incorporated by reference in its entirety. Said XML copy, created on Mar. 20, 2024, is named 51574-002004_Sequence_Listing_3_20_24 and is 836,536 bytes in size.

### FIELD OF THE INVENTION

The present invention relates to inhibiting activation of the complement cascade in the body, and more particularly to compstatin analogues that are capable of binding to C3 protein and inhibiting complement activation. The present invention also relates to the medical uses of the compstatin analogues, in particular for the treatment of conditions characterized by unwanted activation of the complement cascade, such as autoimmune and inflammatory diseases.

### BACKGROUND OF THE INVENTION

The human complement system is a powerful player in the defense against pathogenic organisms and the mediation of immune responses. Complement can be activated through three different pathways: the classical, lectin and alternative pathways. The major activation event that is shared by all three pathways is the proteolytic cleavage of the central protein of the complement system, C3, into its activation products C3a and C3b by C3 convertases. Generation of these fragments leads to the opsonization of pathogenic cells by C3b and iC3b, a process that renders them susceptible to phagocytosis or clearance, and to the activation of immune cells through an interaction with complement receptors (Markiewski & Lambris, 2007, Am. J. Pathol., 171: 715-727). Deposition of C3b on target cells also induces the formation of new convertase complexes and thereby initiates a self-amplification loop. An ensemble of plasma and cell surface-bound proteins carefully regulates complement activation to prevent host cells from self-attack by the complement cascade. However, excessive activation or inappropriate regulation of complement can lead to a number of pathologic conditions, ranging from autoimmune to inflammatory diseases (Holers, 2003, Clin. Immunol., 107: 140-51; Markiewski & Lambris, 2007, supra; Ricklin & Lambris, 2007, Nat. Biotechnol., 25: 1265-75; Sahu et al., 2000, J. Immunol., 165: 2491-9). The development of therapeutic complement inhibitors is therefore highly desirable. In this context, C3 and C3b have emerged as promising targets because their central role in the cascade allows for the simultaneous inhibition of the initiation, amplification, and downstream activation of complement (Ricklin & Lambris, 2007, supra).

Compstatin was first identified as a 27 amino acid peptide and was the first non-host-derived complement inhibitor that was shown to be capable of blocking all three activation pathways (Sahu et al., 1996, J. Immunol., 157: 884-91; U.S. Pat. No. 6,319,897). It has been shown that it is possible to truncate compstatin without loss of activity to a 13 amino acid peptide. However, attempts to further truncate this peptide led to loss of activity. The sequence of the 13 amino acid truncated (or “core”) compstatin peptide is Ile¹-Cys²-Val³-Val⁴-Gln⁵-Asp⁶-Trp⁷-Gly⁸-His⁹-His¹⁰-Arg¹¹-Cys¹²-Thr¹³-NH₂ (SEQ ID NO: 1), where Cys² and Cys¹² are disulfide bonded. This cyclic tridecapeptide binds to C3 (and fragments of C3), thereby inhibiting the activation of the downstream complement cascade and preventing the cleavage of native C3 by the C3 convertases. Its inhibitory efficacy was confirmed by a series of studies using experimental models that pointed to its potential as a therapeutic agent (Fiane et al, 1999a, Xenotransplantation, 6: 52-65; Fiane et al,, 1999b, Transplant Proc., 31:934-935; Nilsson et al., 1998, Blood, 92: 1661-1667; Ricklin & Lambris, 2008, Adv. Exp. Med. Biol., 632: 273-292; Schmidt et al., 2003, J. Biomed. Mater. Res., A66: 491-499; Soulika et al., 2000, Clin. Immunol., 96: 212-221).

Progressive optimization of the 13 amino acid compstatin peptide has led to analogues with improved biological activity (Ricklin & Lambris, 2008, supra; WO 2004/026328; WO 2007/062249, WO 2013/036778, WO 2014/100407).

Earlier structure-activity studies have identified the cyclic nature of the compstatin peptide and the presence of both a β-turn and hydrophobic cluster as key features of the molecule (Morikis et al., 1998, Protein Sci., 7: 619-627; WO 99/13899; Morikis et al., 2002, J. Biol. Chem., 277:14942-14953; Ricklin & Lambris, 2008, supra). Hydrophobic residues at positions 4 and 7 were found to be of particular importance, and their modification with unnatural amino acids generated an analogue with 264-fold improved activity over the original compstatin peptide (Katragadda et al., 2006, J. Med. Chem., 49: 4616-4622; WO 2007/062249). Further attempts to optimize compstatin for use in the treatment of eye disorders are described in WO 2007/044668.

While previous optimization steps have been based on combinatorial screening studies, solution structures, and computational models (Chiu et al., 2008, Chem. Biol. Drug Des., 72: 249-256; Mulakala et al., 2007, Bioorg. Med. Chem., 15: 1638-1644; Ricklin & Lambris, 2008, supra), the publication of a co-crystal structure of compstatin complexed with the complement fragment C3c (Janssen et al., 2007, J. Biol. Chem., 282: 29241-29247; WO 2008/153963) provided a basis for initiating rational optimization. The crystal structure revealed a shallow binding site at the interface of macroglobulin (MG) domains 4 and 5 of C3c and showed that 9 of the 13 amino acids were directly involved in the binding, either through hydrogen bonds or hydrophobic interactions. As compared to the structure of the compstatin peptide in solution (Morikis et al., 1998, supra), the bound form of compstatin experienced a conformational change, with a shift in the location of the β-turn from residues 5-8 to 8-11 (Janssen et al., 2007, supra; WO 2008/153963).

In view of its therapeutic potential in AMD, C3G, PNH and other diseases, it remains a problem in the art to further optimize compstatin analogues, for example to achieve an even greater activity and/or to modulate pharmacokinetic properties, such as increased half-life in vivo and/or physicochemical properties such as increased solubility.

### SUMMARY OF THE INVENTION

Broadly, the present invention is based on work to develop a new family of compstatin analogues having improved binding and complement-inhibiting activity as compared to the 13 amino acid compstatin peptide (ICVVQDWGHHRCT (cyclic C2-C12)) (SEQ ID NO: 1). In some cases, these compstatin analogues additionally possess useful physicochemical properties, such as increased solubility. In particular, the present inventors found that introducing an isoleucine residue at position 3 in place of the wild type valine residue led to compstatin peptides with improved binding and complement-inhibiting activity. The present inventors further discovered that the introduction of isoleucine at position 3 enables the introduction of other modifications, for example modifications that are capable of increasing solubility, such as the introduction of glutamic acid at position 6, particular charged or polar amino acids at position 9, and/or the introduction of N-and/or C-terminal sequences. Example of such additional modifications include the replacement of Ile at position 1 with Tyr, Phe or Sar, replacement of Val at position 4 with Trp, a Trp analogue (as described herein); replacement of Asp in position 6 with Glu; replacement of His at position 9 with Ala, Glu, Asp, Lys, Ser or Arg; replacement of Arg at position 11 with Ser; replacement of Thr at position 13 with Ser, Glu, Sar or Ile. Preferred compstatin peptides including one or more of these modifications have improved solubility, for example as compared to the 13 amino acid compstatin peptide (ICVVQDWGHHRCT (cyclic C2-C12)) (SEQ ID NO: 1). Further examples of these compstatin peptides combine modification at position 9 with extensions to the N-terminal and/or C-terminus of the peptide.

Accordingly, the present invention provides a compstatin analogue represented by the formula:

(Formula I)
(SEQ ID NO: 2)
Y1-R1-X1-C-I-X4-Q-X6-W-X8-X9-H-X11-C-X13-R2-Y2

wherein:
Y1 is hydrogen, acetyl or a lipophilic group Φ;
X1 is I, Y, F or Sar;
X4 is W, F, V, Y, 1-Me-Trp, D-Trp, N-Me-Trp, 1-For-Trp, 1-Nal, 2-Nal, 5-Me-Trp, Bpa or 2-Igl;
X6 is E, K or D;
X8 is G or Sar;
X9 is H, A, E, D, K, R or S;
X11 is R, S or K;
X13 is T, S, E, F, H, K, Sar, G, I, D, N-Me-Ile or N-Me-Thr;
Y2 is NH₂, OH or a lipophilic group Φ;
R1 is absent or is a sequence of 1 to 6 amino acid residues selected from A, E, G, L, K, F, P, S, T, W, Y, R, V, Sar, εLys, γGlu, βAsp, or βAla, or a corresponding D form thereof; or Peg3, Peg4, or 8-aminooctanoyl, or derivatives thereof; and
R2 is absent or is a sequence of 1 to 8 amino acid residues selected from A, E, G, L, K, F, P, S, T, W, Y, R, V, Sar, εLys, γGlu, βAsp, or βAla, or a corresponding D form thereof; or Peg3, Peg4, or 8-aminooctanoyl, or derivatives thereof;
wherein the compstatin analogue has a disulphide bond between the cysteine residues at positions 2 and 12;
and wherein the compstatin analogue optionally has a lipophilic group Φ covalently linked to the side chain of one or more amino acid residues;
or a pharmaceutically acceptable salt and/or solvate thereof.

In some embodiments, X11 is R or S.

In some embodiments, if a lipophilic group Φ is linked to the side chain of an amino acid residue, that residue is the residue at position X1, X11 or X13, or is a residue in R1 or R2. It may be a lysine residue. For example, it may be a lysine residue at position X11 or X13, or a lysine residue in R1 or R2.

In some embodiments, Y1 is hydrogen or acetyl.

In some embodiments, Y2 is NH₂ or OH.

In some embodiments, the compstatin analogue comprises at least one lipophilic group Φ, e.g. exactly one lipophilic group Φ.

In some embodiments, the compstatin analogue does not comprise a lipophilic group Φ.

The present invention further provides a compstatin analogue represented by the formula:

(Formula II)
(SEQ ID NO: 3)
Y1-R1-X1-C-I-X4-Q-X6-W-X8-X9-H-X11-C-X13-R2-Y2

wherein:
Y1 is hydrogen, acetyl, or a lipophilic group Φ;
X1 is I, Y, F or Sar;
X4 is W, V, Y, 2-Nal, 1-Nal or 1-Me-Trp;
X6 is E or D;
X8 is G or Sar;
X9 is A, E, D, K or S;
X11 is R, S or K;
X13 is T, S, E, I, Sar, K, G or N-Me-Ile;
Y2 is NH₂, OH or a lipophilic group Φ;
R1 is absent or is a sequence of 1 to 6 amino acid residues selected from A, E, G, L, K, F, P, S, T, W, Y, R, V, Sar, εLys, γGlu, βAsp, or βAla, or a corresponding D form thereof, or Peg3, Peg4, or 8-aminooctanoyl, or derivatives thereof; and
R2 is absent or is a sequence of 1 to 8 amino acid residues selected from A, E, G, L, K, F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein the compstatin analogue has a disulphide bond between the cysteine residues at positions 2 and 12;
and wherein the compstatin analogue optionally has a lipophilic group Φ covalently linked to the side chain of one or more amino acids;
or a pharmaceutically acceptable salt and/or solvate thereof.

In some embodiments, X11 is R or S.

In some embodiments, if a lipophilic group Φ is linked to the side chain of an amino acid residue, that residue is the residue at position X1, X11 or X13, or is a residue in R1 or R2. It may be a lysine residue. For example, it may be a lysine residue at position X13, or a lysine residue in R1 or R2.

In some embodiments, Y1 is hydrogen or acetyl.

In some embodiments, Y2 is NH₂ or OH.

In some embodiments, the compstatin analogue comprises at least one lipophilic group Φ, e.g. exactly one lipophilic group Φ.

In some embodiments of this formula, the compstatin analogue does not comprise a lipophilic group Φ.

The present invention further provides a compstatin analogue represented by the formula:

(Formula III)
(SEQ ID NO: 4)
Y1-R1-X1-C-I-X4-Q-X6-W-G-X9-H-X11-C-X13-R2-Y2

wherein:
Y1 is hydrogen, acetyl or a lipophilic group Φ;
X1 is I, Y, F or Sar;
X4 is W, V, Y, 1-Nal, 2-Nal or 1-Me-Trp;
X6 is E or D;
X9 is A, E, D, K or S;
X11 is R, S or K;
X13 is T, I, S, E, K or Sar;
Y2 is NH₂, OH or a lipophilic group Φ;
R1 is absent or is a sequence of 1 to 6 amino acid residues selected from A, E, G, L, K, F, P, S, T, W, Y, R, V, Sar, εLys, γGlu, βAsp, or βAla, or a corresponding D form thereof, or Peg3, Peg4, or 8-aminooctanoyl, or derivatives thereof; and
R2 is absent or is a sequence of 1 to 8 amino acid residues selected from A, E, G, L, K, F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein the compstatin analogue has a disulphide bond between the cysteine residues at positions 2 and 12;
and wherein the compstatin analogue optionally has a lipophilic group Φ covalently linked to the side chain of one or more amino acids;
or a pharmaceutically acceptable salt and/or solvate thereof.

In some embodiments, X11 is R or S.

In some embodiments, if a lipophilic group Φ is linked to the side chain of an amino acid residue, that residue is the residue at position X1, X11 or X13, or is a residue in R1 or R2. It may be a lysine residue. For example, it may be a lysine residue at position X11 or X13, or a lysine residue in R1 or R2.

In some embodiments, Y1 is hydrogen or acetyl.

In some embodiments, Y2 is NH₂ or OH.

In some embodiments, the compstatin analogue comprises at least one lipophilic group Φ, e.g. exactly one lipophilic group Φ.

In some embodiments of this formula, the compstatin analogue does not comprise a lipophilic group Φ.

The compstatin analogue may be represented by the formula:

(Formula IV)
(SEQ ID NO: 5)
Y1-R1-X1-C-I-X4-Q-X6-W-G-X9-H-R-C-X13-R2-Y2

wherein:
Y1 is hydrogen, acetyl or a lipophilic group Φ;
X1 is I, Y, F or Sar;
X4 is W, V, Y, 1-Nal, 2-Nal or 1-Me-Trp;
X6 is E or D;
X9 is A, E, D, K or S;
X13 is T, S, E or Sar;
Y2 is NH₂, OH or a lipophilic group ®;
R1 is absent or is a sequence of 1 to 6 amino acid residues selected from A, E, G, L, K, F, P, S, T, W, Y, R, V, Sar, εLys, γGlu, βAsp, or βAla, or a corresponding D form thereof, or Peg3, Peg4, or 8-aminooctanoyl, or derivatives thereof; and
R2 is absent or is a sequence of 1 to 8 amino acid residues selected from A, E, G, L, K, F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein the compstatin analogue has a disulphide bond between the cysteine residues at positions 2 and 12;
and wherein the compstatin analogue optionally has a lipophilic group Φ covalently linked to the side chain of one or more amino acids;
or a pharmaceutically acceptable salt and/or solvate thereof.

In some embodiments, if a lipophilic group Φ is linked to the side chain of an amino acid residue, that residue is the residue at position X1, X11 or X13, or is a residue in R1 or R2. It may be a lysine residue. For example, it may be a lysine residue at position X13, or a lysine residue in R1 or R2.

In some embodiments, Y1 is hydrogen or acetyl.

In some embodiments, Y2 is NH₂ or OH.

In some embodiments, the compstatin analogue comprises at least one lipophilic group Φ, e.g. exactly one lipophilic group Φ.

In some embodiments of this formula, the compstatin analogue does not comprise a lipophilic group Φ.

In some embodiments of the formulae above, X6 is D.

In one aspect, compstatin analogues which do not possess a lipophilic group Φ may be represented by the formula:

(Formula V)
(SEQ ID NO: 6)
Y1-R1-X1-C-I-X4-Q-X6-W-G-X9-H-R-C-X13-R2-Y2

wherein:
Y1 is hydrogen or acetyl;
X1 is Y or F;
X4 is W, Y, 1-Me-Trp;
X6 is E or D;
X9 is A, E or K;
X13 is T, E or Sar;
Y2 is NH₂ or OH;
R1 is absent or is a sequence of 1 to 6 amino acid residues selected from A, E, G, L, K, F, P, S, T, W, Y, R, V, Sar, εLys, γGlu, βAsp, or βAla, or a corresponding D form thereof, or Peg3, Peg4, or 8-aminooctanoyl, or derivatives thereof; and
R2 is absent or is a sequence of 1 to 6 amino acid residues selected from A, E, G, L, K, F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein the compstatin analogue has a disulphide bond between the cysteine residues at positions 2 and 12;
or a pharmaceutically acceptable salt and/or solvate thereof.

The compstatin analogue may be represented by the formula:

(Formula VI)
(SEQ ID NO: 7)
Y1-R1-X1-C-I-[1-Me-Trp]-Q-X6-W-G-E-H-R-C-X13-R2-Y2

wherein:
Y1 is hydrogen or acetyl;
X1 is Y or F;
X6 is E or D;
X13 is T, E or Sar;
Y2 is NH₂ or OH;
R1 is absent or is a sequence of 1 to 6 amino acid residues selected from A, E, G, L, K, F, P, S, T, W, Y, R, V, Sar, εLys, γGlu, βAsp, or βAla, or a corresponding D form thereof, or Peg3, Peg4, or 8-aminooctanoyl, or derivatives thereof; and
R2 is absent or is a sequence of 1 to 6 amino acid residues selected from A, E, G, L, K, F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein the compstatin analogue has a disulphide bond between the cysteine residues at positions 2 and 12;
or a pharmaceutically acceptable salt and/or solvate thereof.

In the formulae above X6 may be D. Alternatively it may be E.

In some embodiments, the compstatin analogue has the formula:

(Formula VII)
(SEQ ID NO: 8)
Y1-R1-X1-C-I-X4-Q-X6-W-X8-X9-H-X11-C-X13-R2-Y2

wherein:
Y1 is hydrogen, acetyl or a lipophilic group Φ;
X1 is I, Y, F or Sar;
X4 is W, V, 1-Me-Trp, 1-Nal or 2-Nal;
X6 is E, K or D;
X8 is G or Sar;
X9 is H, A, E, D, K, R or S;
X11 is R, S, K or K *;
X13 is T, S, E, Sar or N-Me-Ile;
Y2 is NH₂ or OH;
R1 and R2 may be as defined in any of the formulae above, or elsewhere in this specification. In some embodiments, R1 is absent or is a sequence of 1 to 6 amino acid residues selected from A, E, G, K, K*, S, Y, or a corresponding D form thereof; and/or R2 is absent or is a sequence of 1 to 8 amino acid residues selected from A, E, G, K, K*, P, S, Peg3, γGlu, 8-aminooctanoyl, or a corresponding D form thereof;
wherein * indicates that the amino acid residue bears a lipophilic group Φ covalently linked to its side chain.

It may be desirable that the compstatin analogue comprises at least one lipophilic group Φ, e.g. exactly one lipophilic group Φ. Alternatively, it may comprise no lipophilic group Φ.

In an alternative aspect, compstatin analogues which comprise a lipophilic group Φ may be represented by the formula:

(Formula VIII)
(SEQ ID NO: 9)
Y1-R1-X1-C-I-X4-Q-X6-W-X8-X9-H-X11-C-X13-R2-Y2

wherein:
Y1 is hydrogen, acetyl or a lipophilic group Φ;
X1 is I, Y, F or Sar;
X4 is W, V, Y, 2-Nal, 1-Nal or 1-Me-Trp;
X6 is E or D;
X8 is G or Sar;
X9 is A, E, D, K or S;
X11 is R, S or K *;
X13 is T, S, E, I, Sar, K, G or N-Me-Ile;
Y2 is NH₂, OH or a lipophilic group Φ;
R1 is absent or is a sequence of 1 to 6 amino acid residues selected from A, E, G, L, K, K*, F, P, S, T, W, Y, R, V or Sar, or a corresponding D form thereof;
R2 is absent or is a sequence of 1 to 8 amino acid residues selected from A, E, G, L, K, K*, F, P, S, T, W, Y, R, V Sar, εLys, γGlu, βAsp, or βAla, or a corresponding D form thereof; or Peg 3 or Peg4, or 8-aminooctanoyl, or derivatives thereof;
wherein * indicates that the amino acid residue bears a lipophilic group Φ covalently linked to its side chain;
wherein the compstatin analogue has a disulphide bond between the cysteine residues at positions 2 and 12;
and wherein the compstatin analogue comprises at least one lipophilic group Φ, e.g. exactly one lipophilic group Φ;
or a pharmaceutically acceptable salt and/or solvate thereof.

In some embodiments, Y2 is NH₂ or OH.

The compstatin analogue may be represented by the formula:

(Formula IX)
(SEQ ID NO: 10)
Y1-R1-X1-C-I-X4-Q-X6-W-G-X9-H-X11-C-X13-R2-Y2

wherein:
Y1 is hydrogen, acetyl, or a lipophilic group Φ;
X1 is I, Y, F or Sar;
X4 is W, V, Y, 1-Nal, 2-Nal or 1-Me-Trp;
X6 is E or D;
X9 is A, E, D, K or S;
X11 is R, S or K *;
X13 is T, I, S, E, Kor Sar;
Y2 is NH₂, OH or a lipophilic group Φ;
R1 is absent or is a sequence of 1 to 6 amino acid residues selected from A, E, G, L, K, K*, F, P, S, T, W, Y, R, V or Sar, or a corresponding D form thereof;
R2 is absent or is a sequence of 1 to 8 amino acid residues selected from A, E, G, L, K, K* F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein * indicates that the amino acid residue bears a lipophilic group Φ covalently attached to its side chain;
wherein the compstatin analogue has a disulphide bond between the cysteine residues at positions 2 and 12;
and wherein the compstatin analogue comprises at least one lipophilic group Φ, e.g. exactly one lipophilic group Φ;
or a pharmaceutically acceptable salt and/or solvate thereof.

In some embodiments, Y2 is NH₂ or OH.

The compstatin analogue may be represented by the formula:

(Formula X)
(SEQ ID NO: 11)
Y1-R1-X1-C-I-X4-Q-X6-W-G-X9-H-R-C-X13-R2-Y2

wherein:
Y1 is hydrogen, acetyl or a lipophilic group Φ;
X1 is I, Y, F or Sar;
X4 is W, V, 1-Nal, 2-Nal or 1-Me-Trp;
X6 is E or D;
X9 is A, E, D, K or S;
X13 is T, S, E or Sar;
Y2 is NH₂, OH or a lipophilic group Φ;
R1 is absent or is a sequence of 1 to 6 amino acid residues selected from A, E, G, L, K, K*, F, P, S, T, W, Y, R, V or Sar, or a corresponding D form thereof;
R2 is absent or is a sequence of 1 to 8 amino acid residues selected from A, E, G, L, K, K*, F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein * indicates that the amino acid residue bears a lipophilic group Φ covalently attached to its amino acid side chain;
wherein the compstatin analogue has a disulphide bond between the cysteine residues at positions 2 and 12;
and wherein the compstatin analogue comprises at least one lipophilic group Φ, e.g. exactly one lipophilic group Φ;
or a pharmaceutically acceptable salt and/or solvate thereof.

In some embodiments, Y2 is NH₂ or OH.

In any of the formulae above, X6 may be D. Alternatively X6 may be E.

In any of the formulae above, X1 may be Y. Alternatively X1 may be F.

In any of the formulae above, X13 may be Sar. Alternatively X13 may be T.

Additionally or alternatively, any of the frmulae above may comprise one of the following combinations of residues:
X4 is 1-Me-Trp and X9 is E.
X1 is F, X4 is 1-Me-Trp and X9 is E.
X4 is 1-Me-Trp, X9 is E and X13 is Sar.
X4 is 1-Me-Trp, X9 is E and X13 is T.
X4 is 1-Me-Trp, X6 is D, X9 is E and X13 is Sar.
X4 is 1-Me-Trp, X6 is E, X9 is E and X13 is Sar.
X4 is 1-Me-Trp, X6 is D, X9 is E and X13 is T.
X4 is 1-Me-Trp, X6 is E, X9 is E and X13 is T.

The compstatin analogue may be represented by the formula:

(Formula XI)
(SEQ ID NO: 12)
Y1-R1-X1-C-I-[1-Me-Trp]-Q-X6-W-G-E-H-R-C-X13-R2-Y2

wherein:
Y1 is hydrogen or acetyl;
X1 is Y or F;
X6 is E or D;
X13 is T, E or Sar;
Y2 is NH₂ or OH;
R1 is absent or is a sequence of 1 to 6 amino acid residues selected from A, E, G, L, K, K*, F, P, S, T, W, Y, R, V or Sar, or a corresponding D form thereof;
R2 is absent or is a sequence of 1 to 8 amino acid residues selected from A, E, G, L, K, K* F, P, S, T, W, Y, R, V, Sar, εLys, γGlu, βAsp, or βAla, or a corresponding D form thereof; or Peg3 or Peg4, or 8-aminooctanoyl, or derivatives thereof;
wherein * indicates that the amino acid residue bears a lipophilic group Φ covalently attached to its side chain;
wherein the compstatin analogue has a disulphide bond between the cysteine residues at positions 2 and 12;
and wherein the compstatin analogue comprises at least one lipophilic group Φ, e.g. exactly one lipophilic group Φ;
or a pharmaceutically acceptable salt and/or solvate thereof.

In some embodiments, the 13-mer peptide portion (X1-X13) of the compstatin analogue has a sequence selected from:

(SEQ ID NO: 13)
[Sar]C(1)|[1-Me-Trp]QDWGEHRC(1)[Sar];
(SEQ ID NO: 14)
[Sar]C(1)|[1-Me-Trp]QDWGEHRC(1)T;
(SEQ ID NO: 15)
[Sar]C(1)|[1-Me-Trp]QEW[Sar]EHRC(1)T;
(SEQ ID NO: 16)
[Sar]C(1)|[1-Me-Trp]QEWGEHRC(1)[Sar];
(SEQ ID NO: 17)
[Sar]C(1)IWQDWGEHRC(1)T;
(SEQ ID NO: 18)
FC(1)|[1-Me-Trp]QDW[Sar]EHRC(1)[Sar];
(SEQ ID NO: 19)
FC(1)|[1-Me-Trp]QDW[Sar]EHRC(1)T;
(SEQ ID NO: 20)
FC(1)|[1-Me-Trp]QDWGEHKC(1)[Sar];
(SEQ ID NO: 21)
FC(1)|[1-Me-Trp]QDWGEHRC(1)[Sar];
(SEQ ID NO: 22)
FC(1)|[1-Me-Trp]QDWGEHRC(1)E;
(SEQ ID NO: 23)
FC(1)|[1-Me-Trp]QDWGEHRC(1)S;
(SEQ ID NO: 24)
FC(1)|[1-Me-Trp]QDWGEHRC(1)T;
(SEQ ID NO: 25)
FC(1)|[1-Me-Trp]QEWGEHRC(1)[Sar];
(SEQ ID NO: 26)
FC(1)|[1-Nal]QDWGEHRC(1)T;
(SEQ ID NO: 27)
FC(1)|[2-Nal]QDWGEHRC(1)T;
(SEQ ID NO: 28)
FC(1)IWQDWGEHRC(1)[Sar];
(SEQ ID NO: 29)
FC(1)IWQDWGEHRC(1)T;
(SEQ ID NO: 30)
IC(1)|[1-Me-Trp]QDW[Sar]AHRC(1)[N-Me-Ile];
(SEQ ID NO: 31)
IC(1)|[1-Me-Trp]QDWGEHRC(1)[Sar];
(SEQ ID NO: 32)
IC(1)|[1-Me-Trp]QDWGEHRC(1)T;
(SEQ ID NO: 33)
IC(1)|[2-Nal]QDWGEHRC(1)[Sar];
(SEQ ID NO: 34)
IC(1)IWQDWGAHRC(1)E;
(SEQ ID NO: 35)
IC(1)IWQDWGAHRC(1)T;
(SEQ ID NO: 36)
IC(1)IWQDWGAHSC(1)T;
(SEQ ID NO: 37)
IC(1)IWQDWGDHRC(1)T;
(SEQ ID NO: 38)
IC(1)IWQDWGEHRC(1)[Sar];
(SEQ ID NO: 39)
IC(1)IWQDWGEHRC(1)E;
(SEQ ID NO: 40)
IC(1)IWQDWGEHRC(1)S;
(SEQ ID NO: 41)
IC(1)IWQDWGEHRC(1)T;
(SEQ ID NO: 42)
IC(1)IWQDWGEHSC(1)T;
(SEQ ID NO: 43)
IC(1)IWQDWGKHRC(1)T;
(SEQ ID NO: 44)
IC(1)IWQDWGRHRC(1)T;
(SEQ ID NO: 45)
IC(1)IWQDWGSHRC(1)T;
(SEQ ID NO: 46)
IC(1)IWQEWGEHRC(1)T;
(SEQ ID NO: 47)
IC(1)IWQKWGAHRC(1)T;
(SEQ ID NO: 48)
IC(1)IWQKWGEHRC(1)T;
(SEQ ID NO: 49)
YC(1)|[1-Me-Trp]QDWGEHRC(1)[Sar];
(SEQ ID NO: 50)
YC(1)|[1-Me-Trp]QDWGEHRC(1)T;
(SEQ ID NO: 51)
YC(1)|[1-Me-Trp]QEWGEHRC(1)[Sar];
(SEQ ID NO: 52)
YC(1)|[2-Nal]QDWGEHRC(1)T;
(SEQ ID NO: 53)
YC(1)IWQDWGEHRC(1)T;
(SEQ ID NO: 54)
YC(1)|[1-Me-Trp]QDWGEH[K*]C(1)[Sar];
and
(SEQ ID NO: 55)
YC(1)|[1-Me-Trp]QEW[Sar]EHRC(1)[Sar];
wherein * indicates that the amino acid residue
bears a lipophilic group ϕ covalently attached
to its side chain

In some embodiments, R1 is absent or is a sequence of 1 to 6 amino acid residues selected from A, E, G, L, K, F, P, S, T, W, R, V or Sar, or a corresponding D form thereof, and/or R2 may be a sequence of 1 to 6 amino acid residues selected from A, E, G, L, K, F, P, S, T, W, R, V or Sar, or a corresponding D form thereof.

For example, R1 is selected from ESSA (SEQ ID NO: 56), AKGE (SEQ ID NO: 57), ASSE (SEQ ID NO: 58), ASES (SEQ ID NO: 59), GSAE (SEQ ID NO: 60), ESSE (SEQ ID NO: 61), ESGA (SEQ ID NO: 62), SEG, GES, ESS, EGSA (SEQ ID NO: 63), ESE, EGE, ESA, SAE, SGA, YLEA (SEQ ID NO: 64), GSA, KEK, EKG, ES, AS, SE, SA or E, and/or R2 is selected from GAES (SEQ ID NO: 65), EYGS (SEQ ID NO: 66), EGYA (SEQ ID NO: 67), EAGS (SEQ ID NO: 68), EAKS (SEQ ID NO: 69), EKSA (SEQ ID NO: 70), EGGS (SEQ ID NO: 71), EGGA (SEQ ID NO: 72), ESSG (SEQ ID NO: 73), ESAG (SEQ ID NO: 74), GEES (SEQ ID NO: 75), AEES (SEQ ID NO: 76), ESEG (SEQ ID NO: 77), AEGS (SEQ ID NO: 78), ESGS (SEQ ID NO: 79), SEGA (SEQ ID NO: 80), SEG, ESG, EAG, GAE, EGEA (SEQ ID NO: 81), EGE, EA, E, GE, EG, EKE or EKP.

In alternative embodiments, R1 is absent or is a sequence of 1 to 6 amino acid residues selected from A, E, G, L, K, F, P, S, T, W, Y, R, V, Sar, εLys, γGlu, βAsp, or βAla, or a corresponding D form thereof, or Peg3, Peg4, or 8-aminooctanoyl, or derivatives thereof.

In some embodiments, R1 is absent or is a sequence of 1 to 6 amino acid residue selected from A, E, G, L, K, F, P, S, T, W, Y, R, V or Sar, or a corresponding D form thereof.

For example, R1 may be absent or a sequence of 1 to 6 amino acid residues selected from A, E, G, K, S and Y, or a corresponding D-form thereof.

A lipophilic group Φ may be covalently linked to the side chain of one or more of the residues in Y1, especially to the side chain of a lysine residue (which may be designated K*). It may be desirable that the residue bearing Φ is at the N-terminus of Y1.

Examples of sequences for the group R1 include:
{d}Y, EGSE (SEQ ID NO: 82), AGSE (SEQ ID NO: 83), SASE (SEQ ID NO: 84), EYSE (SEQ ID NO: 85), GSE, ASE, ESSA (SEQ ID NO: 56), KGSA (SEQ ID NO: 86), AKGE (SEQ ID NO: 57), ASGE (SEQ ID NO: 87), ASSE (SEQ ID NO: 58), ASES (SEQ ID NO: 59), GSAE (SEQ ID NO: 60), ESSE (SEQ ID NO: 61), ESGA (SEQ ID NO: 62), SEG, GES, ESS, EGSA (SEQ ID NO: 63), ESE, EGE, ESA, SAE, SGA, YLEA (SEQ ID NO: 64), GSA, KEK, EKG, ES, RS, SR, AE, TE, KE, GE, FE, YE, AS, SE, RS, SR, SA, GE, S, Y and E.

In some embodiments, R1 is two amino acid residues in length, for example, AE, TE, KE, GE, FE, YE, AS, SE, SA, or GE; preferably AE, TE, KE, GE, FE, YE, SE, or GE.

In some embodiments, R1 is one amino acids in length, for example, E.

As mentioned above, a lipophilic group Φ may be covalently linked to the side chain of one or more of the residues in Y1, especially to the side chain of a lysine residue (which may be designated K*), e.g. to yield a sequence K*GSA (SEQ ID NO: 325).

R2 may be absent or is a sequence of 1 to 8 amino acid residues selected from A, E, G, L, K, F, P, S, T, W, Y, R, V, Sar, cLys, γGlu, βAsp, or βAla, or a corresponding D form thereof; or Peg3, Peg4, or 8-aminooctanoyl, or derivatives thereof.

For example, R2 may be absent or a sequence of 1 to 8 amino acid residues selected from A, E, G, K, S, γGlu, Peg3 or 8-aminooctanoyl or selected from A, E, G, K and S.

When K is present in R2, it may be desirable that K is present at the C-terminus of R2.

A lipophilic group Φ may be covalently linked to the side chain of one or more of the residues in Y2, especially to the side chain of a lysine residue. It may be desirable that the residue bearing is at the C-terminus of Y2.

Examples of sequences for the group R2 include:
EGASGSG (SEQ ID NO: 88), EGAGSG (SEQ ID NO: 89), EGASAG (SEQ ID NO: 90), EGAGAG (SEQ ID NO: 91), EGESGSG (SEQ ID NO: 92), EGEGSG (SEQ ID NO: 93), EGESAG (SEQ ID NO: 94), EGEGAG (SEQ ID NO: 95), EK[γGlu]AK (SEQ ID NO: 96), EGEGG (SEQ ID NO: 97), EGAGG (SEQ ID NO: 98), EGESS (SEQ ID NO: 99), GAESK (SEQ ID NO: 100), EGAK (SEQ ID NO: 101), EGEK (SEQ ID NO: 102), EGG, EGK, EGKK (SEQ ID NO: 103), EGS, EK, EGA, EGAK (SEQ ID NO: 101), EK[γGlu], EK[γGlu]-K (SEQ ID NO: 104), EGE-[Peg3, EGE[Peg3]-K (SEQ ID NO: 105), EGE[Peg3][Peg3], EGE[Peg3][Peg3]-K (SEQ ID NO: 106), EGE[Peg3][Peg3][Peg3], GESESE (SEQ ID NO: 107), GAESES (SEQ ID NO: 108), EGESES (SEQ ID NO: 109), EGESESK (SEQ ID NO: 110), EGE[Peg3]-ES (SEQ ID NO: 111), EGE[Peg3]-ESK (SEQ ID NO: 112), GESESE (SEQ ID NO: 107), EGE-[8-aminooctanoyl], EGE-[8-aminooctanoyl]-K (SEQ ID NO: 113), EGE-[8-aminooctanoyl]-EK (SEQ ID NO: 114), EGEGGG (SEQ ID NO: 115), EGEGGGK (SEQ ID NO: 116), EK[γGlu]GGG (SEQ ID NO: 117), EK[]Glu]GGGK (SEQ ID NO: 118), EGE-[8-aminooctanoyl]-E (SEQ ID NO: 119), GAES (SEQ ID NO: 65), EYGS (SEQ ID NO: 66), EGYA (SEQ ID NO: 67), EAGS (SEQ ID NO: 68), EAKS (SEQ ID NO: 69), EKSA (SEQ ID NO: 70), ESGA (SEQ ID NO: 62), EGGS (SEQ ID NO: 71),

EGGA (SEQ ID NO: 72), ESSG (SEQ ID NO: 73), ESAG (SEQ ID NO: 74), GEES (SEQ ID NO: 75), AEES (SEQ ID NO: 76), ESEG (SEQ ID NO: 77), AEGS (SEQ ID NO: 78), ESGS (SEQ ID NO: 79), SEGA (SEQ ID NO: 80), SEG, EGK, , ESG, EAG, GAE, EGEA (SEQ ID NO: 81), EGE, EA, E, S, GE, GEK, EG, EA, EKE and EKP.

Examples of sequences for the group R2 include:
EGASGSG (SEQ ID NO: 88), EGAGSG (SEQ ID NO: 89), EGASAG (SEQ ID NO: 90), EGAGAG (SEQ ID NO: 91), EGESGSG (SEQ ID NO: 92), EGEGSG (SEQ ID NO: 93), EGESAG (SEQ ID NO: 94), EGEGAG (SEQ ID NO: 95), EK[γGlu]AK (SEQ ID NO: 96), EK[γGlu]A (SEQ ID NO: 266), EGEGG (SEQ ID NO: 97), EGAGG (SEQ ID NO: 98), EGESS (SEQ ID NO: 99), GAESK (SEQ ID NO: 100), EGAK (SEQ ID NO: 101), EGEK (SEQ ID NO: 102), EGG, EGK, EGKK (SEQ ID NO: 103), EGS, EK, EGA, EGAK (SEQ ID NO: 101), EK[γGlu], EK[γGlu]-K (SEQ ID NO: 104), EGE[Peg3], EGE[Peg3]-K (SEQ ID NO: 105), EGE[Peg3][Peg3], EGE[Peg3][Peg3]-K (SEQ ID NO: 106), EGE[Peg3][Peg3][Peg3], EGE[Peg3][Peg3][Peg3]-K (SEQ ID NO: 120), GESESE (SEQ ID NO: 107), GAESES (SEQ ID NO: 108), EGESES (SEQ ID NO: 109), EGESESK (SEQ ID NO: 110), EGE[Peg3]-ES (SEQ ID NO: 111), EGE[Peg3]-ESK (SEQ ID NO: 112), GESESE (SEQ ID NO: 107), EGE-[8-aminooctanoyl], EGE-[8-aminooctanoyl]-K (SEQ ID NO: 113), EGE-[8-aminooctanoyl]-EK (SEQ ID NO: 114), EGEGGG (SEQ ID NO: 115), EGEGGGK (SEQ ID NO: 116), EK[γGlu]GGG (SEQ ID NO: 117), EK[γGlu]GGGK (SEQ ID NO: 118), EGE-[8-aminooctanoyl]-E (SEQ ID NO: 119), E[Peg3][Peg3], E[Peg3][Peg3]-K, EA[Peg3][Peg3], EA[Peg3][Peg3]-K, GAES (SEQ ID NO: 65), EYGS (SEQ ID NO: 66), EGYA (SEQ ID NO: 67), EAGS (SEQ ID NO: 68), EAKS (SEQ ID NO: 69), EKSA (SEQ ID NO: 70), ESGA (SEQ ID NO: 62), EGGS (SEQ ID NO: 71), EGGA (SEQ ID NO: 72), ESSG (SEQ ID NO: 73), ESAG (SEQ ID NO: 74), GEES (SEQ ID NO: 75), AEES (SEQ ID NO: 76), ESEG (SEQ ID NO: 77), AEGS (SEQ ID NO: 78), ESGS (SEQ ID NO: 79), SEGA (SEQ ID NO: 80), SEG, EGK, ESG, EAG, GAE, EGEA (SEQ ID NO: 81), EGE, EA, E, S, GE, GEK, EG, EA, EKE and EKP.

As mentioned above, a lipophilic group Φ may be covalently linked to the side chain of one or more of the residues in Y2, especially the side chain of a lysine residue, e.g. to yield a sequence EK[γGlu]AK* (SEQ ID NO: 121), EGKK* (SEQ ID NO: 122), EK[γGlu]K* (SEQ ID NO: 123), EGE[Peg3]-K* (SEQ ID NO: 124), , EGESESK* (SEQ ID NO: 125), EGE[Peg3]-ESK* (SEQ ID NO: 126), EGE-[8-aminooctanoyl]-K* (SEQ ID NO: 127), EGE-[8-aminooctanoyl]-EK* (SEQ ID NO: 128), EGEGGGK* (SEQ ID NO: 129), EK[γGlu]GGGK* (SEQ ID NO: 130), EGE[Peg3][Peg3]-K* (SEQ ID NO: 131), GAESK* (SEQ ID NO: 132), EGAK* (SEQ ID NO: 133), EGEK* (SEQ ID NO: 134), EGK* EGE[Peg3]-ESK* (SEQ ID NO: 135), GESESEK* (SEQ ID NO: 136), GEK* or EK*.

As mentioned above, a lipophilic group Φ may be covalently linked to the side chain of one or more of the residues in Y2, especially the side chain of a lysine residue, e.g. to yield a sequence EK[γGlu]AK* (SEQ ID NO: 121), EGKK* (SEQ ID NO: 122), EK[γGlu]K* (SEQ ID NO: 123), EGE[Peg3]-K* (SEQ ID NO: 124), EGESESK* (SEQ ID NO: 125), EGE[Peg3]-ESK* (SEQ ID NO: 126), EGE-[8-aminooctanoyl]-K* (SEQ ID NO: 127), EGE-[8-aminooctanoyl]-EK* (SEQ ID NO: 128), EGEGGGK* (SEQ ID NO: 129), EK[γGlu]GGGK* (SEQ ID NO: 130), EGE[Peg3][Peg3]-K* (SEQ ID NO: 131), EGE[Peg3][Peg3][Peg3]-K* (SEQ ID NO: 137), E[Peg3][Peg3]-K*, EA[Peg3][Peg3]-K*, GAESK* (SEQ ID NO: 132), EGAK* (SEQ ID NO: 133), EGEK* (SEQ ID NO: 134), EGK* EGE[Peg3]-ESK* (SEQ ID NO: 135), GESESEK* (SEQ ID NO: 136), GEK* or EK*.

Where R1 or R2 is one amino acid in length, it may be a D amino acid, e.g. {d}Y.

R1 and R2 may independently be present or absent. It may be desirable that R2 is present. Without wishing to be bound by any particular theory, it is believed that the presence of R1 and/or R2 may improve the stability of the compounds.

Preferred classes of compstatin analogues and exemplified compounds are discussed further below.

In a further aspect, the present invention provides a composition comprising a compstatin analogue of the present invention, or a pharmaceutically acceptable salt or solvate thereof, in admixture with a carrier. In some instances, the composition is a pharmaceutical composition and the carrier is a pharmaceutically acceptable carrier.

In a further aspect, the present invention provides a pharmaceutical composition comprising a compstatin analogue of the present invention, or a pharmaceutically acceptable salt or solvate thereof, in admixture with a pharmaceutically acceptable carrier, excipient or vehicle.

In a further aspect, the present invention provides a compstatin analogue of the present invention for use in therapy.

In a further aspect, the present invention provides a compstatin analogue of the present invention for use in a method of inhibiting complement activation. By way of example, inhibiting complement activation includes one or more biological activities selected from (1) binding to C3 protein, (2) binding to C3b protein and/or (3) inhibiting the cleavage of native C3 by C3 convertases. Examples of disease or condition that may be treated using the compstatin analogues of the present invention are discussed below.

In a further aspect, the present invention provides a compstatin analogue of the present invention for use in a method of inhibiting complement activation that occurs during cell or organ transplantation.

In a further aspect, the present invention provides a method of inhibiting complement activation for treating a subject in need thereof, the method comprising administering to the subject a compstatin analogue of the present invention thereby to inhibit complement activation in the subject. Examples of disease or condition that may be treated using the compstatin analogues of the present invention are discussed below.

In a further aspect, the present invention provides an ex vivo method of inhibiting complement activation during extracorporeal shunting of a physiological fluid, the method comprising contacting the physiological fluid with a compstatin of the present invention, thereby to inhibiting complement activation.

In a further aspect, the present invention provides the use of a compstatin analogue of the present invention in the preparation of a medicament for inhibiting complement activation. Examples of disease or condition that may be treated using the compstatin analogues of the present invention are discussed below.

Embodiments of the present invention will now be described by way of example and not limitation.

### DESCRIPTION OF THE FIGURES

FIG. 1 (a-f): Normalized “ex vivo” activity of the alternative complement pathway over time after administration of a test compound at time 0 to one or two non-human primates. Compounds were given subcutaneously at a dose of 1840 nmol/kg. Complement activity (alternative pathway) was measured using the Wieslab kit. Activity was normalized using the predose (0) sample (set to 100%) and the negative control included in the kit. Normalized activity or average normalized activity for both animals and standard deviation is shown. (a) compound 61 (2 animals), (b) compound 123, compound 126 & comp 128, all with one animal per compound and Cp40 (2 animals), (c) compound 107, compound 111, compound 118 & compound 119 all with 2 animals per compound, (d) compound 104 & compound 106 with 2 animals per compound, (e) compound 54 (2 animals), and compound 122, compound 124, compound 139, and compound 140 all with 1 animal per compound, and (f) compound 141, compound 142, compound 127 and compound 130, all with one animal per compound.

### DETAILED DESCRIPTION

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Various publications, including patents, published applications, technical articles and scholarly articles are cited throughout the specification. Each of these cited publications is incorporated by reference herein, in its entirety.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well known and commonly used in the art.

Each embodiment of the invention described herein may be taken alone or in combination with one or more other embodiments of the invention.

Unless specified otherwise, the following definitions are provided for specific terms which are used in the present written description.

### Definitions

Throughout this specification, the word “comprise”, and grammatical variants thereof, such as “comprises” or “comprising”, will be understood to imply the inclusion of a stated integer or component, or group of integers or components, but not the exclusion of any other integer or component, or group of integers or components.

The singular forms “a,” “an,” and “the” include the plurals unless the context clearly dictates otherwise.

The term “including” is used to mean “including but not limited to”. “Including” and “including but not limited to” may be used interchangeably.

The terms “patient”, “subject” and “individual” may be used interchangeably. A subject may be a mammal, including a human or a non-human mammal, such as a non-human primate (e.g. ape, Old World monkey or New World monkey), livestock animal (e.g. bovine or porcine), companion animal (e.g. canine or feline) or laboratory animal such as a rodent (e.g. mouse or rat).

Throughout the present description and claims the conventional three-letter and one-letter codes for naturally occurring amino acids are used, i.e. A (Ala), G (Gly), L (Leu), I (Ile), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro); as well as generally accepted three-letter codes for other a-amino acids, such as norleucine (Nle), sarcosine (Sar), α-aminoisobutyric acid (Aib), 2,3-diaminopropanoic acid (Dap), 2,4-diaminobutanoic acid (Dab) and 2,5-diaminopentanoic acid (ornithine; Orn), 1-methyl-tryptophan(1-Me-Trp, 1Me-Trp or 1MeTrp), 1-formyl-tryptophan (1-For-Trp or 1For-Trp or 1ForTrp), 1-naphathalin (1-Nal or 1Nal), 2-naphathalin (2-Nal or 2Nal), 5-methyl-tryptophan (5-Me-Trp or 5Me-Trp or 5MeTrp), p-Benzoyl-phenylalanine (Bpa) 2-indanylglycine (2lgl or 2-Igl). Other a-amino acids may be shown in square brackets “[ ]” (e.g. “[Nle]”) when used in a general formula or sequence in the present specification, especially when the rest of the formula or sequence is shown using the single letter code. The 20 “naturally occurring” amino acids listed above are those which are encoded by the standard genetic code, and may also be referred to as “proteinogenic” amino acids.

Gamma-Glu and beta-Asp, also referred to as γGlu (γ-Glu) and βAsp (β-Asp) (or isoGlu and isoAsp), refers to glutamate or aspartate participating in peptide bonds via the γ- or β-carboxylic acid respectively (normally regarded as the side chain carboxyl groups), rather than the conventional configuration. Similarly, εLys or isoLys refers to lysine participating in a peptide bond via the epsilon amino group (normally regarded as the side chain amino group) rather than the alpha amino group.

Beta-Ala, also referred to as β-Ala or βAla, refers to 3-aminopropanoic acid.

Peg3 refers to a residue of 8-amino-3,6-dioxaoctanoic acid (also known as {2-[2-aminoethoxy]ethoxy}acetic acid) and Peg4 refers to a residue of 11-amino-3,6,9-trioxaundecanoic acid. The residue may also be denoted [8-Amino-3,6-dioxaoctanoyl].

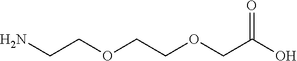 [SMILES: NCCOCCOCC(=O)O]

8-amino-3,6-dioxaoctanoic acid (Peg3)

Unless otherwise specified, amino acid residues in peptides of the invention are of the L-configuration. However, in some instances, D-configuration amino acids may be incorporated.

In the present context, an amino acid code written with a small letter represents the D-configuration of said amino acid, e.g. “k” represents the D-configuration of lysine (K), or a D-configuration amino acid may be written as (d)X or {d}X, where X is the amino acid, e.g. (d)Y or {d}Y represents the D-configuration of tyrosine (Y).

Cysteine residues shown as “C(1)” indicate that their sid-chains participate in a disulphide bond. Thus there will typically be two such residues in any given molecule.

The terminal groups present at the N- and C-termini of the peptide backbone are designated Y1 and Y2 respectively. Thus Y1 is bonded to the nitrogen atom of the N-terminal amino group and Y2 is bonded to the C-terminal carbonyl carbon atom.

Y1=hydrogen (also indicated as “H-” or “Hy-”) indicates a hydrogen atom, corresponding to the presence of a free primary or secondary amino group at the N-terminus. Y1=acetyl (“Ac”) indicates the presence of an N-terminal secondary acetyl amide group.

Y2=“OH” or “NH₂” indicates the presence of a carboxy (COOH) group or an amido (CONH₂) group at the C-terminus of the molecule.

Either or both of Y1 and Y2 may alternatively be a lipophilic group Φ. Typicaly, only one of Y1 or Y2 will be a lipophilic group Φ.

In some embodiments, whether or not the molecule comprises a lipophilic group elsewhere, Y2 is NH₂ or OH. In some embodiments, Y1 is hydrogen or acetyl, and Y2 is OH or NH₂.

In some embodiments, whether or not the molecule comprises a lipophilic group elsewhere, Y2 is NH₂. In some embodiments, Y1 is hydrogen or acetyl, and Y2 is NH₂.

In some embodiments, whether or not the molecule comprises a lipophilic group elsewhere, Y2 is NH₂ amd Y1 is acetyl.

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein. The term “about” as used herein when referring to a measurable value such as an amount, a temporal duration, and the like, is meant to encompass variations of ±20% or ±10%, in some embodiments ±5%, in some embodiments ±1%, and in some embodiments ±0.1% from the specified value, as such variations are appropriate to make and used the disclosed compounds and compositions.

The term “full length compstatin” as used herein refers to a 27 amino acid peptide having the sequence IC(1)VVQDWGHHRC(1)TAGHMANLTSHASAI (SEQ ID NO: 138), wherein C(1) denotes the cysteine residue linked by a disulphide bond. As described above, a truncated form of full length compstatin, the tridecapeptide Ile¹-Cys²-Val³-Val⁴-Gln⁵-Asp⁶-Trp⁷-Gly⁸-His⁹-His¹⁰-Arg¹¹-Cys¹²-Thr¹³-NH₂ (SEQ ID NO: 1) linked by a disulphide bond between the cysteine residues at positions 2 and 12 retains the activity of the full length peptide. An N-terminally acetylated version of this tridecapeptide peptide is referred to herein as “Ac-compstatin”.

The term “compstatin analogue” refers to a modified Ac-compstatin comprising one or more substitutions of natural and unnatural amino acids, or amino acid analogs, as well as modifications within or between various amino acids, as described in greater detail herein. A compstatin analogue may comprise about 1, 2, 3, 4 or 5 amino acid modifications relative to Ac-compstatin. A compstatin analogue may comprise 5, 6, 7, 8 or more amino acid modifications relative to Ac-compstatin. A compstatin analogue may comprise about 5, 6, 7 or 8 amino acid modifications relative to Ac-compstatin.

The term “analogue” is frequently used for a protein or peptide in question before it undergoes further chemical modification (derivatisation), and in particular acylation. The product resulting from such a chemical modification (derivatisation) is sometimes referred to as a “derivative” or “acylated analogue”. However, in the context of this application, the term “analogue” designates analogues of Ac-compstatin as well as (the acylated) derivatives of such Ac-compstatin analogues.

When referring to the position of amino acids or analogs within Ac-compstatin or compstatin analogs, the positions are numbered from 1 (Ile in compstatin) to 13 (Thr in compstatin). For example, the Gly residue occupies “position 8.” As used to describe the compstatin analogue peptides of the present invention “C(1)” denotes a disulphide bond between the respective cysteine residues in the compstatin analogue.

The terms “pharmaceutically active” and “biologically active” refer to the ability of the compounds of the invention to bind C3 or fragments thereof and inhibit complement activation. The biological activities of compstatin analogs may be measured by one or more of several art-recognized assays, as described in greater detail herein.

As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

“Hydrophobic” or “non-polar” are used synonymously herein, and refer to any inter-or intra-molecular interaction not characterized by a dipole.

As used herein, “pharmaceutically-acceptable salts” refers to derivatives of the disclosed compounds wherein the parent compound is modified by making acid or base salts thereof. Examples of pharmaceutically-acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound that has been prepared by the addition of an acid. The pharmaceutically-acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds of the present invention may exist as zwitterions. All forms of the compounds, including free acid, free base, and zwitterions, are contemplated to be within the scope of the present invention.

### Compstatin Analogues

Ac-Compstatin, an N-terminally acetylated 13 amino acid peptide, is known to bind to C3 and prevent C3 convertase-mediated cleavage. Since its discovery by phage display, modification to the 13 amino acid Ac-Compstatin sequence has been carried out in an effort to find analogues with increased biological activity. However, in the core sequence between the two cysteines residues at positions 2 and 12, alanine scanning experiments have previously produced analogues showing only modest improvements in biological activity, with few modifications being tolerated. The modifications include changing the valine at position 4 to tryptophan, or a tryptophan analogue, that leads to an increase in biological activity and changing the histidine at position 9 to alanine or analogs thereof.

In particular, previous attempts to introduce modifications to the valine residue at position 3, replacing it with glycine, alanine, D-valine or leucine have been shown to lead to a decrease in biological activity. In contrast to these prior art findings, the present inventors surprisingly found that a change of valine to isoleucine is well tolerated and provides improvements in biological activity, as shown in the Examples below.

Without wishing to be bound by any specific theory, the present inventors reasoned that this modification might be combined with introduction of one or more polar or charged amino acids in the core sequence and may be used as an approach to increase the ability of the compstatin peptides to solubilize. Initially, glutamic acid or serine at position 9 were combined with valine 3 and led to a decrease in activity compared to the reference sequence 4W9A. However, when these changes were combined with the introduction of isoleucine at position 3, a surprising increase in biological activity was observed, in particular for the combination of isoleucine at position 3 and glutamic acid at position 9. This observation correlates with improved binding to C3 as measured by surface plasmon resonance (SPR), see Table 7.

In a further series of experiments to validate these findings, compstatin peptides with glutamic acid at position 9 are combined with different substitutions in position 3 which would normally be considered “conservative” replacements for isoleucine, again showing that the peptides with isoleucine at position 3 are most active.

Taken together, these experiments show that replacing the valine residue at position 3 with isoleucine surprisingly provides compstatin peptides having increased biological activity and improved binding to C3. Furthermore, the experiments surprisingly demonstrate that these changes can be readily combined with other modifications in the core sequence of the compstatin analogues and with addition of N and C-terminal sequences, for example for improving the solubility of the compstatin peptides, e.g. at higher concentrations.

Introduction of isoleucine instead of valine at position 3 of a further prior art compound designated “Cp40” (Qu et al., Immunobiology 2013, 281(4): 496-505; also referred to in that paper as “peptide 14) also increased the binding affinity to C3 as measured by SPR.

In any embodiment X1 may be Y, I or F. In any embodiment, X4 may be W, V, 1-Nal, 2-Nal or 1-Me-Trp. In any embodiment, X6 may be E or D. In any embodiment, X9 may be A, E, D, K or S. In any embodiment, X13 may be T, S, E, I, Sar, K, or G. In any embodiment, X13 may be T, I, S, E, K or Sar. In any embodiment, X13 may be T, S, E or Sar.

### Lipophilic Substituents

The compstatin analogues may bear a lipophilic group, designated Φ.

The lipophilic group may be covalently linked to the N-terminus and/or the C terminus of the molecule, i.e. Y1 may be Φ (in place of H or Ac) and/or Y2 may be Φ (in place of OH or NH2).

Additionally or alternatively, the lipophilic group may be covalently linked to the side chain of an amino acid residue within the analogue. The residue may be part of R1, R2 or the compstatin analogue portion X1-X13 of the molecule.

The lipophilic group Φ is typically attached via an acyl group. The modification may therefore be termed acylation but can also be refered to as lipidation.

The lipophilic group includes a long chain alkylene group derived from a fatty acid, termed Z¹ herein and referred to as the lipophilic substituent. Without wishing to be bound by theory, it is believed that a lipophilic substituent binds plasma proteins (e.g. albumin) in the blood stream, thus shielding the compounds employed in the context of the invention from enzymatic degradation, and thereby enhancing the half-life of the compounds. The lipophilic substituent may also modulate the potency of the compound.

Z¹ may be attached directly to the amino acid sequence (including the R1 and R2 extensions, or as Y1) or via a spacer Z² as defined herein.

In other words, Φ may be Z¹— or Z¹—Z²—.

Where Y1 is Φ, Φ is preferably Z¹—.

Where the lipophilic group Φ is linked to an amino acid side chain (i.e. where Y1 is hydrogen or Ac) Φ may preferably be Z¹—Z²—.

In certain embodiments, only one amino acid side chain is conjugated to a lipophilic substituent. In other embodiments, two amino acid side chains are each conjugated to a lipophilic substituent. In yet further embodiments, three or even more amino acid side chains are each conjugated to a lipophilic substituent. When a compound contains two or more lipophilic substituents, they may be the same or different substituents.

In certain embodiments, only one lipophilic group Φ is present in the molecule.

The term “conjugated” is used here to describe the covalent attachment of one identifiable chemical moiety to another, and the structural relationship between such moieties. It should not be taken to imply any particular method of synthesis. The one or more spacers Z², when present, are used to provide a spacing between the compound and the lipophilic substituent Z¹.

A lipophilic substituent may be attached to an N-terminal nitrogen, or to an amino acid side chain or to a spacer via an ester, a sulphonyl ester, a thioester, an amide or a sulphonamide. Accordingly, it will be understood that a lipophilic substituent may include an acyl group, a sulphonyl group, an N atom, an O atom or an S atom which forms part of the ester, sulphonyl ester, thioester, amide or sulphonamide.

Suitably, an acyl group in the lipophilic substituent forms part of an amide or ester with the N-terminal nitrogen, or amino acid side chain, or the spacer. The lipophilic substituent may include a hydrocarbon chain having 10 to 24 carbon (C) atoms, e.g. 10 to 22 C atoms, e.g. 10 to 20 C atoms. Preferably, it has at least 11 C atoms, and preferably it has 18 C atoms or fewer. For example, the hydrocarbon chain may contain 12, 13, 14, 15, 16, 17 or 18 carbon atoms. The hydrocarbon chain may be linear or branched and may be saturated or unsaturated.

The hydrocarbon chain may incorporate a phenylene or piperazinylene moiety in its length as, for example, shown below (wherein — represents the points of attachment within the chain). These groups should be “counted” as 4 carbon atoms in the chain length.

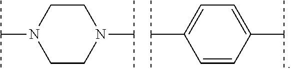

From the discussion above, it will be understood that the hydrocarbon chain may be substituted with a moiety which forms part of the attachment to the amino acid side chain or the spacer, for example an acyl group, a sulphonyl group, an N atom, an O atom or an S atom. Most preferably, the hydrocarbon chain is substituted with an acyl group, and accordingly the hydrocarbon chain may be part of an alkanoyl group, for example a dodecanoyl, 2-butyloctanoyl, tetradecanoyl, hexadecanoyl, heptadecanoyl, octadecanoyl or eicosanoyl group. Alternatively, Z¹ groups are derived from long-chain saturated α,ω-dicarboxylic acids of formula HOOC—(CH₂)₁₂₋₂₂—COOH, preferably from long-chain saturated α,ω-dicarboxylic acids having an even number of carbon atoms in the aliphatic chain.

In other words, Z¹ may be A—C₁₂₋₂₂alkylene—(CO)—, where A is H or —COOH, and wherein the akylene may be linear or branched and may be saturated or unsaturated, and may optionally incorporate a phenylene or piperazinylene moiety in its length.

For example, Z¹ may be:
Dodecanoyl i.e. H—(CH₂)₁₁—(CO)—;
Tetradecanoyl i.e. H—(CH₂)₁₃—(CO)—;
Hexadecanoyl, i.e. H—(CH₂)₁₅—(CO)—;
13-carboxytridecanoyl, i.e. HOOC—(CH₂)₁₂—(CO)—;
15-carboxypentadecanoyl, i.e. HOOC—(CH₂)₁₄—(CO)—;
17-carboxyheptadecanoyl, i.e. HOOC—(CH₂)₁₆—(CO)—;
19-carboxynonadecanoyl, i.e. HOOC—(CH₂)₁₈—(CO)—; or
21-carboxyheneicosanoyl, i.e. HOOC—(CH₂)₂₀—(CO)—

The carboxylic acid, if present, may be replaced by a bioisotere, phosphate or sulfonate. Suitable bioisoteres for carboxylic acids are known in the art and include tetrazole, acylsulfomides, acylhydroxylamine, and squaric acid derivatives.

As mentioned above, the lipophilic substituent Z¹ may be conjugated to the amino acid side chain or N-terminal nitrogen by one or more spacers Z².

When present, the spacer is attached to the lipophilic substituent and to the amino acid side chain or N-terminal nitrogen. The spacer may be attached to the lipophilic substituent and to the amino acid side chain independently by an ester, a sulphonyl ester, a thioester, an amide or a sulphonamide. Accordingly, it may include two moieties independently selected from acyl, sulphonyl, an N atom, an O atom or an S atom. The spacer may consist of a linear C₁₋₁₀hydrocarbon chain or more preferably a linear C₁₋₅ hydrocarbon chain. Furthermore the spacer can be substituted with one or more substituents selected from C₁₋₆ alkyl, C₁₋₆ alkyl amine, C₁₋₆ alkyl hydroxy and C₁₋₆ alkyl carboxy.

The spacer may be, for example, a residue of any naturally occurring or unnatural amino acid. For example, the spacer may be a residue of Gly, Pro, Ala, Val, Leu, Ile, Met, Cys, Phe, Tyr, Trp, His, Lys, Arg, Gln, Asn, Glu, Asp, γ-Glu, β-Asp, ε-Lys, Asp, Ser, Thr, Dapa, Gaba, Aib, β-Ala (i.e., 3-aminopropanoyl), 4-aminobutanoyl, 5-aminopentanoyl, 6-aminohexanoyl, 7-aminoheptanoyl, 8-aminooctanoyl, 9-aminononanoyl, 10-aminodecanoyl, 8-amino-3,6-dioxaoctanoyl. In certain embodiments, the spacer is a residue of Glu, γ-Glu, ε-Lys, β-Ala (i.e., 3-aminopropanoyl), 4-aminobutanoyl, 8-aminooctanoyl or 8-amino-3,6-dioxaoctanoyl (Peg3), 11-amino-3,6,9-trioxaundecanoic acid (Peg4) or (piperazine-1-yl)-carboxylic acid. In the present invention, γGlu and isoGlu are used interchangeably.

Z² is suitably a sequence of 1 to 6 residues of compounds selected from γGlu, βAsp, D, E, K, Orn, S, T, A, βAla, G, P, V, L, I, Y, Q, N, Dapa, Gaba, or Aib, or a corresponding D form thereof, 5-aminopentanoyl, 6-aminohexanoyl, 7-aminoheptanoyl, 8-aminooctanoyl, 9-aminononanoyl, and 10-aminodecanoyl. 8-amino-3,6-dioxaoctanoic acid (Peg3), 11-amino-3,6,9-trioxaundecanoic acid (Peg4) or (piperazine-1-yl)-carboxylic acid.

For example, Z² may be, or may comprise:

[yGlu];
[yGlu][Peg3][Peg3]-;
[(Piperazine-1-yl)-acetyl][Peg3][Peg3];
[yGlu]-G-[yGlu];
[yGlu]-K-[yGlu];
(SEQ ID NO: 139)
[YGlu]-KG-[yGlu];
or
[yGlu]-G-[Peg3][yGlu][Peg3].

Z² is suitably bound at each side by amide linkage. Other suitable linkages may be used, with the commensurate atom replacement; for example sulfinamide, sulfonamide, or ester linkages or amino, ether, or thioether linkages are envisaged.

In other words, in some aspects the lipophilic group Φ is Z¹—or Z¹—Z²—; wherein
Z¹ is A—C₁₂₋₂₂alkylene—(CO)—;
where A is H or —COOH, and wherein the akylene may be linear or branched and may be saturated or unsaturated, and may optionally incorporate a phenylene or piperazinylene moiety in its length; and
Z² is a sequence of 1 to 6 of residues of compounds selected from γ-Glu, βAsp, D, E, K, Orn, S, T, A, β-Ala, G, P, V, L, I, Y, Q, N, Dapa, Gaba, or Aib, or a correspdoning D form thereof, 5-aminopentanoyl, 6-aminohexanoyl, 7-aminoheptanoyl, 8-aminooctanoyl, 9-aminononanoyl, and 10-aminodecanoyl. 8-amino-3,6-dioxaoctanoic acid (Peg3), 11-amino-3,6,9-trioxaundecanoic acid (Peg4) or (piperazine-1-yl)-carboxylic acid, e.g. a linker selected from

[Glu],
[yGlu][Peg3][Peg3] -;
[(Piperazine-1-yl)-acetyl][Peg3][Peg3];
[YGlu]-G-[yGlu];
[YGlu)-K-[yGlu];
(SEQ ID NO: 139)
[YGlu]-KG-[yGlu];
and
[YGlu]-G-[Peg3][yGlu][Peg3].

The amino acid side chain to which the lipophilic substituent is conjugated typically includes a carboxy, hydroxyl, thiol, amide or amine group, for forming an ester, a sulphonyl ester, a thioester, an amide, or a sulphonamide with the spacer or lipophilic substituent. An amide linkage may be particularly preferred, and thus the amino acid may be any amino acid having an amine group in its side chain, although it will be clear that side chains having other functional groups are contemplated. Thus, the amino acid side chain may be a side chain of a Glu, Lys, Ser, Cys, Dbu, Dpr or Orn residue. For example, it may be a side chain of a Lys, Glu or Cys residue. Where two or more side chains carry a lipophilic substituent, they may be independently selected from those residues.

Typically, the amino acid side chain is a side chain of a Lys residue.

An example of a lipophilic substituent comprising a lipophilic moiety Z¹ and spacer Z² is shown in the formula below:

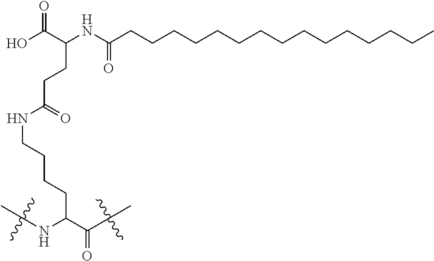 [SMILES: *NC(CCCCNC(=O)CCC(NC(=O)CCCCCCCCCCCCCCC)C(=O)O)C(*)=O]

Here, the side chain of a Lys residue is covalently attached to a γGlu spacer (Z²) via an amide linkage. A hexadecanoyl group (Z¹) is covalently attached to the γGlu spacer via an amide linkage. This combination of lipophilic moiety and spacer, conjugated to a Lys residue, may be referred to by the short-hand notation K(Hexadecanoyl-γGlu), e.g., when shown in formulae of specific compounds. γGlu can also be referred to as isoGlu, and a hexadecanoyl group as a palmitoyl group. Thus it will be apparent that the notation (Hexadecanoyl-γGlu) is equivalent to the notations (isoGlu(Palm)) or (isoGlu(Palmitoyl)) as used for example in PCT/GB2008/004121.

Alternative Z¹ groups are derived from long-chain saturated α,ω-dicarboxylic acids of formula HOOC—(CH₂)₁₂₋₂₂—COOH as exemplified below

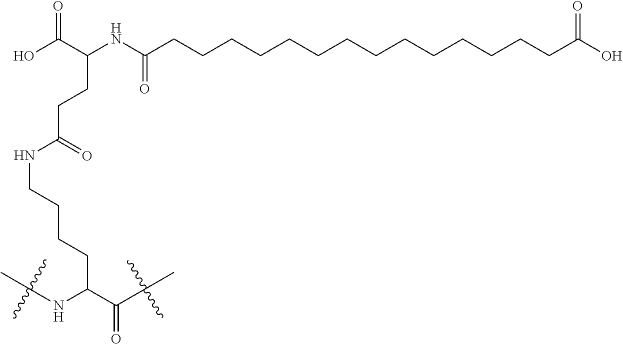 [SMILES: *NC(CCCCNC(=O)CCC(NC(=O)CCCCCCCCCCCCCCC(=O)O)C(=O)O)C(*)=O]

Here, the side chain of a Lys residue is covalently attached to a γGlu spacer (Z²) via an amide linkage. A 15-carboxypentadecanoyl group (Z¹) is covalently attached to the γGlu spacer via an amide linkage. This combination of lipophilic moiety and spacer, conjugated to a Lys residue, may be referred to by the short-hand notation K(15-carboxypentadecanoyl-γ-Glu), e.g., when shown in formulae of specific compounds. γGlu can also be referred to as isoGlu.

Certain preferred Φ groups (Z¹— and Z¹—Z²—) include:
[15-Carboxy-pentadecanoyl];
[15-carboxy-pentadecanoyl][γGlu],
[15-carboxy-pentadecanoyl][γGlu][Peg3][Peg3];
[19-carboxy-nonadecanoyl][γGlu][Peg3][Peg3];
[15-carboxy-pentadecanoyl][(Piperazine-1-yl)-acetyl][Peg3][Peg3];
[17-carboxy-heptadecanoyl][γGlu]G[γGlu];
[17-carboxy-heptadecanoyl][γGlu]K[γGlu];
[17-carboxy-heptadecanoyl][γGlu]KG[γGlu] (SEQ ID NO: 267);
[17-carboxy-heptadecanoyl][γGlu]G[Peg3][γGlu][Peg3];
[15-carboxy-pentadecanoyl][γGlu]G[γGlu];
[17-carboxy-heptadecanoyl];
[17-carboxy-heptadecanoyl][γGlu]
[19-carboxy-nonadecanoyl][γGlu]G[γGlu]; and
[17-carboxy-heptadecanoyl][γGlu][Peg3][Peg3].

Illustrative structures of Φ groups (Z¹— and Z¹—Z²—) are shown below, where the wavy line indicates the linkage to the peptide (to an amino acid side chain, N-terminal nitrogen, or C-terminal carbon):

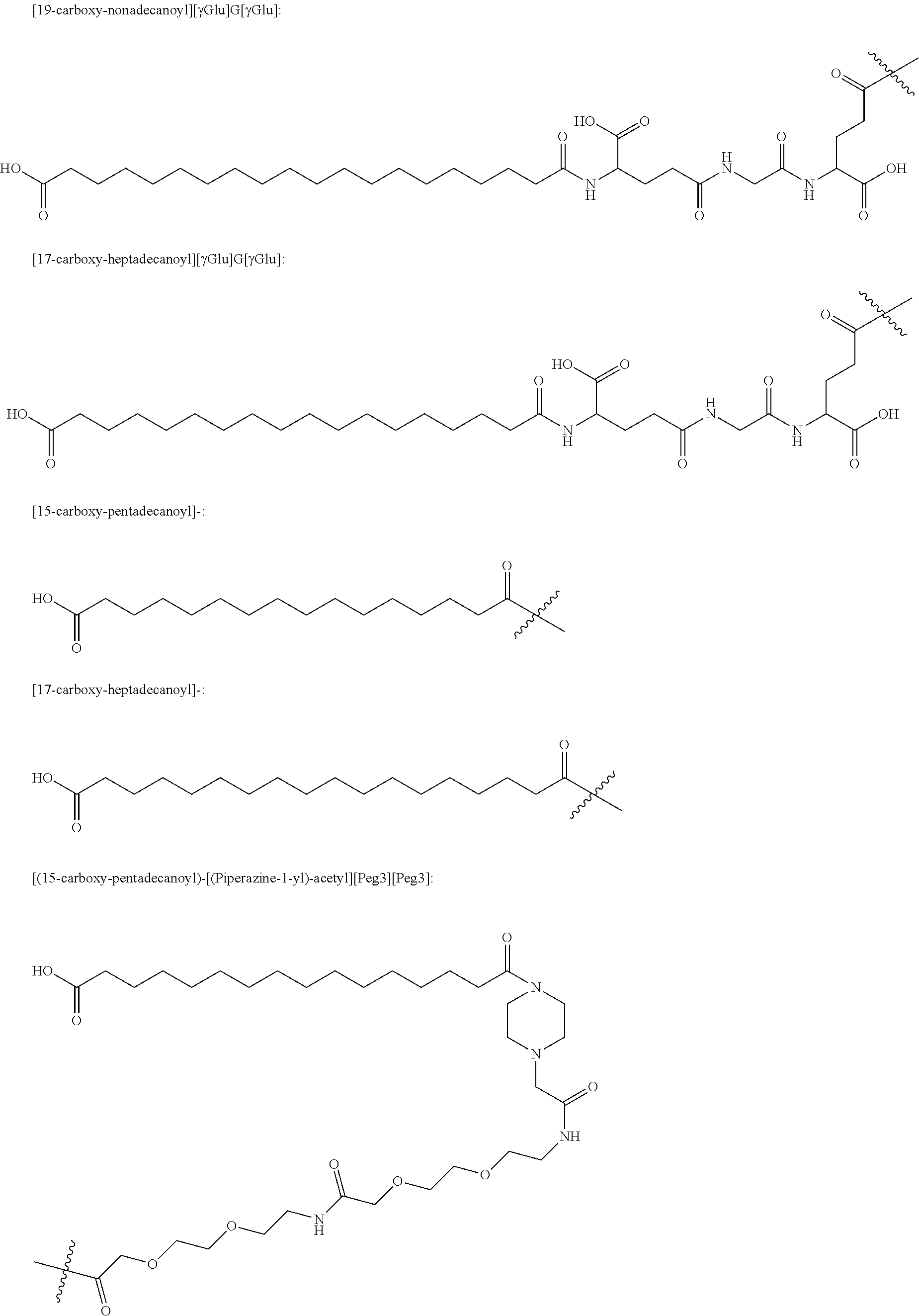 [SMILES: *C(=O)CCC(NC(=O)CNC(=O)CCC(NC(=O)CCCCCCCCCCCCCCCCCCC(=O)O)C(=O)O)C(=O)O ; *C(=O)CCC(NC(=O)CNC(=O)CCC(NC(=O)CCCCCCCCCCCCCCCCC(=O)O)C(=O)O)C(=O)O ; *C(=O)CCCCCCCCCCCCCCC(=O)O ; *C(=O)CCCCCCCCCCCCCCCCC(=O)O ; *C(=O)COCCOCCNC(=O)COCCOCCNC(=O)CN1CCN(C(=O)CCCCCCCCCCCCCCC(=O)O)CC1]

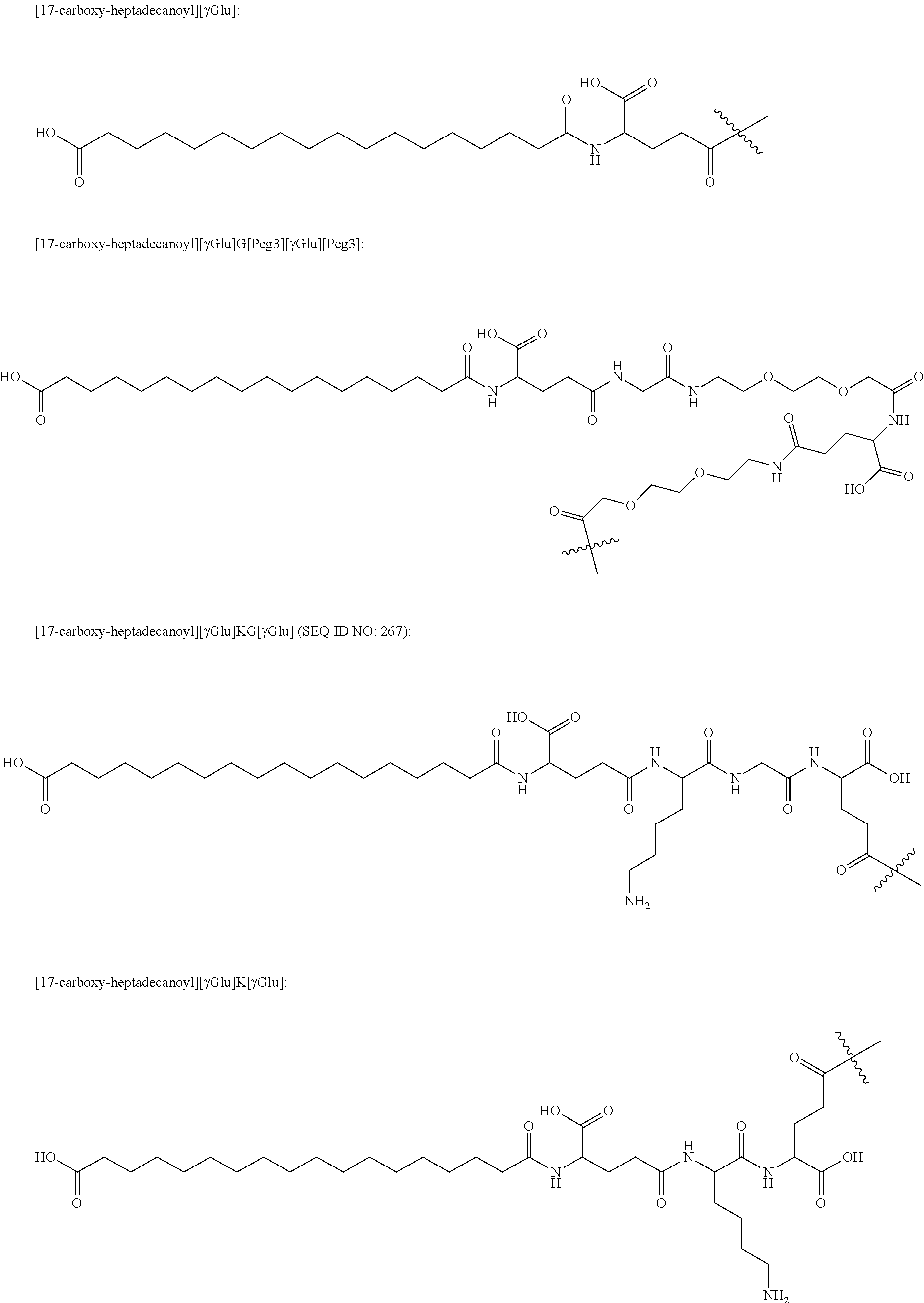 [SMILES: *C(=O)CCC(NC(=O)CCCCCCCCCCCCCCCCC(=O)O)C(=O)O ; *C(=O)COCCOCCNC(=O)CCC(NC(=O)COCCOCCNC(=O)CNC(=O)CCC(NC(=O)CCCCCCCCCCCCCCCCC(=O)O)C(=O)O)C(=O)O ; *C(=O)CCC(NC(=O)CNC(=O)C(CCCCN)NC(=O)CCC(NC(=O)CCCCCCCCCCCCCCCCC(=O)O)C(=O)O)C(=O)O ; *C(=O)CCC(NC(=O)C(CCCCN)NC(=O)CCC(NC(=O)CCCCCCCCCCCCCCCCC(=O)O)C(=O)O)C(=O)O]

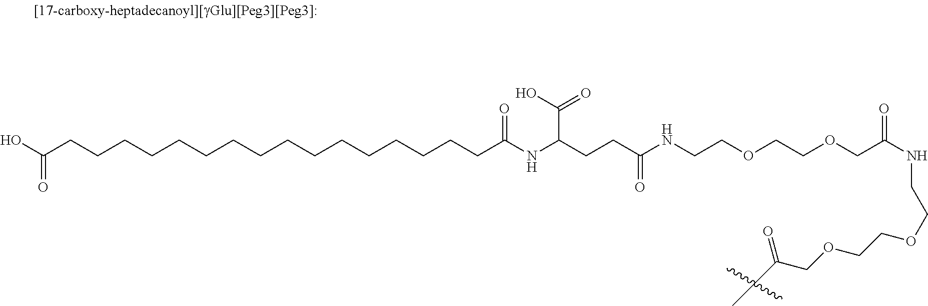 [SMILES: *C(=O)COCCOCCNC(=O)COCCOCCNC(=O)CCC(NC(=O)CCCCCCCCCCCCCCCCC(=O)O)C(=O)O]

The skilled person will be well aware of suitable techniques for preparing the compounds employed in the context of the invention. For examples of suitable chemistry, see WO98/08871, WO00/55184, WO00/55119, Madsen et al., J. Med. Chem. 50:6126-32 (2007), and Knudsen et al., J. Med Chem. 43:1664-1669 (2000), incorporated herein by reference.

In some embodiments, the compstatin analogue has a lipophilic group Φ as described above conjugated to an amino acid at one or more of positions corresponding to positions 1, 3, 4, 5, 6, 7, 8, 9, 10, 11 or 13 of the compstatin-like sequence, i.e. positions X1-X13.

In certain embodiments, the compstatin analogue has a lipophilic substituent as described above conjugated to an amino acid at one or more of positions corresponding to positions X1, X11 or X13, or to an amino acid within R1 or R2, or at the N-terminus as group Y1.

For C-terminal acylation or lipidation of peptides, well-established conjugation strategies have been developed. For example, such conjugation could be performed by click chemistry (i.e. the biorthogonal azide-alkyne conjugation reaction catalyzed by Cu(I)) or by other conjugation strategies known to the person skilled in the art of peptide chemistry.

The compstatin analogue may comprise one of the following sequences:

(SEQ ID NO: 35)
IC(1)IWQDWGAHRC(1)T
(SEQ ID NO: 41)
IC(1)IWQDWGEHRC(1)T
(SEQ ID NO: 140)
ESSAIC(1)IWQDWGEHRC(1)T
(SEQ ID NO: 141)
IC(1)|[1MeTrp]QDWGEHRC(1)T
(SEQ ID NO: 43)
IC(1)IWQDWGKHRC(1)T
(SEQ ID NO: 45)
IC(1)IWQDWGSHRC(1)T
(SEQ ID NO: 48)
IC(1)IWQKWGEHRC(1)T
(SEQ ID NO: 142)
IC(1)IWQKWGAHRC(1)TGAES
(SEQ ID NO: 53)
YC(1)IWQDWGEHRC(1)T
(SEQ ID NO: 143)
ESSAYC(1)IWQDWGEHRC(1)T
(SEQ ID NO: 17)
[Sar]C(1)IWQDWGEHRC(1)T
(SEQ ID NO: 34)
IC(1)IWQDWGAHRC(1)E
(SEQ ID NO: 38)
IC(1)IWQDWGEHRC(1)[Sar]
(SEQ ID NO: 144)
ESSAIC(1)IWQDWGEHRC(1)TGAES
(SEQ ID NO: 145)
IC(1)IWQDWGEHRC(1)TGAES
(SEQ ID NO: 46)
IC(1)IWQEWGEHRC(1)T
(SEQ ID NO: 37)
IC(1)IWQDWGDHRC(1)T
(SEQ ID NO: 44)
IC(1)IWQDWGRHRC(1)T
(SEQ ID NO: 36)
IC(1)IWQDWGAHSC(1)T
(SEQ ID NO: 42)
IC(1)IWQDWGEHSC(1)T
(SEQ ID NO: 40)
IC(1)IWQDWGEHRC(1)S
(SEQ ID NO: 39)
IC(1)IWQDWGEHRC(1)E
(SEQ ID NO: 29)
FC(1)IWQDWGEHRC(1)T
(SEQ ID NO: 146)
IC(1)IWQDWGEHRC(1)TEGE
(SEQ ID NO: 147)
IC(1)IWQDWGEHRC(1)TEA
(SEQ ID NO: 148)
IC(1)IWQDWGEHRC(1)TE
(SEQ ID NO: 149)
IC(1)IWQDWGEHRC(1)EGE
(SEQ ID NO: 150)
EGSAIC(1)IWQDWGEHRC(1)[Sar]E
(SEQ ID NO: 151)
EGSAIC(1)IWQDWGEHRC(1)T
(SEQ ID NO: 152)
EGEIC(1)IWQDWGEHRC(1)T
(SEQ ID NO: 153)
ESEIC(1)IWQDWGEHRC(1)T
(SEQ ID NO: 154)
SEIC(1)IWQDWGEHRC(1)TEA
(SEQ ID NO: 155)
EIC(1)IWQDWGEHRC(1)TE
(SEQ ID NO: 156)
EIC(1)IWQDWGEHRC(1)TEGE
(SEQ ID NO: 157)
EGEIC(1)IWQDWGEHRC(1)EGE
(SEQ ID NO: 158)
ESEIC(1)IWQDWGEHRC(1)EGE
(SEQ ID NO: 159)
KEKIC(1)IWQDWGEHRC(1)TEKE
(SEQ ID NO: 160)
EKGIC(1)IWQDWGEHRC(1)TEKP
(SEQ ID NO: 161)
IC(1)IWQDWGEHRC(1)TEGK
(SEQ ID NO: 162)
GSAIC(1)IWQDWGEHRC(1)[Sar]E
(SEQ ID NO: 163)
SAIC(1)IWQDWGEHRC(1)[Sar]E
(SEQ ID NO: 164)
SAIC(1)IWQDWGEHRC(1)TEG
(SEQ ID NO: 165)
FC(1)IWQDWGEHRC(1)TGAE
(SEQ ID NO: 166)
EGSAIC(1)IWQDWGEHRC(1)[Sar]EGE
(SEQ ID NO: 167)
EGSAFC(1)IWQDWGEHRC(1)[Sar]E
(SEQ ID NO: 168)
ESSAIC(1)IWQDWGAHRC(1)T
(SEQ ID NO: 169)
IC(1)IWQDWGAHRC(1)TGAES
(SEQ ID NO: 170)
{d}YIC(1)|[1-Me-Trp]QDW[Sar]AHRC(1)-[N-Me-Ile]
(SEQ ID NO: 171)
EGSAIC(1)|[1-Me-Trp]QDWGEHRC(1)[Sar]E
(SEQ ID NO: 172)
EGSAIC(1)|[2-Nal]QDWGEHRC(1)[Sar]E
(SEQ ID NO: 173)
IC(1)|[1-Me-Trp]QDWGEHRC(1)TGAES
(SEQ ID NO: 174)
IC(1)|[2-Nal]QDWGEHRC(1)TGAES
(SEQ ID NO: 175)
EGSAFC(1)|[1-Me-Trp]QDWGEHRC(1)[Sar]E
(SEQ ID NO: 176)
EGSAYC(1)|[1-Me-Trp]QDWGEHRC(1)[Sar]E
(SEQ ID NO: 177)
EGSAIC(1)IWQDWGEHRC(1)TE
(SEQ ID NO: 178)
EGSAFC(1)|[1-Nal]QDWGEHRC(1)TE
(SEQ ID NO: 179)
EGSAFC(1)|[1-Me-Trp]QDWGEHRC(1)TE
(SEQ ID NO: 180)
EGSAFC(1)|[1-Me-Trp]QDWGEHRC(1)EGE
(SEQ ID NO: 181)
EGSAYC(1)|[1-Me-Trp]QDWGEHRC(1)TE
(SEQ ID NO: 182)
EGSAFC(1)|[2-Nal]QDWGEHRC(1)TE
(SEQ ID NO: 183)
FC(1)|[1-Me-Trp]QDWGEHRC(1)TGAES
(SEQ ID NO: 184)
YC(1)|[1-Me-Trp]QDWGEHRC(1)TGAES
(SEQ ID NO: 185)
FC(1)|[1-Nal]QDWGEHRC(1)TGAES
(SEQ ID NO: 186)
FC(1)|[2-Nal]QDWGEHRC(1)TGAES
(SEQ ID NO: 187)
YC(1)|[2-Nal]QDWGEHRC(1)TGAES
(SEQ ID NO: 188)
YC(1)IWQDWGEHRC(1)TGAES
(SEQ ID NO: 189)
SEFC(1)|[1-Me-Trp]QDWGEHRC(1)TGAES
(SEQ ID NO: 190)
YC(1)|[1-Me-Trp]QDWGEHRC(1)TEAGS
(SEQ ID NO: 191)
YC(1)|[1-Me-Trp]QDWGEHRC(1)TESGA
(SEQ ID NO: 192)
EGSAYC(1)[1-Me-Trp]QEWGEHRC(1)[Sar]E
(SEQ ID NO: 193)
SEYC(1)|[1-Me-Trp]QDWGEHRC(1)[Sar]EA
(SEQ ID NO: 194)
FC(1)|[1-Me-Trp]QDW[Sar]EHRC(1)TGAES
(SEQ ID NO: 195)
{d}YFC(1)|[1-Me-Trp]QDW[Sar]EHRC(1)TGAES
(SEQ ID NO: 196)
SEFC(1)[1-Me-Trp]QDWGEHRC(1)[Sar]GAES
(SEQ ID NO: 197)
SEFC(1)|[1-Me-Trp]QDWGEHRC(1)[Sar]EA
(SEQ ID NO: 198)
SEFC(1)|[1-Me-Trp]QDW[Sar]EHRC(1)[Sar]EA
(SEQ ID NO: 199)
SEFC(1)|[1-Me-Trp]QDW[Sar]EHRC(1)TEA
(SEQ ID NO: 200)
SEFC(1)|[1-Me-Trp]QDWGEHRC(1)[Sar]E
(SEQ ID NO: 201)
SEFC(1)|[1-Me-Trp]QDW[Sar]EHRC(1)[Sar]E
(SEQ ID NO: 202)
EFC(1)|[1-Me-Trp]QDWGEHRC(1)[Sar]EA
(SEQ ID NO: 203)
SE[Sar]C(1)|[1-Me-Trp]QDWGEHRC(1)[Sar]EA
(SEQ ID NO: 204)
SE[Sar]C(1)|[1-Me-Trp]QDWGEHRC(1)TEA
(SEQ ID NO: 205)
SEFC(1)|[1-Me-Trp]QEWGEHRC(1)[Sar]EA
(SEQ ID NO: 206)
SEFC(1)|[1-Me-Trp]QDWGEHRC(1)SEA
(SEQ ID NO: 207)
EFC(1)|[1-Me-Trp]QDWGEHRC(1)ES
(SEQ ID NO: 208)
SEFC(1)|[1-Me-Trp]QDWGEHKC(1)[Sar]EA
(SEQ ID NO: 209)
GEFC(1)|[1-Me-Trp]QDWGEHRC(1)[Sar]EA
(SEQ ID NO: 210)
GE[Sar]C(1)|[1-Me-Trp]QDWGEHRC(1)TEA
(SEQ ID NO: 211)
SE[Sar]C(1)|[1-Me-Trp]QEW[Sar]EHRC(1)TEA
(SEQ ID NO: 212)
SE[Sar]C(1)|[1-Me-Trp]QEWGEHRC(1)[Sar]EA
(SEQ ID NO: 213)
{d}Y[Sar]C(1)|[1-Me-Trp]QDWGEHRC(1)TEA

For example, the compstatin analogue may be:

(Compound 1)
(SEQ ID NO: 35)
Ac-IC(1)IWQDWGAHRC(1)T-NH2
(Compound 2)
(SEQ ID NO: 41)
Ac-IC(1)IWQDWGEHRC(1)T-NH2
(Compound 3)
(SEQ ID NO: 140)
Ac-ESSAIC(1)IWQDWGEHRC(1)T-NH2
(Compound 4)
(SEQ ID NO: 32)
Ac-IC(1)|[1-Me-Trp]QDWGEHRC(1)T-NH2
(Compound 5)
(SEQ ID NO: 43)
Ac-IC(1)IWQDWGKHRC(1)T-NH2
(Compound 6)
(SEQ ID NO: 45)
Ac-IC(1)IWQDWGSHRC(1)T-NH2
(Compound 7)
(SEQ ID NO: 48)
Ac-IC(1)IWQKWGEHRC(1)T-NH2
(Compound 8)
(SEQ ID NO: 142)
Ac-IC(1)IWQKWGAHRC(1)TGAES-NH2
(Compound 9)
(SEQ ID NO: 53)
Ac-YC(1)IWQDWGEHRC(1)T-NH2
(Compound 10)
(SEQ ID NO: 143)
Ac-ESSAYC(1)IWQDWGEHRC(1)T-NH2
(Compound 11)
(SEQ ID NO: 17)
Ac-[Sar]C(1)IWQDWGEHRC(1)T-NH2
(Compound 12)
(SEQ ID NO: 34)
Ac-IC(1)IWQDWGAHRC(1)E-NH2
(Compound 13)
(SEQ ID NO: 38)
Ac-IC(1)IWQDWGEHRC(1)[Sar]-NH2
(Compound 14)
(SEQ ID NO: 144)
Ac-ESSAIC(1)IWQDWGEHRC(1)TGAES-NH2
(Compound 15)
(SEQ ID NO: 145)
Ac-IC(1)IWQDWGEHRC(1)TGAES-NH2
(Compound 16)
(SEQ ID NO: 46)
Ac-IC(1)IWQEWGEHRC(1)T-NH2
(Compound 17)
(SEQ ID NO: 37)
Ac-IC(1)IWQDWGDHRC(1)T-NH2
(Compound 18)
(SEQ ID NO: 44)
Ac-IC(1)IWQDWGRHRC(1)T-NH2
(Compound 19)
(SEQ ID NO: 36)
Ac-IC(1)IWQDWGAHSC(1)T-NH2
(Compound 20)
(SEQ ID NO: 42)
Ac-IC(1)IWQDWGEHSC(1)T-NH2
(Compound 21)
(SEQ ID NO: 40)
Ac-IC(1)IWQDWGEHRC(1)S-NH2
(Compound 22)
(SEQ ID NO: 39)
Ac-IC(1)IWQDWGEHRC(1)E-NH2
(Compound 23)
(SEQ ID NO: 29)
Ac-FC(1)IWQDWGEHRC(1)T-NH2
(Compound 24)
(SEQ ID NO: 146)
Ac-IC(1)IWQDWGEHRC(1)TEGE-NH2
(Compound 25)
(SEQ ID NO: 147)
Ac-IC(1)IWQDWGEHRC(1)TEA-NH2
(Compound 26)
(SEQ ID NO: 148)
Ac-IC(1)IWQDWGEHRC(1)TE-NH2
(Compound 27)
(SEQ ID NO: 149)
Ac-IC(1)IWQDWGEHRC(1)EGE-NH2
(Compound 28)
(SEQ ID NO: 150)
Ac-EGSAIC(1)IWQDWGEHRC(1)[Sar]E-NH2
(Compound 29)
(SEQ ID NO: 151)
Ac-EGSAIC(1)IWQDWGEHRC(1)T-NH2
(Compound 30)
(SEQ ID NO: 152)
Ac-EGEIC(1)IWQDWGEHRC(1)T-NH2
(Compound 31)
(SEQ ID NO: 153)
Ac-ESEIC(1)IWQDWGEHRC(1)T-NH2
(Compound 32)
(SEQ ID NO: 154)
Ac-SEIC(1)IWQDWGEHRC(1)TEA-NH2
(Compound 33)
(SEQ ID NO: 155)
Ac-EIC(1)IWQDWGEHRC(1)TE-NH2
(Compound 34)
(SEQ ID NO: 156)
Ac-EIC(1)IWQDWGEHRC(1)TEGE-NH2
(Compound 35)
(SEQ ID NO: 157)
Ac-EGEIC(1)IWQDWGEHRC(1)EGE-NH2
(Compound 36)
(SEQ ID NO: 158)
Ac-ESEIC(1)IWQDWGEHRC(1)EGE-NH2
(Compound 37)
(SEQ ID NO: 159)
Ac-KEKIC(1)IWQDWGEHRC(1)TEKE-NH2
(Compound 38)
(SEQ ID NO: 160)
Ac-EKGIC(1)IWQDWGEHRC(1)TEKP-NH2
(Compound 39)
(SEQ ID NO: 161)
Ac-IC(1)IWQDWGEHRC(1)TEGK-NH2
(Compound 40)
(SEQ ID NO: 162)
Ac-GSAIC(1)IWQDWGEHRC(1)[Sar]E-NH2
(Compound 41)
(SEQ ID NO: 163)
Ac-SAIC(1)IWQDWGEHRC(1)[Sar]E-NH2
(Compound 42)
(SEQ ID NO: 164)
Ac-SAIC(1)IWQDWGEHRC(1)TEG-NH2
(Compound 43)
(SEQ ID NO: 165)
Ac-FC(1)IWQDWGEHRC(1)TGAE-NH2
(Compound 44)
(SEQ ID NO: 166)
Ac-EGSAIC(1)IWQDWGEHRC(1)[Sar]EGE-NH2
(Compound 45)
(SEQ ID NO: 167)
Ac-EGSAFC(1)IWQDWGEHRC(1)[Sar]E-NH2
(Compound 46)
(SEQ ID NO: 168)
Ac-ESSAIC(1)IWQDWGAHRC(1)T-NH2
(Compound 47)
(SEQ ID NO: 169)
Ac-IC(1)IWQDWGAHRC(1)TGAES-NH2
(Compound 48)
(SEQ ID NO: 170)
H-{d}YIC(1)|[1-Me-Trp]QDW[Sar]AHRC(1)[N-Me-Ile]-NH2
(Compound 49)
(SEQ ID NO: 171)
Ac-EGSAIC(1)|[1-Me-Trp]QDWGEHRC(1)[Sar]E-NH2
(Compound 50)
(SEQ ID NO: 172)
Ac-EGSAIC(1)|[2-Nal]QDWGEHRC(1)[Sar]E-NH2
(Compound 51)
(SEQ ID NO: 173)
Ac-IC(1)|[1-Me-Trp]QDWGEHRC(1)TGAES-NH2
(Compound 52)
(SEQ ID NO: 174)
Ac-IC(1)|[2-Nal]QDWGEHRC(1)TGAES-NH2
(Compound 53)
(SEQ ID NO: 175)
Ac-EGSAFC(1)|[1-Me-Trp]QDWGEHRC(1)[Sar]E-NH2
(Compound 54)
(SEQ ID NO: 176)
Ac-EGSAYC(1)|[1-Me-Trp]QDWGEHRC(1)[Sar]E-NH2
(Compound 55)
(SEQ ID NO: 177)
Ac-EGSAIC(1)IWQDWGEHRC(1)TE-NH2
(Compound 56)
(SEQ ID NO: 178)
Ac-EGSAFC(1)|[1-Nal]QDWGEHRC(1)TE-NH2
(Compound 57)
(SEQ ID NO: 179)
Ac-EGSAFC(1)|[1-Me-Trp]QDWGEHRC(1)TE-NH2
(Compound 58)
(SEQ ID NO: 180)
Ac-EGSAFC(1)|[1-Me-Trp]QDWGEHRC(1)EGE-NH2
(Compound 59)
(SEQ ID NO: 181)
Ac-EGSAYC(1)|[1-Me-Trp]QDWGEHRC(1)TE-NH2
(Compound 60)
(SEQ ID NO: 182)
Ac-EGSAFC(1)|[2-Nal]QDWGEHRC(1)TE-NH2
(Compound 61)
(SEQ ID NO: 183)
Ac-FC(1)|[1-Me-Trp]QDWGEHRC(1)TGAES-NH2
(Compound 62)
(SEQ ID NO: 184)
Ac-YC(1)|[1-Me-Trp]QDWGEHRC(1)TGAES-NH2
(Compound 63)
(SEQ ID NO: 185)
Ac-FC(1)|[1-Nal]QDWGEHRC(1)TGAES-NH2
(Compound 64)
(SEQ ID NO: 186)
Ac-FC(1)|[2-Nal]QDWGEHRC(1)TGAES-NH2
(Compound 65)
(SEQ ID NO: 187)
Ac-YC(1)|[2-Nal]QDWGEHRC(1)TGAES-NH2
(Compound 66)
(SEQ ID NO: 188)
Ac-YC(1)IWQDWGEHRC(1)TGAES-NH2
(Compound 67)
(SEQ ID NO: 189)
Ac-SEFC(1)|[1-Me-Trp]QDWGEHRC(1)TGAES-NH2
(Compound 68)
(SEQ ID NO: 190)
Ac-YC(1)|[1-Me-Trp]QDWGEHRC(1)TEAGS-NH2
(Compound 69)
(SEQ ID NO: 191)
Ac-YC(1)|[1-Me-Trp]QDWGEHRC(1)TESGA-NH2
(Compound 70)
(SEQ ID NO: 192)
Ac-EGSAYC(1)|[1-Me-Trp]QEWGEHRC(1)[Sar]E-NH2
(Compound 71)
(SEQ ID NO: 193)
Ac-SEYC(1)|[1-Me-Trp]QDWGEHRC(1)[Sar]EA-NH2
(Compound 72)
(SEQ ID NO: 194)
Ac-FC(1)|[1-Me-Trp]QDW[Sar]EHRC(1)TGAES-NH2
(Compound 73)
(SEQ ID NO: 195)
H-{d}YFC(1)|[1-Me-Trp]QDW[Sar]EHRC(1)TGAES-NH2
(Compound 74)
(SEQ ID NO: 196)
Ac-SEFC(1)|[1-Me-Trp]QDWGEHRC(1)[Sar]GAES-NH2
(Compound 75)
(SEQ ID NO: 197)
Ac-SEFC(1)|[1-Me-Trp]QDWGEHRC(1)[Sar]EA-NH2
(Compound 76)
(SEQ ID NO: 198)
Ac-SEFC(1)|[1-Me-Trp]QDW[Sar]EHRC(1)[Sar]EA-NH2
(Compound 77)
(SEQ ID NO: 199)
Ac-SEFC(1)|[1-Me-Trp]QDW[Sar]EHRC(1)TEA-NH2
(Compound 78)
(SEQ ID NO: 200)
Ac-SEFC(1)|[1-Me-Trp]QDWGEHRC(1)[Sar]E-NH2
(Compound 79)
(SEQ ID NO: 201)
Ac-SEFC(1)|[1-Me-Trp]QDW[Sar]EHRC(1)[Sar]E-NH2
(Compound 80)
(SEQ ID NO: 202)
Ac-EFC(1)|[1-Me-Trp]QDWGEHRC(1)[Sar]EA-NH2
(Compound 81)
(SEQ ID NO: 203)
Ac-SE[Sar]C(1)|[1-Me-Trp]QDWGEHRC(1)[Sar]EA-NH2
(Compound 82)
(SEQ ID NO: 204)
Ac-SE[Sar]C(1)|[1-Me-Trp]QDWGEHRC(1)TEA-NH2
(Compound 83)
(SEQ ID NO: 205)
Ac-SEFC(1)|[1-Me-Trp]QEWGEHRC(1)[Sar]EA-NH2
(Compound 84)
(SEQ ID NO: 206)
Ac-SEFC(1)[1-Me-Trp]QDWGEHRC(1)SEA-NH2
(Compound 85)
(SEQ ID NO: 207)
Ac-EFC(1)|[1-Me-Trp]QDWGEHRC(1)ES-NH2
(Compound 86)
(SEQ ID NO: 208)
Ac-SEFC(1)|[1-Me-Trp]QDWGEHKC(1)[Sar]EA-NH2
(Compound 87)
(SEQ ID NO: 209)
Ac-GEFC(1)|[1-Me-Trp]QDWGEHRC(1)[Sar]EA-NH2
(Compound 88)
(SEQ ID NO: 210)
Ac-GE[Sar]C(1)|[1-Me-Trp]QDWGEHRC(1)TEA-NH2
(Compound 89)
(SEQ ID NO: 211)
Ac-SE[Sar]C(1)|[1-Me-Trp]QEW[Sar]EHRC(1)TEA-NH2
(Compound 90)
(SEQ ID NO: 212)
Ac-SE[Sar]C(1)|[1-Me-Trp]QEWGEHRC(1)[Sar]EA-NH2
(Compound 91)
(SEQ ID NO: 213)
H-{d}Y[Sar]C(1)|[1-Me-Trp]QDWGEHRC(1)TEA-NH2

Alternatively, the compstatin analogue may comprise one of the following sequences:[K*]GSAIC(1)IWQDWGEHRC(1)TEGE (Compound 100) (SEQ ID NO: 214)
ASGEYC(1)I[1-Me-Trp]QDWGEHRC(1)[Sar]EGE-[K*] (Compound 113) (SEQ ID NO: 215)
EFC(1)I[1-Me-Trp]QDWGEHRC(1)EGE-[K*] (Compound 134) (SEQ ID NO: 216)
EGSAIC(1)IWQDWGEHRC(1)TEG[K*] (Compound 101) (SEQ ID NO: 217)
EGSAYC(1)I[1-Me-Trp]QDWGEH[K*]C(1)[Sar]E (Compound 103) (SEQ ID NO: 218)
EGSAYC(1)I[1-Me-Trp]QDWGEHRC(1)[Sar]EG-[K*] (Compound 104) (SEQ ID NO: 219)
EGSAYC(1)I[1-Me-Trp]QDWGEHRC(1)[Sar]EGE-[K*] (Compound 109) (SEQ ID NO: 220)
EGSAYC(1)I[1-Me-Trp]QDWGEHRC(1)[Sar]EGK-[K*] (Compound 110) (SEQ ID NO: 221)
EGSAYC(1)I[1-Me-Trp]QDWGEHRC(1)[Sar]EK[γGlu]-[K*] (Compound 111) (SEQ ID NO: 222)
FC(1)I[1-Me-Trp]QDWGEHRC(1)TGAES-[K*] (Compound 102) (SEQ ID NO: 223)
IC(1)/WQDWGEHRC(1)TEG-[K*] (Compound 92) (SEQ ID NO: 224)
IC(1)IWQDWGEHRC(1)TEGE-[K*] (Compound 94) (SEQ ID NO: 225)
SAYC(1)I[1-Me-Trp]QDWGEHRC(1)[Sar]E-[K*] (Compound 105) (SEQ ID NO: 226)
SEFC(1)I[1-Me-Trp]QDWGEHRC(1)[Sar]EGA-[K*] (Compound 119) (SEQ ID NO: 227)
SEFC(1)I[1-Me-Trp]QDWGEHRC(1)[Sar]EGE[Peg3][Peg3]-[K*] (Compound 123) (SEQ ID NO: 228)
SEFC(1)I[1-Me-Trp]QDWGEHRC(1)[Sar]EGEGGG-[K*] (Compound 129) (SEQ ID NO: 229)
SEFC(1)I[1-Me-Trp]QDWGEHRC(1)[Sar]EGE[Peg3]-[K*] (Compound 138) (SEQ ID NO: 230)
SEFC(1)I[1-Me-Trp]QDWGEHRC(1)[Sar]EGE[Peg3]ES-[K*] (Compound 140) (SEQ ID NO: 231)
SEFC(1)I[1-Me-Trp]QDWGEHRC(1)[Sar]EGE[Peg3][Peg3]-[K*] (Compound 127) (SEQ ID NO: 232)
SEFC(1)I[1-Me-Trp]QDWGEHRC(1)[Sar]EGESES-[K*] (Compound 139) (SEQ ID NO: 233)
SEFC(1)I[1-Me-Trp]QDWGEHRC(1)[Sar]EK[γGlu]GGG-[K*] (Compound 132) (SEQ ID NO: 234)
SEFC(1)I[1-Me-Trp]QDWGEHRC(1)TEGE[8-aminooctanoyl]-[K*] (Compound 136) (SEQ ID NO: 235)
SEFC(1)I[1-Me-Trp]QDWGEHRC(1)TEGE[8-aminooctanoyl]E-[K*] (Compound 137) (SEQ ID NO: 236)
SEFC(1)I[1-Me-Trp]QDWGEHRC(1)TEGEGGG-[K*] (Compound 130) (SEQ ID NO: 237)
SEFC(1)I[1-Me-Trp]QDWGEHRC(1)TEGE[Peg3]ES-[K*] (Compound 142) (SEQ ID NO: 238)
SEFC(1)I[1-Me-Trp]QDWGEHRC(1)TEGE[Peg3][Peg3]-[K*] (Compound 126) (SEQ ID NO: 239)
SEFC(1)I[1-Me-Trp]QDWGEHRC(1)TEK[γGlu]GGG-[K*] (Compound 133) (SEQ ID NO: 240)
SEFC(1)I[1-Me-Trp]QDWGEHRC(1)TGAES-[K*] (Compound 135) (SEQ ID NO: 241)
SEFC(1)I[1-Me-Trp]QEWGEHRC(1)[Sar]EGA-[K*] (Compound 120) (SEQ ID NO: 242)
SEFC(1)I[1-Me-Trp]QEWGEHRC(1)[Sar]EGE[Peg3][Peg3]-[K*] (Compound 124) (SEQ ID NO: 243)
SEYC(1)I[1-Me-Trp]QDWGEHRC(1)[Sar]EGA-[K*] (Compound 112) (SEQ ID NO: 244)
SEYC(1)I[1-Me-Trp]QDWGEHRC(1)[Sar]EGE[Peg3][Peg3]-[K*] (Compound 117) (SEQ ID NO: 245)
SEYC(1)I[1-Me-Trp]QDWGEHRC(1)[Sar]EGE-[K*] (Compound 114) (SEQ ID NO: 246)
SEYC(1)I[1-Me-Trp]QEW[Sar]EHRC(1)[Sar]EK[γGlu]A-[K*] (Compound 121) (SEQ ID NO: 247)
SEYC(1)I[1-Me-Trp]QEWGEHRC(1)[Sar]EGA-[K*] (Compound 122) (SEQ ID NO: 248)
SEYC(1)I[1-Me-Trp]QEWGEHRC(1)[Sar]EGE[Peg3][Peg3]-[K*] (Compound 125) (SEQ ID NO: 249)
EGSEYC(1)I[1-Me-Trp]QDWGEHRC(1)[Sar]E (Compound 107) (SEQ ID NO: 250)
ESSAIC(1)IWQDWGEHRC(1)TEGE (Compound 99) (SEQ ID NO: 251)
SEFC(1)I[1-Me-Trp]QDWGEHRC(1)[Sar]EGE[Peg3][Peg3][Peg3]-[K*] (Compound 143) (SEQ ID NO: 252)
SEFC(1)I[1-Me-Trp]QDW[Sar]EHRC(1)[Sar]E[Peg3][Peg3]-[K*] (Compound 144) (SEQ ID NO: 253)
EFC(1)I[1-Me-Trp]QDWGEHRC(1)[Sar]EA[Peg3][Peg3]-[K″] (Compound 145) (SEQ ID NO: 254)

For example, the compstatin analogue may comprise one of the following sequences:

(Compound 100)
(SEQ ID NO: 214)
[K*]GSAIC(1)IWQDWGEHRC(1)TEGE
(Compound 113)
(SEQ ID NO: 215)
ASGEYC(1)I[1-Me-Trp]QDWGEHRC(1)[Sar]EGE-[K*]
(Compound 134)
(SEQ ID NO: 216)
EFC(1)|[1-Me-Trp]QDWGEHRC(1)EGE-[K*]
(Compound 101)
(SEQ ID NO: 217)
EGSAIC(1)IWQDWGEHRC(1)TEG[K*]
(Compound 103)
(SEQ ID NO: 218)
EGSAYC(1)[1-Me-Trp]QDWGEH[K*]C(1)[Sar]
(Compound 104)
(SEQ ID NO: 219)
EGSAYC(1)I[1-Me-Trp]QDWGEHRC(1)[Sar]EG-[K*]
(Compound 109)
(SEQ ID NO: 220)
EGSAYC(1)I[1-Me-Trp]QDWGEHRC(1)[Sar]EGE-[K*]
(Compound 110)
(SEQ ID NO: 221)
EGSAYC(1)I[1-Me-Trp]QDWGEHRC(1)[Sar]EGK-[K*]
(Compound 111)
(SEQ ID NO: 222)
EGSAYC(1)I[1-Me-Trp]QDWGEHRC(1)[Sar]EK[yGlu]-[K*]
(Compound 102)
(SEQ ID NO: 223)
FC(1)[1-Me-Trp]QDWGEHRC(1)TGAES-[K*]
(Compound 92)
(SEQ ID NO: 224)
IC(1)IWQDWGEHRC(1)TEG-[K*]
(Compound 94)
(SEQ ID NO: 225)
IC(1)IWQDWGEHRC(1)TEGE-[K*]
(Compound 105)
(SEQ ID NO: 226)
SAYC(1)I[1-Me-Trp]QDWGEHRC(1)[Sar]E-[K*]
(Compound 119)
(SEQ ID NO: 227)
SEFC(1)I[1-Me-Trp]QDWGEHRC(1)[Sar]EGA-[K*]
(Compound 123)
(SEQ ID NO: 228)
SEFC(1)I[1-Me-Trp]QDWGEHRC(1)[Sar]EGE[Peg3][Peg3]-
[K*]
(Compound 129)
(SEQ ID NO: 229)
SEFC(1)[1-Me-Trp]QDWGEHRC(1)[Sar]EGEGGG-[K*]
(Compound 138)
(SEQ ID NO: 230)
SEFC(1)I[1-Me-Trp]QDWGEHRC(1)[Sar]EGE[Peg3]-[K*]
(Compound 140)
(SEQ ID NO: 231)
SEFC(1)I[1-Me-Trp]QDWGEHRC(1)[Sar]EGE[Peg3]ES-[K*]
(Compound 127)
(SEQ ID NO: 232)
SEFC(1)I[1-Me-Trp]QDWGEHRC(1)[Sar]EGE[Peg3][Peg3]-
[K*]
(Compound 139)
(SEQ ID NO: 233)
SEFC(1)[1-Me-Trp]QDWGEHRC(1)[Sar]EGESES-[K*]
(Compound 132)
(SEQ ID NO: 234)
SEFC(1)I[1-Me-Trp]QDWGEHRC(1)[Sar]EK[yGlu]GGG-[K*]
(Compound 136)
(SEQ ID NO: 235)
SEFC(1)I[1-Me-Trp]QDWGEHRC(1)TEGE[8-aminooctanoyl]-
[K*]
(Compound 137)
(SEQ ID NO: 236)
SEFC(1)I[1-Me-Trp]QDWGEHRC(1)TEGE[8-aminooctanoyl]
E-[K*]
(Compound 130)
(SEQ ID NO: 237)
SEFC(1)I[1-Me-Trp]QDWGEHRC(1)TEGEGGG-[K*]
(Compound 142)
(SEQ ID NO: 238)
SEFC(1)I[1-Me-Trp]QDWGEHRC(1)TEGE[Peg3]ES-[K*]
(Compound 126)
(SEQ ID NO: 239)
SEFC(1) I[1-Me-Trp]QDWGEHRC(1)TEGE[Peg3][Peg3]-
[K*]
(Compound 133)
(SEQ ID NO: 240)
SEFC(1) I[1-Me-Trp]QDWGEHRC(1)TEK[yGlu]GGG-[K*]
(Compound 135)
(SEQ ID NO: 241)
SEFC(1) I[1-Me-Trp]QDWGEHRC(1)TGAES-[K*]
(Compound 120)
(SEQ ID NO: 242)
SEFC(1)[1-Me-Trp]QEWGEHRC(1)[Sar]EGA-[K*]
(Compound 124)
(SEQ ID NO: 243)
SEFC(1) I[1-Me-Trp]QEWGEHRC(1)[Sar]EGE[Peg3]
[Peg3]-[K*]
(Compound 112)
(SEQ ID NO: 244)
SEYC(1) I[1-Me-Trp]QDWGEHRC(1)[Sar]EGA-[K*]
(Compound 117)
(SEQ ID NO: 245)
SEYC(1) I[1-Me-Trp]QDWGEHRC(1)[Sar]EGE[Peg3]
[Peg3]-[K*]
(Compound 114)
(SEQ ID NO: 246)
SEYC(1) I[1-Me-Trp]QDWGEHRC(1)[Sar]EGE-[K*]
(Compound 121)
(SEQ ID NO: 247)
SEYC(1) I[1-Me-Trp]QEW[Sar]EHRC(1)[Sar]EK[yGlu]
A-[K*]
(Compound 122)
(SEQ ID NO: 248)
SEYC(1) I[1-Me-Trp]QEWGEHRC(1)[Sar]EGA-[K*]
(Compound 125)
(SEQ ID NO: 249)
SEYC(1) I[1-Me-Trp]QEWGEHRC(1)[Sar]EGE[Peg3]
[Peg3]-[K*]
(Compound 107)
(SEQ ID NO: 250)
EGSEYC(1) I[1-Me-Trp]QDWGEHRC(1)[Sar]E
(Compound 99)
(SEQ ID NO: 251)
ESSAIC(1)IWQDWGEHRC(1)TEGE
(Compound 143)
(SEQ ID NO: 252)
SEFC(1) I[1-Me-Trp]QDWGEHRC(1)[Sar]EGE[Peg3][Peg3]
[Peg3]-[K*]
(Compound 144)
(SEQ ID NO: 253)
SEFC(1) I[1-Me-Trp]QDW[Sar]EHRC(1)[Sar]E[Peg3]
[Peg3]-[K*]
(Compound 145)
(SEQ ID NO: 254)
EFC(1) I[1-Me-Trp]QDWGEHRC(1)[Sar]EA[Peg3][Peg3]-
[K*]

For example, the compstatin analogue may be:

(Compound 92)
(SEQ ID NO: 278)
Ac-IC(1)IWQDWGEHRC(1)TEG-K([15-carboxy-pentadecanoyl][Glu])-NH2
(Compound 93)
(SEQ ID NO: 279)
Ac-IC(1)IWQDWGEHRC(1) TEG-K([15-carboxy-pentadecanoyl][γGlu][Peg3][Peg3])-NH2
(Compound 94)
(SEQ ID NO: 283)
Ac-IC(1)IWQDWGEHRC(1)TEGE-K([15-carboxy-pentadecanoyl][γGlu][Peg3][Peg3])-NH2
(Compound 95)
(SEQ ID NO: 280)
Ac-IC(1)IWQDWGEHRC(1)TEG-K((15-carboxy-pentadecanoyl)-[(Piperazine-1-yl)-
acetyl][Peg3][Peg3])-NH2
(Compound 96)
(SEQ ID NO: 281)
Ac-IC(1)IWQDWGEHRC(1)TEG-K([17-carboxy-heptadecanoyl][γGlu][Peg3][Peg3])-NH2
(Compound 97)
(SEQ ID NO: 284)
Ac-IC(1)IWQDWGEHRC(1) TEGE-K([17-carboxy-heptadecanoyl][γGlu][Peg3][Peg3])-NH2
(Compound 98)
(SEQ ID NO: 282)
Ac-IC(1)IWQDWGEHRC(1) TEG-K([19-carboxy-nonadecanoyl][γGlu][Peg3][Peg3])-NH2
(Compound 99)
(SEQ ID NO: 318)
[15-Carboxy-pentadecanoyl]-ESSAIC(1)IWQDWGEHRC(1)TEGE-NH2
(Compound 100)
(SEQ ID NO: 268)
Ac-[K([15-carboxy-pentadecanoyl][γGlu][Peg3][Peg3])]-GSAIC(1)IWQDWGEHRC(1)
TEGE-NH2
(Compound 101)
(SEQ ID NO: 271)
Ac-EGSAIC(1)IWQDWGEHRC(1)TEG-K([15-carboxy-pentadecanoyl][γGlu])-NH2
(Compound 102)
(SEQ ID NO: 277)
Ac-FC(1)|[1-Me-Trp]QDWGEHRC(1)TGAES-K([15-carboxy-
pentadecanoyl][γGlu][Peg3][Peg3])-NH2
(Compound 103)
(SEQ ID NO: 272)
Ac-EGSAYC(1)|[1-Me-Trp]QDWGEH-K([15-carboxy-pentadecanoyl][γGlu][Peg3]
[Peg3])-C(1)[Sar]E-NH2
(Compound 104)
(SEQ ID NO: 273)
Ac-EGSAYC(1)|[1-Me-Trp]QDWGEHRC(1)[Sar]EG-K([15-carboxy-pentadecanoyl]
[γGlu][Peg3][Peg3])-NH2
(Compound 105)
(SEQ ID NO: 285)
Ac-SAYC(1)|[1-Me-Trp]QDWGEHRC(1)[Sar]E-K([17-carboxy-heptadecanoyl][γGlu]
KG[γGlu])-NH2
(Compound 106)
(SEQ ID NO: 286)
Ac-SAYC(1)|[1-Me-Trp]QDWGEHRC(1)[Sar]E-K([17-carboxy-heptadecanoyl]
[γGlu]G[γGlu])-NH2
(Compound 107)
(SEQ ID NO: 316)
[15-Carboxy-pentadecanoyl]-EGSEYC(1)|[1-Me-Trp]QDWGEHRC(1)[Sar]E-NH2
(Compound 108)
(SEQ ID NO: 317)
[17-Carboxy-heptadecanoyl]-EGSEYC(1)|[1-Me-Trp]QDWGEHRC(1)[Sar]E-NH2
(Compound 109)
(SEQ ID NO: 274)
Ac-EGSAYC(1)|[1-Me-Trp]QDWGEHRC(1)[Sar]EGE-K([17-carboxy-heptadecanoyl]
[γGlu]G[γGlu])-NH2
(Compound 110)
(SEQ ID NO: 275)
Ac-EGSAYC(1)|[1-Me-Trp]QDWGEHRC(1)[Sar]EGK-K([17-carboxy-heptadecanoyl]
[γGlu]G[γGlu])-NH2
(Compound 111)
(SEQ ID NO: 276)
Ac-EGSAYC(1)|[1-Me-Trp]QDWGEHRC(1)[Sar]EK([yGlu]-K([17-carboxy-heptadecanoyl]
[γGlu](peg3)(peg3))-NH2
(Compound 112)
(SEQ ID NO: 307)
Ac-SEYC(1)|[1-Me-Trp]QDWGEHRC(1)[Sar]EGA-K([17-carboxy-heptadecanoyl][γGlu]-G
[γGlu])-NH2
(Compound 113)
(SEQ ID NO: 269)
Ac-ASGEYC(1)|[1-Me-Trp]QDWGEHRC(1)[Sar]EGE-K([17-carboxy-heptadecanoyl][γGlu]-G
[γGlu])-NH2
(Compound 114)
(SEQ ID NO: 310)
Ac-SEYC(1)|[1-Me-Trp]QDWGEHRC(1)[Sar]EGE-K([17-carboxy-heptadecanoyl][γGlu]-G
[γGlu])-NH2
(Compound 115)
(SEQ ID NO: 311)
Ac-SEYC(1)|[1-Me-Trp]QDWGEHRC(1)[Sar]EGK-K([17-carboxy-heptadecanoyl][γGlu]-G
[γGlu])-NH2
(Compound 116)
(SEQ ID NO: 312)
Ac-SEYC(1)|[1-Me-Trp]QDWGEHRC(1)[Sar]EGE-K([17-carboxy-heptadecanoyl][γGlu]-K
[γGlu])-NH2
(Compound 117)
(SEQ ID NO: 309)
Ac-SEYC(1)|[1-Me-Trp]QDWGEHRC(1)[Sar]EGE[Peg3][Peg3]-K([17-carboxy-heptadecanoyl]
[yGlu]-G[γGlu])-NH2
(Compound 118)
(SEQ ID NO: 308)
Ac-SEYC(1)|[1-Me-Trp]QDWGEHRC(1)[Sar]EGA-K([17-carboxy-heptadecanoyl]-
[γGlu]G[Peg3][γGlu][Peg3])-NH2
(Compound 119)
(SEQ ID NO: 287)
Ac-SEFC(1)|[1-Me-Trp]QDWGEHRC(1)[Sar]EGA-K([17-carboxy-heptadecanoyl]-
[γGlu]G[Peg3][γGlu][Peg3])-NH2
(Compound 120)
(SEQ ID NO: 305)
Ac-SEFC(1)|[1-Me-Trp]QEWGEHRC(1)[Sar]EGA-K([17-carboxy-heptadecanoyl]-
[γGlu]G[Peg3][γGlu][Peg3])-NH2
(Compound 121)
(SEQ ID NO: 313)
Ac-SEYC(1)|[1-Me-Trp]QEW[Sar]EHRC(1)[Sar]EK[yGlu]A-K([17-carboxy-heptadecanoyl]-
[γGlu]G[Peg3][γGlu][Peg3])-NH2
(Compound 122)
(SEQ ID NO: 314)
Ac-SEYC(1)|[1-Me-Trp]QEWGEHRC(1)[Sar]EGA-K([17-carboxy-heptadecanoyl]-
[γGlu]G[Peg3][γGlu][Peg3])-NH2
(Compound 123)
(SEQ ID NO: 288)
Ac-SEFC(1)|[1-Me-Trp]QDWGEHRC(1)[Sar]EGE[Peg3][Peg3]-K([17-carboxy-heptadecanoyl]
[γGlu]G[γGlu])-NH2
(Compound 124)
(SEQ ID NO: 306)
Ac-SEFC(1)|[1-Me-Trp]QEWGEHRC(1)[Sar]EGE[Peg3[Peg3]-K([17-carboxy-heptadecanoyl]-
[γGlu]G[γGlu])-NH2
(Compound 125)
(SEQ ID NO: 315)
Ac-SEYC(1)|[1-Me-Trp]QEWGEHRC(1)[Sar]EGE[Peg3][Peg3]-K([17-carboxy-heptadecanoyl]
[γGlu]G[γGlu])-NH2
(Compound 126)
(SEQ ID NO: 302)
Ac-SEFC(1)|[1-Me-Trp]QDWGEHRC(1)TEGE[Peg3][Peg3]-K([17-carboxy-heptadecanoyl)
[γGlu]G[γGlu])-NH2
(Compound 127)
(SEQ ID NO: 292)
Ac-SEFC(1)|[1-Me-Trp]QDWGEHRC(1)[Sar]-EGE-[Peg3][Peg3]-K([15-carboxy-pentadecanoyl]
[γGlu]G[γGlu])-NH2
(Compound 128)
(SEQ ID NO: 293)
Ac-SEFC(1)|[1-Me-Trp]QDWGEHRC(1)[Sar]EGE[Peg3][Peg3]-K([19-carboxy-
nonadecanoyl][γGlu]G[γGlu])-NH2
(Compound 129)
(SEQ ID NO: 289)
Ac-SEFC(1)|[1-Me-Trp]QDWGEHRC(1)[Sar]EGEGGG-K([17-carboxy-heptadecanoyl]-
[γGlu]G[γGlu])-NH2
(Compound 130)
(SEQ ID NO: 299)
Ac-SEFC(1)|[1-Me-Trp]QDWGEHRC(1)TEGEGGG-K([17-carboxy-heptadecanoyl]-
[γGlu]G[γGlu])-NH2
(Compound 131)
(SEQ ID NO: 300)
Ac-SEFC(1)|[1-Me-Trp]-QDWGEHRC(1)TEGEGGG-K([15-carboxy-pentadecanoyl][γGlu]-G
[γGlu])-NH2
(Compound 132)
(SEQ ID NO: 296)
Ac-SEFC(1)|[1-Me-Trp]-QDWGEHRC(1)[Sar]EK[γGlu]GGG-K([17-carboxy-heptadecanoyl]
[γGlu]-G[γGlu])-NH2
(Compound 133)
(SEQ ID NO: 303)
Ac-SEFC(1)|[1-Me-Trp]QDWGEHRC(1)TEK[γGlu]GGG-K([17-carboxy-heptadecanoyl][γGlu]-
G[γGlu])-NH2
(Compound 134)
(SEQ ID NO: 270)
Ac-EFC(1)|[1-Me-Trp]QDWGEHRC(1)EGE-K([17-carboxy-heptadecanoyl][γGlu]G
[γGlu])-NH2
(Compound 135)
(SEQ ID NO: 304)
Ac-SEFC(1)|[1-Me-Trp]QDWGEHRC(1)TGAES-K([15-carboxy-hexadecanoyl][γGlu]
G[γGlu])-NH2
(Compound 136)
(SEQ ID NO: 297)
Ac-SEFC(1)|[1-Me-Trp]QDWGEHRC(1)TEGE[8-aminooctanoyl]-K([17-carboxy-
heptadecanoyl][γGlu]-G[γGlu])-NH2
(Compound 137)
(SEQ ID NO: 298)
Ac-SEFC(1)|[1-Me-Trp]QDWGEHRC(1)TEGE[8-aminooctanoyl]E-K([17-carboxy-
heptadecanoyl][γGlu]G[γGlu]])-NH2
(Compound 138)
(SEQ ID NO: 290)
Ac-SEFC(1)|[1-Me-Trp]QDWGEHRC(1)[Sar]EGE[Peg3]-K([17-carboxy-heptadecanoyl]-
[γGlu]G[γGlu])-NH2
(Compound 139)
(SEQ ID NO: 294)
Ac-SEFC(1)|[1-Me-Trp]QDWGEHRC(1)[Sar]EGESES-K([17-carboxy-heptadecanoyl]-
[γGlu]G[γGlu])-NH2
(Compound 140)
(SEQ ID NO: 291)
Ac-SEFC(1)|[1-Me-Trp]QDWGEHRC(1)[Sar]EGE[Peg3]ES-K([17-carboxy-heptadecanoyl]-
[γGlu]G[γGlu])-NH2
(Compound 141)
(SEQ ID NO: 295)
Ac-SEFC(1)|[1-Me-Trp]QDWGEHRC(1)[Sar]EGESES-K([17-carboxy-heptadecanoyl]
[γGlu])-NH2
(Compound 142)
(SEQ ID NO: 301)
Ac-SEFC(1)|[1-Me-Trp]QDWGEHRC(1)TEGE[Peg3]ES-K([17-carboxy-heptadecanoyl]
[γGlu])-NH2
(Compound 143)
(SEQ ID NO: 319)
Ac-SEFC(1)|[1-Me-Trp]QDWGEHR[C(1)[Sar]EGE[Peg3][Peg3][Peg3]-K([17-carboxy-
heptadecanoyl][γGlu]G[γGlu])-NH2
(Compound 144)
(SEQ ID NO: 320)
Ac-SEFC(1)|[1-Me-Trp]QDW[Sar]EHRC(1)[Sar]E[Peg3][Peg3]-K([17-carboxy-
heptadecanoyl][γGlu]G[γGlu])-NH2
(Compound 145)
(SEQ ID NO: 321)
Ac-EF[C(1)|[1-Me-Trp]QDWGEHRC(1)[Sar]EA[Peg3][Peg3]-K([17-carboxy-
heptadecanoyl][γGlu]G[γGlu])-NH2.

Compstatin analogues made in the prior art have been shown to possess improved activity as compared with the parent peptide, i.e., up to about 99-fold (Mallik, B. et al, 2005, supra; WO 2004/026328), and up to about 264-fold (Katragadda et al., 2006, supra; WO2007/062249).

In accordance with the present invention, information about the biological and physico-chemical characteristics of Ac-compstatin binding to C3 have been employed to design compstatin analogues with significantly improved activity compared to the parent compstatin analogues.

Preferably, the compstatin analogs have greater activity than Ac-compstatin, e.g. at least 10-fold greater activity, at least 20-fold greater activity, at least 30-fold greater activity than Ac-compstatin. In other embodiments, the analogs have at least 40-, 50-, 60-, 70-, 80-, 90-, 100-, 110-, 120-, 130-, 140-, 150-fold or greater activity than Ac-compstatin, as compared utilizing the assays described in the examples.

A compound of the invention typically has greater activity than an otherwise identical compound having valine instead of isoleucine at the position corresponding to Val3 of compstatin.

The compstatin analogues are capable of binding to C3 and/or C3b, and of inhbiting activation of the complement cascade, particularly downstream of C3, e.g. by inhibiting cleavage of C3 by C3 convertases.

The compstatin analogues are also typically capable of inhibiting complement-driven haemolysis. Complement-driven haemolysis is typically assessed (in a “haemolysis assay”) by contacting serum from a first mammalian species (e.g. human serum) with erythrocytes (red blood cells; RBC) from a second mammalian species (e.g. sheep or any other suitable species), typically in the presence of mammalian immunoglobulin capable of binding to the erythrocytes. Complement in the serum is activated by the cell-bound immunoglobulin, leading to lysis of the erythrocytes, i.e. haemolysis. The immunoglobulin may be from the first species, or may be from a third mammalian species as long as it is capable of activating complement from the first species.

In such an assay, a test compound will typically be pre-incubated with the serum before the serum is contacted with the erythroctes. The erythrocytes may also be pre-incubated with the immunoglobulin before contacting with the serum.

In the examples below, human serum is pre-incubated with a test compound, and sheep erythroctes are pre-incubated with rabbit anti-serum against sheep erythrocytes, before the serum and erythrocytes are combined.

Thus, the activity of the compstatin analogues may be determined with reference to one or more biological activities selected from (1) binding to C3 protein, (2) binding to C3b protein, (3) inhibiting the cleavage of native C3 by C3 convertases, and (4) inhibiting the activation of the complement system.

Thus a compstatin analogue of the invention may bind C3 or C3b with a higher affinity than that of compstatin. For example, they may have a Kd at least 10-fold lower, at least 20-fold lower, or at least 30-fold lower than Ac-compstatin, e.g. at least 40-, 50-, 60-, 70-, 80-, 90-, 100-, 110-, 120-, 130-, 140-, or 150-fold lower than Ac-compstatin. The Kd may be determined by surface plasmon resonance (SPR), e.g. using an assay as described in Example 4.

A compstatin analogue of the invention typically binds C3 or C3b with a greater affinity (i.e. a lower Kd) than that of an otherwise identical compound having valine instead of isoleucine at the position corresponding to Val3 of compstatin.

A compstatin analogue of the invention may have a greater ability to inhibit haemolysis than Ac-compstatin. For example, it may inhibit haemolysis with an IC₅₀ at least 10-fold, at least 20-fold, or at least 30-fold lower than Ac-compstatin, e.g. at least 40-, 50-, 60-, 70-, 80-, 90-, 100-, 110-, 120-, 130-, 140-, 150-, 200-, 250-, 300-350-, 400-, 450-, 500-fold lower than Ac-compstatin.

A compstatin analogue of the invention typically has a greater ability to inhibit haemolysis (i.e. a lower IC₅₀) than an otherwise identical compound having valine instead of isoleucine at the position corresponding to Val3 of compstatin.

Preferably, the in vitro effect of the compounds of the present invention are assessed by measuring their inhibitory effect on the classical complement pathway in a haemolysis assay, e.g. using the assay described in Example 2.

Compstatin analogues having acylation may have a lower absolute activity than an otherwise identical compound lacking acylation, but have additional benefits including prolonged in vivo half life which may offset any apparent reduction of absolute activity.

### Synthesis of Compstatin Analogues

It is preferred to synthesize compstatin analogues of the present invention by means of solid-phase or liquid-phase peptide synthesis methodology. In this context, reference may be made to WO 98/11125 and, among many others, Fields, G. B. et al., 2002, “Principles and practice of solid-phase peptide synthesis”. In: Synthetic Peptides (2nd Edition), and the Examples herein.

In accordance with the present invention, a compstatin analogue of the invention may be synthesized or produced in a number of ways, including for example, a method which comprises:
(a) synthesizing the compstatin analogues by means of solid-phase or liquid-phase peptide synthesis methodology and recovering the synthesized compstatin analogues thus obtained; or
(b) expressing a precursor peptide sequence from a nucleic acid construct that encodes the precursor peptide, recovering the expression product, and modifying the precursor peptide to yield a compound of the invention.

The precursor peptide may be modified by introduction of one or more non-proteinogenic amino acids, e.g. Aib, Orn, Dap, 1-Me-Trp, 1-Nal, 2-Nal, Sar, γGlu or Dab, or by the introduction of an appropriate terminal groups Y1 and/or Y2.

Expression is typically performed from a nucleic acid encoding the precursor peptide, which may be performed in a cell or a cell-free expression system comprising such a nucleic acid.

It is preferred to synthesize the analogues of the invention by means of solid-phase or liquid-phase peptide synthesis. In this context, reference is made to WO 98/11125 and, among many others, Fields, G B et al., 2002, “Principles and practice of solid-phase peptide synthesis”. In: Synthetic Peptides (2nd Edition), and the Examples herein.

For recombinant expression, the nucleic acid fragments encoding the precursor peptide will normally be inserted in suitable vectors to form cloning or expression vectors. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general outline, an expression vector comprises the following features in the 5′→3′ direction and in operable linkage: a promoter for driving expression of the nucleic acid fragment, optionally a nucleic acid sequence encoding a leader peptide enabling secretion (to the extracellular phase or, where applicable, into the periplasma), the nucleic acid fragment encoding the precursor peptide, and optionally a nucleic acid sequence encoding a terminator.

They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

The vectors of the invention are used to transform host cells to produce the precursor peptide. Such transformed cells can be cultured cells or cell lines used for propagation of the nucleic acid fragments and vectors, and/or used for recombinant production of the precursor peptides.

Preferred transformed cells are micro-organisms such as bacteria [such as the species Escherichia (e.g. E. coli), Bacillus (e.g. Bacillus subtilis), Salmonella, or Mycobacterium(preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment can be used for small-scale or large-scale preparation of the peptides of the invention.

When producing the precursor peptide by means of transformed cells, it is convenient, although far from essential, that the expression product is secreted into the culture medium.

### Medical Conditions

In a broad aspect, the present invention provides compstatin analogues of the present invention for use as a medicament or for use in therapy.

The compstatin analogues described herein have biological activities of binding to C3 protein and/or inhibiting complement activation. Generally, the compstatin analogues of the present invention may be used for the treatment or prevention conditions associated with excessive or unwanted activation of the complement system. Complement can be activated through three different pathways: the classical, lectin and alternative pathways. The major activation event that is shared by all three pathways is the proteolytic cleavage of the central protein of the complement system, C3, into its activation products C3a and C3b by C3 convertases.

Generation of these fragments leads to the opsonization of pathogenic cells by C3b and iC3b, a process that renders them susceptible to phagocytosis or clearance, and to the activation of immune cells through an interaction with complement receptors (Markiewski & Lambris, 2007, Am. J. Pathol., 171: 715-727). Deposition of C3b on target cells also induces the formation of new convertase complexes and thereby initiates a self-amplification loop. An ensemble of plasma and cell surface-bound proteins carefully regulates complement activation to prevent host cells from self-attack by the complement cascade. The 13 amino acid cyclic tridecapeptide used as a reference point for the design of the compstatin analogues of the present invention inhibits complement activation by binding to C3 and/or C3b, preventing the cleavage of native C3 by the C3 convertases. Without wishing to be bound by any particular theory, the present inventors believe that the compstatin analogues of the present invention also function in this way and may share one or more biological activities selected from (1) binding to C3 protein, (2) binding to C3b protein, (3) inhibiting the cleavage of native C3 by C3 convertases, and/or (4) inhibiting the activation of the complement system. The biological activity of the compstatin analogues of the present invention may be determined in vitro by measuring their inhibitory effect of the classical complement pathway in a haemolysis assay, for example using a protocol set out in the examples below.

Excessive activation or inappropriate regulation of complement can lead to a number of pathologic conditions, ranging from autoimmune diseases to inflammatory diseases (Holers, 2003, Clin. Immunol., 107: 140-51; Markiewski & Lambris, 2007, supra; Ricklin & Lambris, 2007, Nat. Biotechnol., 25: 1265-75; Sahu et al., 2000, J. Immunol., 165: 2491-9). These conditions include: (1) inhibiting complement activation to facilitate treatment of diseases or conditions including age-related macular degeneration, Stargardt disease, periodontitis, diabetic retinopathy, glaucoma, uveitis, rheumatoid arthritis, spinal cord injury, stroke, multiple sclerosis, Parkinson's disease, Alzheimer's disease, cancer, and respiratory disorders such as asthma, chronic obstructive pulmonary disease (COPD), allergic inflammation, emphysema, bronchitis, bronchiecstasis, cystic fibrosis, tuberculosis, pneumonia, respiratory distress syndrome (RDS -neonatal and adult), rhinitis and sinusitis; bacterial infections such as sepsis, ischemia-reperfusion injury in various tissues, myocardial infarction, anaphylaxis, paroxysmal nocturnal hemoglobinuria, autoimmune hemolytic anemias, psoriasis, hidradentitis suppurativa, myasthenia gravis, systemic lupus erythematosus, CHAPLE syndrome, C3 glomeropathy, IgA nephropathy, atypical hemolytic uremic syndrome, Crohn's disease, ulcerative colitis, antiphospholipid syndrome, or (2) inhibiting complement activation that occurs during cell or solid organ transplantation, or in the use of artificial organs or implants (e.g., by coating or otherwise treating the cells, organs, artificial organs or implants with a peptide of the invention); or (3) inhibiting complement activation that occurs during extracorporeal shunting of physiological fluids (blood, urine) (e.g., by coating the tubing through which the fluids are shunted with a compstatin analogue of the present invention).

### Pharmaceutical Compositions and Administration

In a further aspect, the present invention relates to a composition comprising a compstatin analogue according to the invention, or a pharmaceutically acceptable salt or solvate thereof, together with a carrier. In one embodiment of the invention, the composition is a pharmaceutical composition and the carrier is a pharmaceutically acceptable carrier. The present invention also relates to a pharmaceutical composition comprising a compstatin analogue according to the invention, or a salt and/or solvate thereof, together with a carrier, excipient or vehicle. Accordingly, the compstatin analogue of the present invention, or salts or solvates thereof, especially pharmaceutically acceptable salts and/or solvates thereof, may be formulated as compositions or pharmaceutical compositions prepared for storage or administration, and which comprise a therapeutically effective amount of a compstatin analogue of the present invention, or a salt or solvate thereof.

Suitable salts formed with bases include metal salts, such as alkali metal or alkaline earth metal salts.

In one embodiment, a pharmaceutical composition of the invention is one wherein the compstatin analogue is in the form of a pharmaceutically acceptable acid addition salt.

As will be apparent to one skilled in the medical art, a “therapeutically effective amount” of a compstatin analogue compound or pharmaceutical composition thereof of the present invention will vary depending upon, inter alia, the age, weight and/or gender of the subject (patient) to be treated. Other factors that may be of relevance include the physical characteristics of the specific patient under consideration, the patient's diet, the nature of any concurrent medication, the particular compound(s) employed, the particular mode of administration, the desired pharmacological effect(s) and the particular therapeutic indication. Because these factors and their relationship in determining this amount are well known in the medical arts, the determination of therapeutically effective dosage levels, the amount necessary to achieve the desired result of treating and/or preventing and/or remedying malabsorption and/or low-grade inflammation described herein, as well as other medical indications disclosed herein, will be within the ambit of the skilled person.

As used herein, the term “a therapeutically effective amount” refers to an amount which reduces symptoms of a given condition or pathology, and preferably which normalizes physiological responses in an individual with that condition or pathology. Reduction of symptoms or normalization of physiological responses can be determined using methods routine in the art and may vary with a given condition or pathology. In one aspect, a therapeutically effective amount of one or more compstatin analogues, or pharmaceutical compositions thereof, is an amount which restores a measurable physiological parameter to substantially the same value (preferably to within 30%, more preferably to within 20%, and still more preferably to within 10% of the value) of the parameter in an individual without the condition or pathology in question.

In one embodiment of the invention, administration of a compound or pharmaceutical composition of the present invention is commenced at lower dosage levels, with dosage levels being increased until the desired effect of preventing/treating the relevant medical indication is achieved. This would define a therapeutically effective amount. For the compstatin analogues of the present invention, alone or as part of a pharmaceutical composition, such human doses of the active compstatin analogue may be between about 0.01 pmol/kg and 500 μmol/kg body weight, between about 0.01 pmol/kg and 300 μmol/kg body weight, between 0.01 pmol/kg and 100 μmol/kg body weight, between 0.1 pmol/kg and 50 μmol/kg body weight, between 1 pmol/kg and 10 μmol/kg body weight, between 5 pmol/kg and 5 μmol/kg body weight, between 10 pmol/kg and 1 μmol/kg body weight, between 50 pmol/kg and 0.1 μmol/kg body weight, between 100 pmol/kg and 0.01 μmol/kg body weight, between 0.001 umol/kg and 0.5 μmol/kg body weight, between 0.05 μmol/kg and 0.1 μmol/kg body weight.

The therapeutic dosing and regimen most appropriate for patient treatment will of course vary with the disease or condition to be treated, and according to the patient's weight and other parameters. Without wishing to be bound by any particular theory, it is expected that doses, in the mg/kg range, and shorter or longer duration or frequency of treatment may produce therapeutically useful results, such as a statistically significant inhibition of the alternative and classical complement pathways. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject.

For local delivery to the eye, the pharmaceutically acceptable compositions may be formulated in isotonic, pH adjusted sterile saline or water, either with or without a preservative such as benzylalkonium chloride. Alternatively, for ophthalmic uses, the pharmaceutically acceptable compositions may be formulated in an ointment such as petrolatum or as eyedrops. Methods of local administration to the eye include, e.g., choroidal injection, transscleral injection or placing a scleral patch, selective arterial catheterization, eyedrops or eye ointments, intraocular administration including transretinal , subconjunctival bulbar, intravitreous injection, suprachoroidal injection, subtenon injection, scleral pocket and scleral cutdown injection, by osmotic pump, etc. The agent can also be alternatively administered intravascularly, such as intravenously (IV) or intraarterially. In choroidal injection and scleral patching, the clinician uses a local approach to the eye after initiation of appropriate anesthesia, including painkillers and ophthalmoplegics. A needle containing the therapeutic compound is directed into the subject's choroid or sclera and inserted under sterile conditions. When the needle is properly positioned the compound is injected into either or both of the choroid or sclera. When using either of these methods, the clinician can choose a sustained release or longer acting formulation. Thus, the procedure can be repeated only every several months or several years, depending on the subject's tolerance of the treatment and response.

The following examples are provided to describe the invention in greater detail. They are intended to illustrate, not to limit, the invention.

### EXAMPLE 1: SYNTHESIS OF COMPSTATIN ANALOGUES

### General Peptide Synthesis

List of abbreviations and suppliers

| | Abbreviation | Name | Brand/Supplier |
|---|---|---|---|
| Resins | | | |
| | | TentaGel ™ PHB AA(Proct)- | Rapp Polymere |
| | | Fmoc | |
| | | TentaGel ™ SRAM | Rapp Polymere |
| Amino acids | | | |
| | | Pseudoprolines (E.g. YS, | Jupiter Bioscience Ltd. |
| | | FS, FT) | |
| | | Fmoc-L-Aaa-OH | Senn Chemicals AG |
| Coupling reagents | | | |
| | Oxyma Pure | Ethyl cyanoglyoxylate-2- | Chem Impex international |
| | | oxime | |
| | DIC | Diisopropylcarbodiimide | Fluka/Sigma Aldrich Co. |
| | HATU | N-[(dimethylamino)-1H- | ChemPep Inc. |
| | | 1,2,3-triazol[4,5-b]pyridine- | |
| | | 1-ylmethylene]-N- | |
| | | methylmethanaminium | |
| | | hexafluorophosphate N- | |
| | | oxide | |
| | HOBt | Hydroxybenzotriazole | Sigma-Aldrich Co. |
| Solvents and reagents | | | |
| | Boc₂O | Di-tert-butyl pyrocarbonate | Advanced ChemTech |
| | DCM | Dichloromethane | Prolabo (VWR) |
| | DIPEA | Diisopropylethylamine | Fluka/Sigma Aldrich Co. |
| | DMF | N,N-dimethylformamide | Taminco |
| | Et₂O | Diethyl ether | Prolabo (VWR) |
| | EtOH | Ethanol | CCS Healthcare AB |
| | HCOOH | Formic acid (HPLC grade) | Sigma-Aldrich Co. |
| | H₂O | Water, Milli-Q water | Millipore |
| | MeCN | Acetonitrile (HPLC) | Sigma-Aldrich Co. |
| | NMP | N-methylpyrrolidone | Sigma-Aldrich Co. |
| | | Piperidine | Jubliant Life Sciences Ltd. |
| | TFA | Trifluoroacetic acid (HPLC) | Chemicals Raw Materials |
| | | | Ltd. |
| | TIS | Triisopropylsilane | Sigma-Aldrich Co. |
| | DODT | 2,2′- | Sigma-Aldrich Co |
| | | (ethylenedioxy)diethanethiol | |
| Other | MeOH | Methanol | Sigma-Aldrich Co. |
| | | Ascorbic acid | Sigma-Aldrich Co. |
| | I₂ | Iodine | Sigma-Aldrich Co |

### Apparatus and Synthetic Strategy

Peptides were synthesized batchwise on a peptide synthesiser, such as a CEM Liberty Peptide Synthesizer or a Symphony X Synthesizer, according to solid phase peptide synthetic procedures using 9-fluorenylmethyloxycarbonyl (Fmoc) as N-α-amino protecting group and suitable common protection groups for side-chain functionalities.

As polymeric support based resins, such as e.g. TentaGel™, was used. The synthesizer was loaded with resin that prior to usage was swelled in DMF.

### Coupling

### CEM Liberty Peptide Synthesizer

A solution of Fmoc-protected amino acid (4 equiv.) was added to the resin together with a coupling reagent solution (4 equiv.) and a solution of base (8 equiv.). The mixture was either heated by the microwave unit to 70-75° C. and coupled for 5 minutes or coupled with no heat for 60 minutes. During the coupling nitrogen was bubbled through the mixture.

### Symphony X Synthesizer

The coupling solutions were transferred to the reaction vessels in the following order: amino acid (4 equiv.), HATU (4 equiv.) and DIPEA (8 equiv.). The coupling time was 10 min at room temperature (RT) unless otherwise stated. The resin was washed with DMF (5×0.5 min). In case of repeated couplings the coupling time was in all cases 45 min at RT.

### Deprotection

### CEM Liberty Peptide Synthesizer

The Fmoc group was deprotected using piperidine in DMF or other suitable solvents. The deprotection solution was added to the reaction vessel and the mixture was heated for 30 sec. reaching approx. 40° C. The reaction vessel was drained and fresh deprotection solution was added and subsequently heated to 70-75° C. for 3 min. After draining the reaction vessel the resin was washed with DMF or other suitable solvents.

### Symphony X Synthesizer

Fmoc deprotection was performed for 2.5 minutes using 40% piperidine in DMF and repeated using the same conditions. The resin was washed with DMF (5×0.5 min).

### Side Chain Acylation

Fmoc-Lys(Dde)-OH or alternatively another amino acid with an orthogonal side chain protective group was introduced at the position of the acylation (side-chain lipidation). The N-terminal of the linier peptide was protected with Ac or Boc. While the peptide was still attached to the resin, the orthogonal side chain protective group was selectively cleaved using freshly prepared hydrazine hydrate (2-4%) in NMP for 2×15 min. The unprotected lysine side chain was then elongated using standard coupling conditions and Fmoc-deprotections with the desired building block. The lipidation moiety was coupled as the last step.

### Cleavage

The dried peptide resin was treated with TFA and suitable scavengers for approximately 2 hours. The volume of the filtrate was reduced and the crude peptide was precipitated after addition of diethylether. The crude peptide precipitate was washed several times with diethylether and finally dried.

### HPLC Purification of the Crude Peptide

The crude peptide was purified by preparative reverse phase HPLC using a conventional HPLC apparatus, such as a Gilson GX-281 with 331/332 pump combination, for binary gradient application equipped with a column, such as 5×25 cm Gemini NX 5u C18 110A column, and a fraction collector using a flow 20-40 ml/min with a suitable gradient of buffer A (0.1% Fomic acid, aq.) or A (0.1% TFA, aq.) and buffer B (0.1% Formic acid, 90% MeCN, aq.) or B (0.1% TFA, 90% MeCN, aq.). Fractions were analyzed by analytical HPLC and MS and selected fractions were pooled and lyophilized. The final product was characterized by HPLC and MS.

### Oxidation

Following purification and lyophilisation of the crude linear peptide, the peptide was redissolved in 0.1% TFA in water, acetonitrile and acetic acids until a clear solution. The concentration of the peptide solution was kept at approx. 1-2 mg/ml depending on the peptides ability to solubilize. The peptide solution was stirred, while a solution of iodine in methanol (approx. 1.5 equiv.) was added drop-wise until the peptide solution obtain an orange colour. After 10-15 minutes, the oxidation was finished and excess iodine was reduced with a solution of ascorbic acid in water (1 equiv.) until a colourless peptide solution. The peptide solution was diluted with water before preparative HPLC purification.

### Analytical HPLC

Final purities were determined by analytic HPLC (Agilent 1100/1200 series) equipped with auto sampler, degasser, 20 μl flow cell and Chromeleon software. The HPLC was operated with a flow of 1.2 ml/min at 40° C. using an analytical column, such as Kinetex 2.6 μm XB-C18 100A 100×8.6 mm column. The compound was detected and quantified at 215 nm. Buffers A (0.1% TFA, aq.) and buffer B (0.1% TFA, 90% MeCN, aq.).

### Mass Spectroscopy

Final MS analysis were determined on a conventional mass spectroscopy, e.g. Waters Xevo G2 TOF, equipped with electrospray detector with lock-mass calibration and MassLynx software. It was operated in positive mode using direct injection and a cone voltage of 15V (1 TOF), 30 V (2 TOF) or 45 V (3 TOF) as specified on the chomatogram. Precision was 5 ppm with a typical resolution of 15,000-20,000.

### Synthesis of Compound No 24

(SEQ ID NO: 146)
Ac-IC(1)IWQDWGEHRC(1)TEGE-NH₂

Solid phase peptide synthesis was performed on a Symphony X Synthesizer using standard Fmoc chemistry. TentaGel S RAM (2,51 g; 0.23 mmol/g) was swelled in DMF (20 ml) prior to use and the Fmoc-group was deprotected according to the procedure described above.

### Coupling

Suitable protected Fmoc-amino acids according to the sequence were coupled as described above using HATU as coupling reagent. All couplings were performed at R.T.

### Deprotection

Fmoc deprotection was performed according to the procedure described above.

### Cleavage of the Peptide from the Solid Support

The peptide-resin was washed with EtOH (3×10 ml) and Et2O (3×10 ml) and dried to constant weight at room temperature (r.t.). The peptide was cleaved from the resin by treatment with TFA/DODT (95/5; 60 ml, 2 h; r.t). The volume of the filtrate was reduced and the crude peptide was precipitated after addition of diethylether. The crude peptide precipitate was washed several times with diethylether and finally dried to constant weight at room temperature yield 760 mg crude peptide product (purity ˜30%).

### HPLC Purification of the Crude Linear Peptide

The crude peptide was purified by preparative reverse phase HPLC using a Gilson GX-281 with 331/332 pump combination for binary gradient application equipped with a 5×25 cm Gemini NX 5u C18 110A, column and a fraction collector and run at 35 ml/min with a gradient of buffer A (0.1% TFA, aq.) and buffer B (0.1% TFA, 90% MeCN, aq.) gradient from 20% B to 45% B in 47 min. Fractions were analyzed by analytical HPLC and MS and relevant fractions were pooled and lyophilized to yield 190 mg, with a purity of 85% as characterized by HPLC and MS as described above. Calculated monoisotopic MW=2001.58 found 2001.81.

### Oxidation of the Crude Linear Peptide

The 190 mg purified linear peptide was dissolved in 220 ml 0.1% TFA in water (65%) and acetonitrile (35%) until a clear solution. The peptide solution was stirred, while a solution of iodine in methanol (2.2 mL, approx. 1.5 equiv. iodine) was added drop-wise until the peptide solution obtain an orange colour. The reaction was followed by analytic HPLC but already after 10-15 minutes, the oxidation was finished. Excess iodine was reduced with a solution of ascorbic acid in water (220 μL, approx. 1 equiv.) until a colourless peptide solution. The peptide solution was reduced slightly by rota evaporation before purification on preparative HPLC.

### HPLC Purification of the Oxidized Peptide

The crude peptide was purified by preparative reverse phase HPLC using a Gilson GX-281 with 331/332 pump combination for binary gradient application equipped with a 5×25 cm Gemini NX 5u C18 110A, column and a fraction collector and run at 35 ml/min with a gradient of buffer A (0.1% TFA, aq.) and buffer B (0.1% TFA, 90% MeCN, aq.) gradient from 20% B to 45% B in 47 min. Fractions were analyzed by analytical HPLC and MS and relevant fractions were pooled and lyophilized to yield 138 mg, with a purity of 92% as characterized by HPLC and MS as described above. Calculated monoisotopic MW=1999.83 found 1999.54.

### Synthesis of Compound No 119

(SEQ ID NO: 287)
Ac-SEFC(1)|[1-Me-Trp]QDWGEHRC(1)[Sar]EGA-K([17-car-
boxy-heptadecanoyl][yGlu]G[Peg3][yGlu][Peg3])-NH₂

Solid phase peptide synthesis was performed on a Symphony X Synthesizer using standard Fmoc chemistry. TentaGel S RAM (3×˜1.3 g; 0.22 mmol/g) was swelled in DMF (3×10 ml) prior to use and the Fmoc-group was deprotected according to the procedure described above.

### Coupling

Suitable protected Fmoc-amino acids according to the sequence were coupled as described above using HATU as coupling reagent. All couplings were performed at R.T. The lysine used for the incorporation of the branched moiety was incorporated as Fmoc-Lys(Dde)-OH for orthogonal coupling

### Deprotection

Fmoc deprotection was performed according to the procedure described above.

### Side Chain Acylation

While the peptide was still attached to the resin, the orthogonal side-chain protective group (Dde) was selectively cleaved using freshly prepared hydrazine hydrate (2-4%) in NMP for 2×15 min. The unprotected lysine side-chain was doubled coupled with Fmoc-Peg3-OH followed by single couplings with Fmoc-Glu-OtBu, Fmoc-Peg3-OH, Fmoc-Gly-OH, Fmoc-Glu-OtBu and lastly the fatty acid moiety 17-carboxy-heptadecanoic acid mono tert-butyl ester using standard coupling conditions.

### Cleavage of the Peptide from the Solid Support

The peptide-resin was washed with EtOH (3×15 ml) and Et2O (3×150 ml) and dried to constant weight at room temperature (r.t.). The peptide was cleaved from the resin by treatment with TFA/DODT (95/5; 120 ml, 2 h; r.t.). The volume of the filtrate was reduced and the crude peptide was precipitated after addition of diethylether. The crude peptide precipitate was washed several times with diethylether and finally dried to constant weight at room temperature yield 2.36 g crude peptide product (purity ˜41-48%).

### HPLC Purification of the Crude Linear Peptide

The crude peptide was purified by preparative reverse phase HPLC using a Gilson GX-281 with 331/332 pump combination for binary gradient application equipped with a 5×25 cm Gemini NX 5u C18 110A, column and a fraction collector and run at 35 ml/min with a gradient of buffer A (0.1% TFA, aq.) and buffer B (0.1% TFA, 90% MeCN, aq.) gradient from 30% B to 60% B in 47 min. Fractions were analyzed by analytical HPLC and MS and relevant fractions were pooled and lyophilized to yield 744 mg, with a purity of 84% as characterized by HPLC and MS as described above. Calculated monoisotopic MW=3207.47 found 3207.32.

### Oxidation of the Crude Linear Peptide

The 744 mg purified linear peptide was dissolved in 350 ml 0.1% TFA in water, 150 ml acetonitrile and 100 ml acetic acid until a clear solution (total volume 600 ml). The peptide solution was stirred, while a solution of iodine in methanol (4.7 mL, approx. 1.5 equiv. iodine) was added drop-wise until the peptide solution obtain an orange colour. The reaction was followed by analytic HPLC but already after 10-15 minutes, the oxidation was finished. Excess iodine was reduced with a solution of ascorbic acid in water (150 μL, approx. 1 equiv.) until a colourless peptide solution. The peptide solution was reduced slightly by rota evaporation before purification on preparative HPLC.

### HPLC Purification of the Oxidized Peptide

The crude peptide was purified by preparative reverse phase HPLC using a Gilson GX-281 with 331/332 pump combination for binary gradient application equipped with a 5×25 cm Gemini NX 5u C18 110A, column and a fraction collector and run at 35 ml/min with a gradient of buffer A (0.1% TFA, aq.) and buffer B (0.1% TFA, 90% MeCN, aq.) gradient from 30% B to 60% B in 47 min. Fractions were analysed by analytical HPLC and MS and relevant fractions were pooled and lyophilized to yield 510 mg, with a purity of 91% as characterized by HPLC and MS as described above. Calculated monoisotopic MW=3205.47 found 3205.23.

TABLE 1
Synthesized compounds:

| Compound | Sequence |
|---|---|
| Compstatin 1-13 | H-IC(1)VVQDWGHHRC(1)T-NH2 (SEQ ID NO: 1) |
| Ac-compstatin | Ac-IC(1)VVQDWGHHRC(1)T-NH2 (SEQ ID NO: 1) |
| 4W9A* | Ac-IC(1)VWQDWGAHRC(1)T-NH2 (SEQ ID NO: 255) |
| Cp40* | H-{d}YIC(1)V[1-Me-Trp]QDW[Sar]AHRC(1)[N-Me-Ile]-NH2 |
| | (SEQ ID NO: 256) |
| A | Ac-IC(1)VWQDWGEHRC(1)T-NH2 (SEQ ID NO: 257) |
| B | Ac-IC(1)VWQDWGSHRC(1)T-NH2 (SEQ ID NO: 258) |
| C | Ac-ESSAIC(1)VWQDWGEHRC(1)T-NH2 (SEQ ID NO: 259) |
| D | Ac-IC(1)VWQDWGEHRC(1)TGAES-NH2 (SEQ ID NO: 260) |
| E | Ac-IC(1)VWQDWGAHSC(1)T-NH2 (SEQ ID NO: 261) |
| F | Ac-IC(1)VWQDWGEHSC(1)T-NH2 (SEQ ID NO: 262) |
| G | Ac-IC(1)VWQDWGEHRC(1)S-NH2 (SEQ ID NO: 263) |
| H | Ac-EGSAIC(1)VWQDWGEHRC(1)[Sar]E-NH2 (SEQ ID NO: 264) |
| J | Ac-IC(1)VWQDWGEHRC(1)TEGE-NH2 (SEQ ID NO: 265) |
| 1 | Ac-IC(1)IWQDWGAHRC(1)T-NH2 (SEQ ID NO: 35) |
| 2 | Ac-IC(1)IWQDWGEHRC(1)T-NH2 (SEQ ID NO: 41) |
| 3 | Ac-ESSAIC(1)IWQDWGEHRC(1)T-NH2 (SEQ ID NO: 140) |
| 4 | Ac-IC(1)I[1-Me-Trp]QDWGEHRC(1)T-NH2 (SEQ ID NO: 32) |
| 5 | Ac-IC(1)IWQDWGKHRC(1)T-NH2 (SEQ ID NO: 43) |
| 6 | Ac-IC(1)IWQDWGSHRC(1)T-NH2 (SEQ ID NO: 45) |
| 7 | Ac-IC(1)IWQKWGEHRC(1)T-NH2 (SEQ ID NO: 48) |
| 8 | Ac-IC(1)IWQKWGAHRC(1)TGAES-NH2 (SEQ ID NO: 142) |
| 9 | Ac-YC(1)IWQDWGEHRC(1)T-NH2 (SEQ ID NO: 53) |
| 10 | Ac-ESSAYC(1)IWQDWGEHRC(1)T-NH2 (SEQ ID NO: 143) |
| 11 | Ac-[Sar]C(1)IWQDWGEHRC(1)T-NH2 (SEQ ID NO: 17) |
| 12 | Ac-IC(1)IWQDWGAHRC(1)E-NH2 (SEQ ID NO: 34) |
| 13 | Ac-IC(1)IWQDWGEHRC(1)[Sar]-NH2 (SEQ ID NO: 38) |
| 14 | Ac-ESSAIC(1)IWQDWGEHRC(1)TGAES-NH2 (SEQ ID NO: 144) |
| 15 | Ac-IC(1)IWQDWGEHRC(1)TGAES-NH2 (SEQ ID NO: 145) |
| 16 | Ac-IC(1)IWQEWGEHRC(1)T-NH2 (SEQ ID NO: 46) |
| 17 | Ac-IC(1)IWQDWGDHRC(1)T-NH2 (SEQ ID NO: 37) |
| 18 | Ac-IC(1)IWQDWGRHRC(1)T-NH2 (SEQ ID NO: 44) |
| 19 | Ac-IC(1)IWQDWGAHSC(1)T-NH2 (SEQ ID NO: 36) |
| 20 | Ac-IC(1)IWQDWGEHSC(1)T-NH2 (SEQ ID NO: 42) |
| 21 | Ac-IC(1)IWQDWGEHRC(1)S-NH2 (SEQ ID NO: 40) |
| 22 | Ac-IC(1)IWQDWGEHRC(1)E-NH2 (SEQ ID NO: 39) |
| 23 | Ac-FC(1)IWQDWGEHRC(1)T-NH2 (SEQ ID NO: 29) |
| 24 | Ac-IC(1)IWQDWGEHRC(1)TEGE-NH2 (SEQ ID NO: 146) |
| 25 | Ac-IC(1)IWQDWGEHRC(1)TEA-NH2 (SEQ ID NO: 147) |
| 26 | Ac-IC(1)IWQDWGEHRC(1)TE-NH2 (SEQ ID NO: 148) |
| 27 | Ac-IC(1)IWQDWGEHRC(1)EGE-NH2 (SEQ ID NO: 149) |
| 28 | Ac-EGSAIC(1)IWQDWGEHRC(1)[Sar]E-NH2 (SEQ ID NO: 150) |
| 29 | Ac-EGSAIC(1)IWQDWGEHRC(1)T-NH2 (SEQ ID NO: 151) |
| 30 | Ac-EGEIC(1)IWQDWGEHRC(1)T-NH2 (SEQ ID NO: 152) |
| 31 | Ac-ESEIC(1)IWQDWGEHRC(1)T-NH2 (SEQ ID NO: 153) |
| 32 | Ac-SEIC(1)IWQDWGEHRC(1)TEA-NH2 (SEQ ID NO: 154) |
| 33 | Ac-EIC(1)IWQDWGEHRC(1)TE-NH2 (SEQ ID NO: 155) |
| 34 | Ac-EIC(1)IWQDWGEHRC(1)TEGE-NH2 (SEQ ID NO: 156) |
| 35 | Ac-EGEIC(1)IWQDWGEHRC(1)EGE-NH2 (SEQ ID NO: 157) |
| 36 | Ac-ESEIC(1)IWQDWGEHRC(1)EGE-NH2 (SEQ ID NO: 158) |
| 37 | Ac-KEKIC(1)IWQDWGEHRC(1)TEKE-NH2 (SEQ ID NO: 159) |
| 38 | Ac-EKGIC(1)IWQDWGEHRC(1)TEKP-NH2 (SEQ ID NO: 160) |
| 39 | Ac-IC(1)IWQDWGEHRC(1)TEGK-NH2 (SEQ ID NO: 161) |
| 40 | Ac-GSAIC(1)IWQDWGEHRC(1)[Sar]E-NH2 (SEQ ID NO: 162) |
| 41 | Ac-SAIC(1)IWQDWGEHRC(1)[Sar]E-NH2 (SEQ ID NO: 163) |
| 42 | Ac-SAIC(1)IWQDWGEHRC(1)TEG-NH2 (SEQ ID NO: 164) |
| 43 | Ac-FC(1)IWQDWGEHRC(1)TGAE-NH2 (SEQ ID NO: 165) |
| 44 | Ac-EGSAIC(1)IWQDWGEHRC(1)[Sar]EGE-NH2 (SEQ ID NO: 166) |
| 45 | Ac-EGSAFC(1)IWQDWGEHRC(1)[Sar]E-NH2 (SEQ ID NO: 167) |
| 46 | Ac-ESSAIC(1)IWQDWGAHRC(1)T-NH2 (SEQ ID NO: 168) |
| 47 | Ac-IC(1)IWQDWGAHRC(1)TGAES-NH2 (SEQ ID NO: 169) |
| 48 | H-{d}YIC(1)I[1-Me-Trp]QDW[Sar]AHRC(1)[N-Me-Ile]-NH2 |
| | (SEQ ID NO: 170) |
| 49 | Ac-EGSAIC(1)I[1-Me-Trp]QDWGEHRC(1)[Sar]E-NH2 |
| | (SEQ ID NO: 171) |
| 50 | Ac-EGSAIC(1)I[2-Nal]QDWGEHRC(1)[Sar]E-NH2 (SEQ ID NO: 172) |
| 51 | Ac-IC(1)I[1-Me-Trp]QDWGEHRC(1)TGAES-NH2 (SEQ ID NO: 173) |
| 52 | Ac-IC(1)I[2-Nal]QDWGEHRC(1)TGAES-NH2 (SEQ ID NO: 174) |
| 53 | Ac-EGSAFC(1)I[1-Me-Trp]QDWGEHRC(1)[Sar]E-NH2 |
| | (SEQ ID NO: 175) |
| 54 | Ac-EGSAYC(1)I[1-Me-Trp]QDWGEHRC(1)[Sar]E-NH2 |
| | (SEQ ID NO: 176) |
| 55 | Ac-EGSAIC(1)IWQDWGEHRC(1)TE-NH2 (SEQ ID NO: 177) |
| 56 | Ac-EGSAFC(1)I[1-Nal]QDWGEHRC(1)TE-NH2 (SEQ ID NO: 178) |
| 57 | Ac-EGSAFC(1)I[1-Me-Trp]QDWGEHRC(1)TE-NH2 (SEQ ID NO: 179) |
| 58 | Ac-EGSAFC(1)I[1-Me-Trp]QDWGEHRC(1)EGE-NH2 (SEQ ID NO: 180) |
| 59 | Ac-EGSAYC(1)I[1-Me-Trp]QDWGEHRC(1)TE-NH2 (SEQ ID NO: 181) |
| 60 | Ac-EGSAFC(1)I[2-Nal]QDWGEHRC(1)TE-NH2 (SEQ ID NO: 182) |
| 61 | Ac-FC(1)I[1-Me-Trp]QDWGEHRC(1)TGAES-NH2 (SEQ ID NO: 183) |
| 62 | Ac-YC(1)I[1-Me-Trp]QDWGEHRC(1)TGAES-NH2 (SEQ ID NO: 184) |
| 63 | Ac-FC(1)I[1-Nal]QDWGEHRC(1)TGAES-NH2 (SEQ ID NO: 185) |
| 64 | Ac-FC(1)I[2-Nal]QDWGEHRC(1)TGAES-NH2 (SEQ ID NO: 186) |
| 65 | Ac-YC(1)I[2-Nal]QDWGEHRC(1)TGAES-NH2 (SEQ ID NO: 187) |
| 66 | Ac-YC(1)IWQDWGEHRC(1)TGAES-NH2 (SEQ ID NO: 188) |
| 67 | Ac-SEFC(1)I[1-Me-Trp]QDWGEHRC(1)TGAES-NH2 (SEQ ID NO: 189) |
| 68 | Ac-YC(1)I[1-Me-Trp]QDWGEHRC(1)TEAGS-NH2 (SEQ ID NO: 190) |
| 69 | Ac-YC(1)I[1-Me-Trp]QDWGEHRC(1)TESGA-NH2 (SEQ ID NO: 191) |
| 70 | Ac-EGSAYC(1)I[1-Me-Trp]QEWGEHRC(1)[Sar]E-NH2 (SEQ ID NO: 192) |
| 71 | Ac-SEYC(1)I[1-Me-Trp]QDWGEHRC(1)[Sar]EA-NH2 (SEQ ID NO: 193) |
| 72 | Ac-FC(1)I[1-Me-Trp]QDW[Sar]EHRC(1)TGAES-NH2 (SEQ ID NO: 194) |
| 73 | H-{d}YFC(1)I[1-Me-Trp]QDW[Sar]EHRC(1)TGAES-NH2 (SEQ ID NO: 195) |
| 74 | Ac-SEFC(1)I[1-Me-Trp]QDWGEHRC(1)[Sar]GAES-NH2 (SEQ ID NO: 196) |
| 75 | Ac-SEFC(1)I[1-Me-Trp]QDWGEHRC(1)[Sar]EA-NH2 (SEQ ID NO: 197) |
| 76 | Ac-SEFC(1)I[1-Me-Trp]QDW[Sar]EHRC(1)[Sar]EA-NH2 (SEQ ID NO: 198) |
| 77 | Ac-SEFC(1)I[1-Me-Trp]QDW[Sar]EHRC(1)TEA-NH2 (SEQ ID NO: 199) |
| 78 | Ac-SEFC(1)I[1-Me-Trp]QDWGEHRC(1)[Sar]E-NH2 (SEQ ID NO: 200) |
| 79 | Ac-SEFC(1)I[1-Me-Trp]QDW[Sar]EHRC(1)[Sar]E-NH2 (SEQ ID NO: 201) |
| 80 | Ac-EFC(1)I[1-Me-Trp]QDWGEHRC(1)[Sar]EA-NH2 (SEQ ID NO: 202) |
| 81 | Ac-SE[Sar]C(1)I[1-Me-Trp]QDWGEHRC(1)[Sar]EA-NH2 (SEQ ID NO: 203) |
| 82 | Ac-SE[Sar]C(1)I[1-Me-Trp]QDWGEHRC(1)TEA-NH2 (SEQ ID NO: 204) |
| 83 | Ac-SEFC(1)I[1-Me-Trp]QEWGEHRC(1)[Sar]EA-NH2 (SEQ ID NO: 205) |
| 84 | Ac-SEFC(1)I[1-Me-Trp]QDWGEHRC(1)SEA-NH2 (SEQ ID NO: 206) |
| 85 | Ac-EFC(1)I[1-Me-Trp]QDWGEHRC(1)ES-NH2 (SEQ ID NO: 207) |
| 86 | Ac-SEFC(1)I[1-Me-Trp]QDWGEHKC(1)[Sar]EA-NH2 (SEQ ID NO: 208) |
| 87 | Ac-GEFC(1)I[1-Me-Trp]QDWGEHRC(1)[Sar]EA-NH2 (SEQ ID NO: 209) |
| 88 | Ac-GE[Sar]C(1)I[1-Me-Trp]QDWGEHRC(1)TEA-NH2 (SEQ ID NO: 210) |
| 89 | Ac-SE[Sar]C(1)I[1-Me-Trp]QEW[Sar]EHRC(1)TEA-NH2 (SEQ ID NO: 211) |
| 90 | Ac-SE[Sar]C(1)I[1-Me-Trp]QEWGEHRC(1)[Sar]EA-NH2 (SEQ ID NO: 212) |
| 91 | H-{d}Y[Sar]C(1)I[1-Me-Trp]QDWGEHRC(1)TEA-NH2 (SEQ ID NO: 213) |
| 92 | Ac-IC(1)IWQDWGEHRC(1)TEG-K([15-carboxy- |
| | pentadecanoyl][γGlu])-NH2 (SEQ ID NO: 278) |
| 93 | Ac-IC(1)IWQDWGEHRC(1)TEG-K([15-carboxy- |
| | pentadecanoyl][γGlu][Peg3][Peg3])-NH2 (SEQ ID NO: 279) |
| 94 | Ac-IC(1)IWQDWGEHRC(1)TEGE-K([15-carboxy- |
| | pentadecanoyl][γGlu][Peg3][Peg3])-NH2 (SEQ ID NO: 283) |
| 95 | Ac-IC(1)IWQDWGEHRC(1)TEG-K((15-carboxy- |
| | pentadecanoyl)-[(Piperazine-1-yl)-acetyl] |
| | [Peg3][Peg3])-NH2 (SEQ ID NO: 280) |
| 96 | Ac-IC(1)IWQDWGEHRC(1)TEG-K([17-carboxy- |
| | heptadecanoyl][γGlu][Peg3][Peg3])-NH2 (SEQ ID NO: 281) |
| 97 | Ac-IC(1)IWQDWGEHRC(1)TEGE-K([17-carboxy- |
| | heptadecanoyl][γGlu][Peg3][Peg3])-NH2 (SEQ ID NO: 284) |
| 98 | Ac-IC(1)IWQDWGEHRC(1)TEG-K([19-carboxy- |
| | nonadecanoyl][γGlu][Peg3][Peg3])-NH2 (SEQ ID NO: 282) |
| 99 | [15-Carboxy-pentadecanoyl]- |
| | ESSAIC(1)IWQDWGEHRC(1)TEGE-NH2 (SEQ ID NO: 318) |
| 100 | Ac-[K([15-carboxy-pentadecanoyl][γGlu][Peg3]- |
| | [Peg3]GSAIC(1)IWQDWGEHRC(1)TEGE-NH2 (SEQ ID NO: 268) |
| 101 | Ac-EGSAIC(1)IWQDWGEHRC(1)TEG-K([15-carboxy- |
| | pentadecanoyl][γGlu])-NH2 (SEQ ID NO: 271) |
| 102 | Ac-FC(1)I[1-Me-Trp]QDWGEHRC(1)TGAES-K([15-carboxy- |
| | pentadecanoyl][γGlu][Peg3][Peg3])-NH2 (SEQ ID NO: 277) |
| 103 | Ac-EGSAYC(1)I[1-Me-Trp]QDWGEH-[K([15-carboxy- |
| | pentadecanoyl][γGlu][Peg3][Peg3])]-C(1)[Sar]E-NH2 |
| | (SEQ ID NO: 272) |
| 104 | Ac-EGSAYC(1)I[1-Me-Trp]QDWGEHRC(1)[Sar]EG-K([15- |
| | carboxy-pentadecanoyl][γGlu][Peg3][Peg3])-NH2 (SEQ ID NO: 273) |
| 105 | Ac-SAYC(1)I[1-Me-Trp]QDWGEHRC(1)[Sar]E-K([17- |
| | carboxy-heptadecanoyl][γGlu]KG[γGlu])-NH2 (SEQ ID NO: 285) |
| 106 | Ac-SAYC(1)I[1-Me-Trp]QDWGEHRC(1)[Sar]EK([17- |
| | carboxy-heptadecanoyl][γGlu]G[γGlu])-NH2 (SEQ ID NO: 286) |
| 107 | [15-Carboxy-pentadecanoyl]-EGSEYC(1)I[1-Me- |
| | Trp]QDWGEHRC(1)[Sar]E-NH2 (SEQ ID NO: 316) |
| 108 | [17-Carboxy-heptadecanoyl]-EGSEYC(1)I[1-Me- |
| | Trp]QDWGEHRC(1)[Sar]E-NH2 (SEQ ID NO: 317) |
| 109 | Ac-EGSAYC(1)I[1-Me-Trp]QDWGEHRC(1)[Sar]EGE-K([17- |
| | carboxy-heptadecanoyl][γGlu]G[γGlu])-NH2 (SEQ ID NO: 274) |
| 110 | Ac-EGSAYC(1)I[1-Me-Trp]QDWGEHRC(1)[Sar]EGK-K([17- |
| | carboxy-heptadecanoyl][γGlu]G[γGlu])-NH2 (SEQ ID NO: 275) |
| 111 | Ac-EGSAYC(1)I[1-Me-Trp]QDWGEHRC(1)[Sar]EK[γGlu]- |
| | K([17-carboxy-heptadecanoyl][γGlu][Peg3][Peg3])]-NH2 |
| | (SEQ ID NO: 276) |
| 112 | Ac-SEYC(1)I[1-Me-Trp]QDWGEHRC(1)[Sar]EGA-K([17- |
| | carboxy-heptadecanoyl][γGlu]G[γGlu])-NH2 (SEQ ID NO: 307) |
| 113 | Ac-ASGEYC(1)I[1-Me-Trp]QDWGEHRC(1)[Sar]EGE-K([17- |
| | carboxy-heptadecanoyl][γGlu]G[γGlu])-NH2 (SEQ ID NO: 269) |
| 114 | Ac-SEYC(1)I[1-Me-Trp]QDWGEHRC(1)[Sar]EGE-K([17- |
| | carboxy-heptadecanoyl][γGlu]G[γGlu])-NH2 (SEQ ID NO: 310) |
| 115 | Ac-SEYC(1)I[1-Me-Trp]QDWGEHRC(1)[Sar]EGK-K[17- |
| | carboxy-heptadecanoyl][γGlu]G[γGlu])]-NH2 (SEQ ID NO: 311) |
| 116 | Ac-SEYC(1)I[1-Me-Trp]QDWGEHRC(1)[Sar]EGE-K([17- |
| | carboxy-heptadecanoyl][γGlu]K[γGlu])-NH2 (SEQ ID NO: 312) |
| 117 | Ac-SEYC(1)I[1-Me- |
| | Trp]QDWGEHRC(1)[Sar]EGE[Peg3][Peg3]-K([17-carboxy- |
| | heptadecanoyl][γGlu]G[γGlu])-NH2 (SEQ ID NO: 309) |
| 118 | Ac-SEYC(1)I[1-Me-Trp]QDWGEHRC(1)[Sar]EGA-K([17- |
| | carboxy-heptadecanoyl][γGlu]G[Peg3][γGlu][Peg3])-NH2 |
| | (SEQ ID NO: 308) |
| 119 | Ac-SEFC(1)I[1-Me-Trp]QDWGEHRC(1)[Sar]EGA-K([17- |
| | carboxy-heptadecanoyl][γGlu]G[Peg3][γGlu][Peg3])-NH2 |
| | (SEQ ID NO: 287) |
| 120 | Ac-SEFC(1)I[1-Me-Trp]QEWGEHRC(1)[Sar]EGA-K([17- |
| | carboxy-heptadecanoyl][γGlu]G[Peg3][γGlu][Peg3])-NH2 |
| | (SEQ ID NO: 305) |
| 121 | Ac-SEYC(1)I[1-Me-Trp]QEW[Sar]EHRC(1)[Sar]EK[γGlu]A- |
| | K([17-carboxy-heptadecanoyl][γGlu]G[Peg3][γGlu][Peg3])- |
| | NH2 (SEQ ID NO: 313) |
| 122 | Ac-SEYC(1)I[1-Me-Trp]QEWGEHRC(1)[Sar]EGA-K([17- |
| | carboxy-heptadecanoyl][γGlu]G[Peg3][γGlu][Peg3])-NH2 |
| | (SEQ ID NO: 314) |
| 123 | Ac-SEFC(1)I[1-Me-Trp]QDWGEHRC(1)[Sar]EGE- |
| | [Peg3][Peg3]-K([17-carboxy-heptadecanoyl][γGlu]G[γGlu])- |
| | NH2 (SEQ ID NO: 288) |
| 124 | Ac-SEFC(1)I[1-Me-Trp]QEWGEHRC(1)[Sar]EGE- |
| | [Peg3][Peg3]-K([17-carboxy-heptadecanoyl][γGlu]- |
| | G[γGlu])]-NH2 (SEQ ID NO: 306) |
| 125 | Ac-SEYC(1)I[1-Me- |
| | Trp]QEWGEHRC(1)[Sar]EGE[Peg3][Peg3]-K([17-carboxy- |
| | heptadecanoyl][γGlu]G[γGlu])-NH2 (SEQ ID NO: 315) |
| 126 | Ac-SEFC(1)I[1-Me-Trp]QDWGEHRC(1)TEGE[Peg3][Peg3]- |
| | K([17-carboxy-heptadecanoyl][γGlu]G[γGlu])-NH2 |
| | (SEQ ID NO: 302) |
| 127 | Ac-SEFC(1)I[1-Me-Trp]QDWGEHRC(1)- |
| | [Sar]EGE[Peg3][Peg3]-K([15-carboxy-pentadecanoyl][γGlu]- |
| | G[γGlu])-NH2 (SEQ ID NO: 292) |
| 128 | Ac-SEFC(1)I[1-Me-Trp]QDWGEHRC(1)[Sar]EGE- |
| | [Peg3][Peg3]-K([19-carboxy-nonadecanoyl][γGlu]G[γGlu])- |
| | NH2 (SEQ ID NO: 293) |
| 129 | Ac-SEFC(1)I[1-Me-Trp]QDWGEHRC(1)[Sar]EGEGGG- |
| | K([17-carboxy-heptadecanoyl][γGlu]G[γGlu])-NH2 |
| | (SEQ ID NO: 289) |
| 130 | Ac-SEFC(1)I[1-Me-Trp]QDWGEHRC(1)TEGEGGG-K([17- |
| | carboxy-heptadecanoyl][γGlu]G[γGlu])-NH2 (SEQ ID NO: 299) |
| 131 | Ac-SEFC(1)I[1-Me-Trp]QDWGEHRC(1)TEGEGGG-K([15- |
| | carboxy-pentadecanoyl][γGlu]G[γGlu])-NH2 (SEQ ID NO: 300) |
| 132 | Ac-SEFC(1)I[1-Me-Trp]QDWGEHRC(1)[Sar]EK[γGlu]GGG- |
| | K([17-carboxy-heptadecanoyl][γGlu]G[γGlu])-NH2 |
| | (SEQ ID NO: 296) |
| 133 | Ac-SEFC(1)I[1-Me-Trp]QDWGEHRC(1)TEK[γGlu]GGG- |
| | K([17-carboxy-heptadecanoyl][γGlu]G[γGlu])-NH2 |
| | (SEQ ID NO: 303) |
| 134 | Ac-EFC(1)I[1-Me-Trp]QDWGEHRC(1)EGE-K([17-carboxy- |
| | heptadecanoyl][γGlu]G[γGlu])-NH2 (SEQ ID NO: 270) |
| 135 | Ac-SEFC(1)I[1-Me-Trp]QDWGEHRC(1)TGAES-K([15- |
| | carboxy-hexadecanoyl][γGlu]G[γGlu])-NH2 (SEQ ID NO: 304) |
| 136 | Ac-SEFC(1)I[1-Me-Trp]-QDWGEHRC(1)TEGE-[8- |
| | aminooctanoyl]-K([17-carboxy-heptadecanoyl]- |
| | [γGlu]G[γGlu])-NH2 (SEQ ID NO: 297) |
| 137 | Ac-SEFC(1)I[1-Me-Trp]QDWGEHRC(1)TEGE-[8- |
| | aminooctanoyl]-E-K([17-carboxy-heptadecanoyl]- |
| | [γGlu]G[γGlu])-NH2 (SEQ ID NO: 298) |
| 138 | Ac-SEFC(1)I[1-Me-Trp]QDWGEHRC(1)[Sar]EGE-[Peg3]- |
| | K([17-carboxy-heptadecanoyl][γGlu]G[γGlu])-NH2 (SEQ ID NO: 290) |
| 139 | Ac-SEFC(1)I[1-Me-Trp]QDWGEHRC(1)[Sar]EGESES- |
| | K([17-carboxy-heptadecanoyl][γGlu]G[γGlu]])-NH2 (SEQ ID NO: 294) |
| 140 | Ac-SEFC(1)I[1-Me-Trp]QDWGEHRC(1)[Sar]EGE[Peg3]ES- |
| | K([17-carboxy-heptadecanoyl][γGlu]G[γGlu])-NH2 (SEQ ID NO: 291) |
| 141 | Ac-SEFC(1)I[1-Me-Trp]QDWGEHRC(1)[Sar]EGESES- |
| | K([17-carboxy-heptadecanoyl][γGlu])-NH2 (SEQ ID NO: 295) |
| 142 | Ac-SEFC(1)I[1-Me-Trp]QDWGEHRC(1)TEGE[Peg3]ES- |
| | K([17-carboxy-heptadecanoyl][γGlu])-NH2 (SEQ ID NO: 301) |
| 143 | Ac-SEFC(1)I[1-Me-Trp] |
| | QDWGEHR[C(1)[Sar]EGE[Peg3][Peg3][Peg3]-K([17- |
| | carboxy-heptadecanoyl][γGlu]G[γGlu])-NH2 (SEQ ID NO: 319) |
| 144 | Ac-SEFC(1)I[1-Me- |
| | Trp]QDW[Sar]EHRC(1)[Sar]E[Peg3][Peg3]-K([17-carboxy- |
| | heptadecanoyl][γGlu]G[γGlu])-NH2 (SEQ ID NO: 320) |
| 145 | Ac-EF[C(1)I[1-Me-Trp]QDWGEHRC(1)[Sar]EA- |
| | [Peg3][Peg3]-K([17-carboxy-heptadecanoyl][γGlu]G[γGlu])- |
| | NH2 (SEQ ID NO: 321) |

*4W9A—described by Mallik et al., J. Med. Chem. 2005, 48, 274-286 (“V4W/H9A”).
Cp40—decribed by Qu et al., Immunobiology 2013, 281(4): 496-505 (also referred to in that paper as “peptide 14).

### EXAMPLE 2: IN VITRO HAEMOLYSIS ASSAY

### Method

The in vitro effect of the compounds of the present invention was assessed by measuring their inhibitory effect of the classical complement pathway in a haemolysis assay.

Briefly, compounds of the present invention and reference compounds were dissolved in DMSO and diluted in Tris/Casein Assay Buffer (10 mM Tris, 145 mM NaCl, 0.5 mM MgCl₂, 0.15 mM CaCl₂, and 0.1% W/V Casein, adjusted to pH 7.4) as 9-point serial dilutions in a 96 well plate. Sensitized sheep red blood cells (RBC) coated with rabbit anti-sheep erythrocyte antiserum (Complement Technology, Inc., TX, USA) were washed in Tris/Casein Assay Buffer. 50 μL from each well of diluted compound was added to a 96-well plate containing 50 μL diluted human serum (Complement Technology, Inc., TX, USA) and incubated for 15 minutes at room temperature. The serum dilution factor was optimized for every serum batch to obtain 70-90% of maximal haemolysis using the protocol. Then 50 μL sensitized sheep red blood cells were added to all wells (10⁷ per well).

After 30 minutes of incubation at 37° C. with gentle agitation, the reaction was stopped by addition of 50 μL Tris STOP Buffer per well (10 mM EDTA, 10 mM Tris, 145 mM NaCl adjusted to pH 7.4). The RBCs were then removed by centrifugation and the resulting supernatant measured for hemolysis by absorbance at 405 nm.

The response was normalized relative to a positive and negative control (vehicle) to calculate the IC50 from the concentration response curve using the 4-parameter logistic (4PL) nonlinear model for curve fitting. All values are based on n=>2 independent determinations.

TABLE 2
Effect of exchange from valine to isoleucine. Compound 1 differs from the prior
art compound 4W9A only by the presence of Ile instead of Val at position 3.

| | CP | | | | | | | | | | | | | | | |
|---|---|---|---|---|---|---|---|---|---|---|---|---|---|---|---|---|
| | hemolysis | | | | | | | | | | | | | | | |
| Comp | IC50 | | | | | | | | | | | | | | | |
| no | (nM) | | 1 | 2 | 3 | 4 | 5 | 6 | 7 | 8 | 9 | 10 | 11 | 12 | 13 | |
| 1 | 150 | Ac | I | C(1) | I | W | Q | D | W | G | A | H | R | C(1) | T | NH₂ |
| 4W9A | 250 | | | | V | | | | | | | | | | | |

Further compounds were tested as shown below.

TABLE 3
in vitro analysis of inhibition of hemolysis

| Compound | IC50[nM] |
|---|---|
| Compstatin | >5 μM |
| Ac-compstatin | >5 μM |
| 4W9A | <500 |
| Cp40 | <100 |
| 1 | <250 |
| 2 | <100 |
| 3 | <100 |
| 4 | <100 |
| 5 | <250 |
| 6 | <250 |
| 7 | <1000 |
| 8 | <500 |
| 9 | <100 |
| 10 | <100 |
| 11 | <100 |
| 12 | <100 |
| 13 | <100 |
| 14 | <100 |
| 15 | <100 |
| 16 | <100 |
| 17 | <100 |
| 18 | <100 |
| 19 | <250 |
| 20 | <100 |
| 21 | <100 |
| 22 | <100 |
| 23 | <100 |
| 24 | <100 |
| 25 | <100 |
| 26 | <100 |
| 27 | <100 |
| 28 | <100 |
| 29 | <100 |
| 30 | <100 |
| 31 | <100 |
| 32 | <100 |
| 33 | <100 |
| 34 | <250 |
| 35 | <500 |
| 36 | <250 |
| 37 | <250 |
| 38 | <250 |
| 39 | <100 |
| 40 | <250 |
| 41 | <250 |
| 42 | <250 |
| 43 | <100 |
| 44 | <250 |
| 45 | <100 |
| 46 | <100 |
| 47 | <100 |
| 48 | <100 |
| 49 | <100 |
| 50 | <100 |
| 51 | <100 |
| 52 | <100 |
| 53 | <100 |
| 54 | <100 |
| 55 | <250 |
| 56 | <100 |
| 57 | <100 |
| 58 | <100 |
| 59 | <100 |
| 60 | <100 |
| 61 | <100 |
| 62 | <100 |
| 63 | <100 |
| 64 | <100 |
| 65 | <100 |
| 66 | <100 |
| 67 | <100 |
| 68 | <100 |
| 69 | <100 |
| 70 | <100 |
| 71 | <100 |
| 72 | <100 |
| 73 | <100 |
| 74 | <100 |
| 75 | <100 |
| 76 | <100 |
| 77 | <100 |
| 78 | <100 |
| 79 | <100 |
| 80 | <100 |
| 81 | <100 |
| 82 | <100 |
| 83 | <100 |
| 84 | <100 |
| 85 | <100 |
| 86 | <100 |
| 87 | <100 |
| 88 | <100 |
| 89 | <100 |
| 90 | <250 |
| 91 | <100 |
| 92 | <1000 |
| 93 | <500 |
| 94 | <500 |
| 95 | <500 |
| 96 | <1000 |
| 97 | <250 |
| 98 | <500 |
| 99 | <250 |
| 100 | <500 |
| 101 | <500 |
| 102 | <100 |
| 103 | <100 |
| 104 | <100 |
| 105 | <100 |
| 106 | <250 |
| 107 | <100 |
| 108 | <500 |
| 109 | <250 |
| 110 | <250 |
| 111 | <100 |
| 112 | <500 |
| 113 | <500 |
| 114 | <500 |
| 115 | <250 |
| 116 | <500 |
| 117 | <250 |
| 118 | <100 |
| 119 | <100 |
| 120 | <250 |
| 121 | <250 |
| 122 | <500 |
| 123 | <100 |
| 124 | <100 |
| 125 | <500 |
| 126 | <100 |
| 127 | <100 |
| 128 | <100 |
| 129 | <100 |
| 130 | <100 |
| 131 | <100 |
| 132 | <100 |
| 133 | <100 |
| 134 | <100 |
| 135 | <100 |
| 136 | <100 |
| 137 | <100 |
| 138 | <100 |
| 139 | <100 |
| 140 | <100 |
| 141 | <100 |
| 142 | <100 |
| 143 | <100 |
| 144 | <100 |
| 145 | <100 |

The following pairs of compounds, each of which differ only at position 3, show that the effects of replacing valine by isoleucine are seen in compounds having a variety of peptide backbone sequences.

TABLE 4
Direct comparison of valine 3 to isoleucine 3
in combination with modification at
position 9, 11 and/or 13.

| | CP | | | | | | | | | | | | | | | | | | |
|---|---|---|---|---|---|---|---|---|---|---|---|---|---|---|---|---|---|---|---|
| | hemolysis | | | | | | | | | | | | | | | | | | |
| | IC50 | | | | | | | | | | | | | | | | | | |
| Compound | (nM) | | | 1 | 2 | 3 | 4 | 5 | 6 | 7 | 8 | 9 | 10 | 11 | 12 | 13 | | | |
| 2 | 94 | Ac | | I | C(1) | I | W | Q | D | W | G | E | H | R | C(1) | T | | NH₂ | (SEQ ID NO: 41) |
| A | 350 | | | | | V | | | | | | | | | | | | | (SEQ ID NO: 257) |
| 6 | 140 | Ac | | I | C(1) | I | W | Q | D | W | G | S | H | R | C(1) | T | | NH₂ | (SEQ ID NO: 45) |
| B | 360 | | | | | V | | | | | | | | | | | | | (SEQ ID NO: 258) |
| 3 | 69 | Ac | ESSA | I | C(1) | I | W | Q | D | W | G | E | H | R | C(1) | T | | NH₂ | (SEQ ID NO: 140) |
| C | 300 | | | | | V | | | | | | | | | | | | | (SEQ ID NO: 259) |
| 15 | 47 | Ac | | I | C(1) | I | W | Q | D | W | G | E | H | R | C(1) | T | GAES | NH₂ | (SEQ ID NO: 145) |
| D | 210 | | | | | V | | | | | | | | | | | | | (SEQ ID NO: 260) |
| 19 | 140 | Ac | | I | C(1) | I | W | Q | D | W | G | A | H | S | C(1) | T | | NH₂ | (SEQ ID NO: 36) |
| E | >1000 | | | | | V | | | | | | | | | | | | | (SEQ ID NO: 261) |
| 20 | 59 | Ac | | I | C(1) | I | W | Q | D | W | G | E | H | S | C(1) | T | | NH₂ | (SEQ ID NO: 42) |
| F | 540 | | | | | V | | | | | | | | | | | | | (SEQ ID NO: 262) |
| 21 | 77 | Ac | | I | C(1) | I | W | Q | D | W | G | E | H | R | C(1) | S | | NH₂ | (SEQ ID NO: 40) |
| G | 180 | | | | | V | | | | | | | | | | | | | (SEQ ID NO: 263) |
| 28 | 88 | Ac | EGSA | I | C(1) | I | W | Q | D | W | G | E | H | R | C(1) | Sar | E | NH₂ | (SEQ ID NO: 150) |
| H | 330 | | | | | V | | | | | | | | | | | | | (SEQ ID NO: 264) |
| 24 | 90 | Ac | | I | C(1) | I | W | Q | D | W | G | E | H | R | C(1) | T | EGE | NH₂ | (SEQ ID NO: 146) |
| J | 240 | | | | | V | | | | | | | | | | | | | (SEQ ID NO: 265) |

Isoleucine at position 3 was also demonstrated to be superior compared to other residues often considered to be “conservative” replacements for isoleucine.

TABLE 5
Effect on hemolysis of different
residues at position 3
Ac-IC(1)XWQDWGEHRC(1)T-NH2

| | | IC50, CP |
|---|---|---|
| | | hemolysis |
| Compound | Position 3 (X) | (nM) |
| A | Valine | 350 |
| 2 | Isoleucine | <100 |
| — | Leucine | 500 |
| — | Norvaline | >1000 |
| — | Norleucine | 480 |
| — | Phenylalanine | >10000 |
| — | Beta-Homo- | >10000 |
| | Isoleucine | |

Due to the high concentration of C3 found in serum, it may be difficult to use the hemolysis assay to differentiate between compounds having very high affinity for C3.

In such circumstances, it may be possible to determine a more accurate hierarchy of binding affinity to C3 by SPR measurements using immobilized C3, as described below.

### EXAMPLE 3: SOLUBILITY TEST

### Materials and Method

Compound solubility at 10 mg/ml

The solubility of compounds was assessed by measuring light scattering over a pH interval from pH 4 to pH 7.5.

Compounds were dissolved in a stock solution of 20 mg/mL in H₂O at pH 2.5 or pH 10. These stock solutions were diluted 1:1 with 200 mM buffered solution to reach a final solution of 10 mg/mL compound in 100 mM buffer. The 5 investigated conditions were (1) acetate pH 4.0, (2) acetate pH 5.0, (3) phosphate pH 6.0, (4) phosphate pH 7 and (5) phosphate pH 7.5.

These samples were equilibrated for 15 minutes at ambient temperature, before evaluating solubility by visual inspection and absorbance measurements in a SpectraMax 190 microplate reader (Molecular Devices).

### Visual Inspection

Visual inspection included manually checking the 96 well plate for wells that are clear or non-clear. In addition to this a picture of the 96 well plate is taken.

### Microplate Reader and Light Scattering

Absorbance was measured at four wavelengths: 280 nm, 325 nm, 340 nm and 360 nm in an UV transparent 96 well microplate in a SpectraMax 190 microplate reader (Molecular Devices). The compounds do not absorb at 325-360 nm and signal at these wavelengths are therefore an expression of light scattering, which reflects the presence of visible or sub-visible particles that are detected as increased signal.

The light scattering was normalized to the signal from pure buffer solutions (100 mM) and compound solubility was evaluated as good (+) or poor (−). The criteria for this was a combination of visual inspection and light scattering not exceeding 0.1 AU, where values below 0.1 AU are good in visually clear samples.

### Solubility of Comp No 24

### Stock Solution

Comp No 24 was carefully weighed out and dissolved in pH 2.5 H₂O—Cl. The stock solution was equilibrated 15 minutes at ambient temperature, at which point no visible particles were present. 200 mM buffer stock solutions were prepared for each pH condition.

### Solubility Assay

The formulations for solubility testing were made by mixing 50 μL Comp No 24 stock solution and 50 μL buffer stock solution with gentle mixing by pipetting the solution a couple of times. This was done for each buffer/pH condition in a UV transparent 96 well microplate (Corning 96 well REF 3635). Reference samples without Comp No 24 were made by mixing 50 μL pH 2.5 H₂O—Cl and 50 μL buffer stock solution. The plate was covered with a lid and left 15 minutes at ambient temperature before assessing solubility.

### Measuring Solubility

Solubility was assessed by visual inspection of each formulation and a picture taken in a photo box. Light scattering was measured at 280 nm, 325 nm, 340 nm and 360 nm in a SpectraMax 190 microplate reader (Molecular Devices).

The visual inspection revealed that condition 1, 2 and 3 were cloudy and condition 2 additionally contained visible precipitates. The absorbance measurement confirmed the visual evaluation with condition 1, 2 and 3 all exceeding 0.1 AU threshold. Condition 4 and 5 were thus deemed good conditions for solubility of 10 mg/mL Comp No 24.

Similarly, additional compounds were tested for solubility (Table 6).

TABLE 6
Table of most soluble compounds, as tested at 10 mg/mL.

| | Buffer & pH | | | | |
|---|---|---|---|---|---|
| | Condi- | Condi- | Condi- | Condi- | Condi- |
| | tion 1 | tion 2 | tion 3 | tion 4 | tion 5 |
| | Acetate | Acetate | Phosphate | Phosphate | Phosphate |
| Comp No | pH 4 | pH 5 | pH 6 | pH 7 | pH 7.5 |
| 1 | + | − | − | − | − |
| 3 | + | − | − | + | + |
| 14 | + | − | − | + | + |
| 15 | + | − | − | + | + |
| 22 | + | − | − | + | + |
| 24 | − | − | − | + | + |
| 25 | + | − | − | + | + |
| 27 | − | − | − | + | + |
| 28 | + | + | + | + | + |
| 30 | − | − | | + | + |
| 31 | + | + | + | + | + |
| 32 | − | − | − | + | + |
| 33 | − | − | + | + | + |
| 36 | − | − | − | + | + |
| 40 | − | − | + | + | + |
| 41 | − | − | + | + | + |
| 44 | − | − | + | + | + |
| 45 | − | + | + | + | + |
| 49 | − | − | + | + | + |
| 50 | − | − | + | + | + |
| 51 | − | − | + | + | + |
| 52 | − | − | + | + | + |
| 53 | − | − | + | + | + |
| 54 | − | − | + | + | + |
| 55 | − | − | + | + | + |
| 56 | − | − | + | + | + |
| 57 | − | − | + | + | + |
| 60 | − | − | + | + | + |
| 61 | − | − | + | + | + |
| 62 | − | − | + | + | + |
| 63 | − | − | + | + | + |
| 65 | − | − | + | + | + |
| 66 | − | − | + | + | + |
| 67 | − | − | + | + | + |
| 68 | − | − | + | + | + |
| 72 | − | − | + | + | + |
| 73 | + | − | − | + | + |
| 74 | − | + | + | + | + |
| 76 | − | − | + | + | + |
| 77 | − | − | + | + | + |
| 78 | − | − | + | + | + |
| 79 | − | − | + | + | + |
| 80 | − | − | + | + | + |
| 81 | − | − | + | + | + |
| 102 | − | + | + | + | + |
| 103 | − | + | + | + | + |
| 104 | − | + | + | + | + |
| 105 | − | − | + | + | + |
| 107 | − | − | + | + | + |
| 108 | − | − | + | + | + |
| 109 | − | + | + | + | + |
| 111 | − | − | + | + | + |
| 114 | + | + | + | + | + |
| 115 | − | − | + | + | + |
| 116 | − | − | + | + | + |
| 118 | − | + | + | + | + |

“+” denotes solubility at the given condition, as determined by UV absorbance being less than 0.1 AU at 340 nm and the sample being clear when manually inspected.
“−” denotes lack of solubility at the given condition, as UV absorbance at 340 nm exceeds 0.1 AU and/or it is visibly turbid or contains particles.

### EXAMPLE 4: AFFINITY MEASUREMENTS BY SURFACE PLASMON RESONANCE (SPR)

### Method

Surface plasmon resonance (SPR) was used to characterize peptides with respect to their binding affinity (Kd) for C3. Human C3 (Complement tech cat #A113c) was immobilised on individual flow cells of CM5 sensor chips (GE Healthcare) using standard amine coupling to a density of approximately 3000 resonance units (RU) in a buffer consisting of 10 mM phosphate pH 7.4, 150 mM NaCl, 0.05% Tween20.

For interaction experiments a multi-cycle experiment approach was used and performed using a BiacoreT200™ instrument (GE Healthcare) at 25° C. Peptides were injected in increasing concentration series (6-8 different concentrations) for 60-120 s at a flow rate of 30 μL/min in a buffer consisting of 10 mM Tris buffer at pH 7.4, with 150 mM NaCl and 0.05% Tween20. This was followed by a dissociation period for up to 10 min. The C3 surface was regenerated between runs by a 45 s injection of 3 M MgCl₂.

Sensorgrams were double-referenced (reference surface, blanks) prior to analysis of the kinetic profiles by globally fitting data to a 1:1 Langmuir binding model to obtain association and dissociation rates for calculation of the equilibrium dissociation constant Kd. Each peptide was tested at in a least 3 independent experiments.

TABLE 7
Compstatin analogues binding affinities for C3 as determined
by a surface plasmon resonance assay with immobilized C3.

| Comp. no. | Kd [nM] | N |
|---|---|---|
| 2 | 16 | 3 |
| 4 | 1.5 | 3 |
| 15 | 14 | 3 |
| 20 | 37 | 3 |
| 21 | 16 | 3 |
| 23 | 2.8 | 3 |
| 24 | 28 | 5 |
| 28 | 44 | 3 |
| 29 | 21 | 3 |
| 43 | 3.3 | 3 |
| 48 | 0.12 | 3 |
| 49 | 3.2 | 3 |
| 50 | 13 | 3 |
| 53 | 1.4 | 3 |
| 54 | 3.0 | 3 |
| 61 | 0.33 | 3 |
| 63 | 4.3 | 3 |
| 67 | 0.68 | 7 |
| 73 | 0.30 | 3 |
| 75 | 1.5 | 3 |
| 81 | 9.7 | 3 |
| 82 | 5.4 | 3 |
| 85 | 1.3 | 3 |
| 86 | 2.6 | 3 |
| 102 | 1.7 | 3 |
| 104 | 34 | 2 |
| 106 | 5.4 | 5 |
| 107 | 6.1 | 5 |
| 111 | 8.2 | 5 |
| 117 | 24 | 3 |
| 118 | 11 | 5 |
| 119 | 9.8 | 3 |
| 120 | 28 | 3 |
| 121 | 30 | 3 |
| 122 | 63 | 3 |
| 123 | 11 | 3 |
| 124 | 31 | 3 |
| 125 | 71 | 3 |
| 126 | 5.2 | 3 |
| 127 | 8.5 | 3 |
| 128 | 6.5 | 3 |
| 130 | 4.4 | 3 |
| 139 | 7.4 | 3 |
| 140 | 7.6 | 3 |
| 141 | 6.6 | 3 |
| 142 | 4.8 | 3 |

The following pairs of compounds, which differ only at position 3, show the effects of replacing valine by isoleucine in different peptide backbones.

TABLE 8
Binding affinity of compstatin analogues to
immobilized C3 determined by a surface
plasmon resonance (SPR) assay.

| Comp | spr Kd | | | | | | | | | | | | | | | | | | |
|---|---|---|---|---|---|---|---|---|---|---|---|---|---|---|---|---|---|---|---|
| no | (nM) | | | 1 | 2 | 3 | 4 | 5 | 6 | 7 | 8 | 9 | 10 | 11 | 12 | 13 | | | |
| 2 | 16 | Ac | | I | C(1) | I | W | Q | D | W | G | E | H | R | C(1) | T | | NH₂ | (SEQ ID |
| | | | | | | | | | | | | | | | | | | | NO: 41) |
| A | 130 | | | | | V | | | | | | | | | | | | | (SEQ ID |
| | | | | | | | | | | | | | | | | | | | NO: 257) |
| 15 | 14 | Ac | | I | C(1) | I | W | Q | D | W | G | E | H | R | C(1) | T | GAES | NH₂ | (SEQ ID |
| | | | | | | | | | | | | | | | | | | | NO: 145) |
| D | 230 | | | | | V | | | | | | | | | | | | | (SEQ ID |
| | | | | | | | | | | | | | | | | | | | NO: 260) |
| 21 | 16 | Ac | | I | C(1) | I | W | Q | D | W | G | E | H | R | C(1) | S | | NH₂ | (SEQ ID |
| | | | | | | | | | | | | | | | | | | | NO: 40) |
| G | 160 | | | | | V | | | | | | | | | | | | | (SEQ ID |
| | | | | | | | | | | | | | | | | | | | NO: 263) |
| 48 | 0.12 | H | dTyr | I | C(1) | I | 1MeTrp | Q | D | W | Sar | A | H | R | C(1) | NMelle | | NH₂ | (SEQ ID |
| | | | | | | | | | | | | | | | | | | | NO: 170) |
| Cp40 | 0.31 | | | | | V | | | | | | | | | | | | | (SEQ ID |
| | | | | | | | | | | | | | | | | | | | NO: 326) |

### EXAMPLE 5: PROFILING OF TEST COMPOUNDS IN NON-HUMAN PRIMATES (NHP)

Healthy male Cynomolgus monkeys (Macaca fascicularis) received single subcutaneous administrations of each test substance. Compounds were formulated in 20 mM phosphate adjusted with NaOH to pH 7.5 and mannitol for isotonicity and dosed at 1840 nmol/kg. Blood was collected from a femoral vein from each animal at the following times: Pre-dose, 1, 2, 4, 8, 24, 48, 72, 96 and 120 h (10 sampling times). Blood was collected into serum separation tubes and allowed to clot at room temperature. The tubes were centrifuged and resulting serum was aliquoted and snap-frozen over dry-ice and stored at nominally −80° C. until analysis. All NHP studies were performed in accordance with animal welfare laws and regulations, including approval of the study by a local ethical review process.

Serum isolated from non-human primates at specific time points after dosing were analyzed for alternative pathway complement activity using the Complement system Alternative Pathway WIESLAB® kit from Svar Life Science (previously Euro diagnostic AB, Sweden) following the manufacturer's protocol. Briefly, serum samples or controls were diluted in buffer and incubated in microtitre strips coated with specific activators of the alternative pathway. The wells were washed and formed C5b-9 was detected using included colorimetric reagents. Absorbance at 405 nm was measured. The percent activity of the alternative complement pathway was calculated for each animal and timepoint relative to the pre-dose activity (0 hours) of the individual animal with subtraction of the negative control. This reflects the pharmacological activity of the compounds.

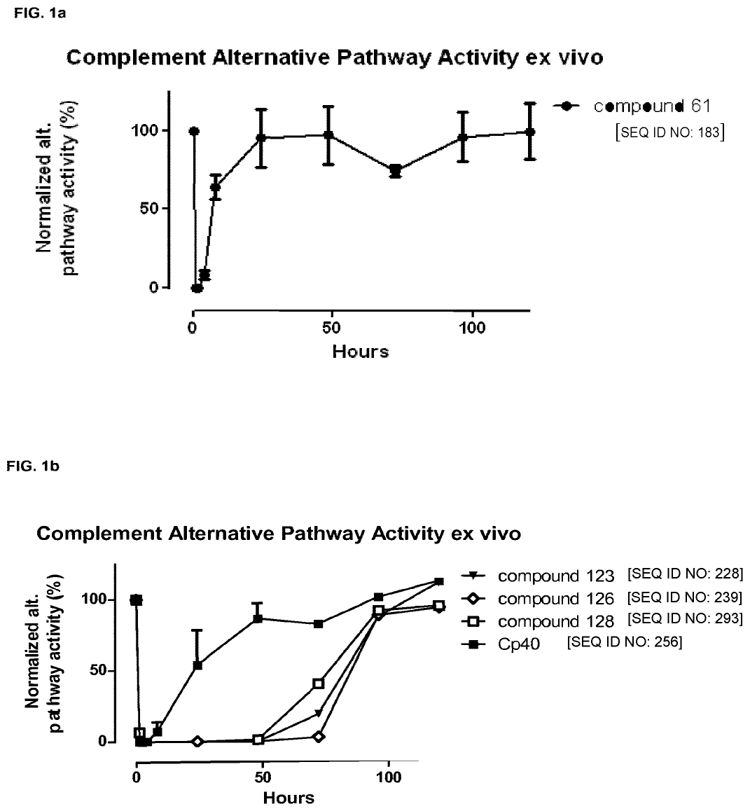

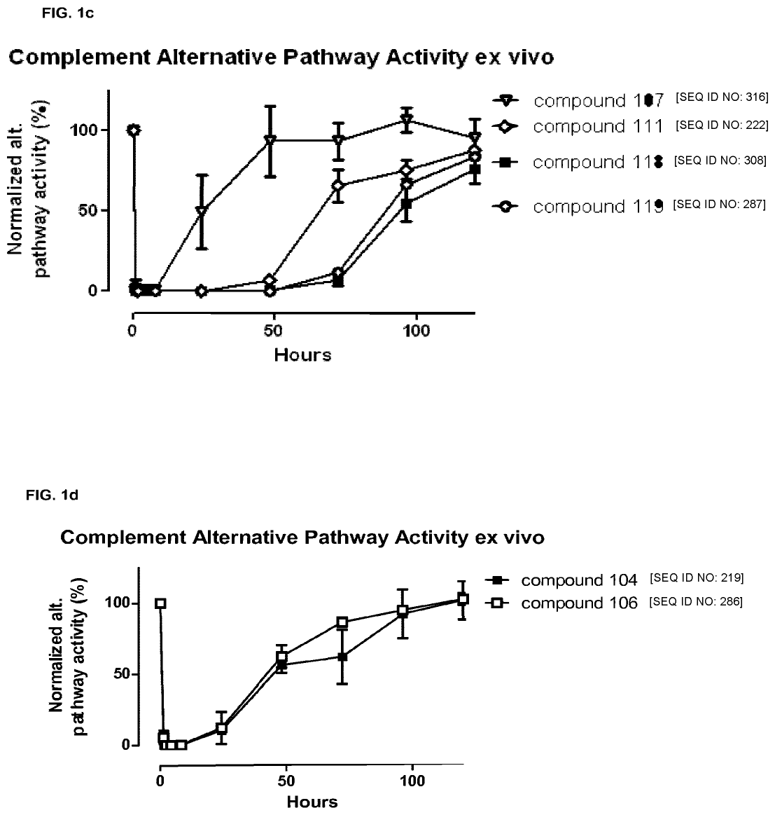

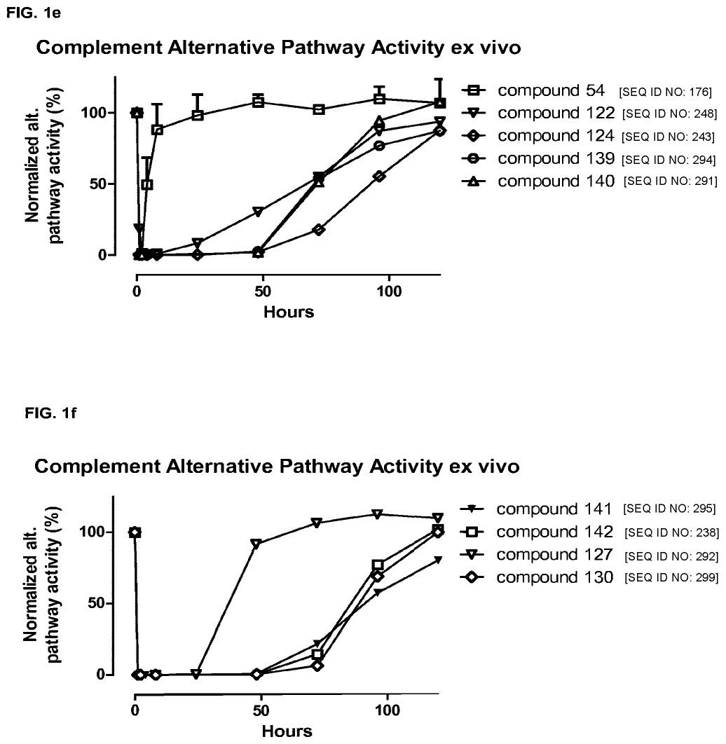

The results from the Alternative Pathway WIESLAB® kit are shown in FIGS. 1a-f. In FIG. 1a, the non-acylated compound 61 had a relatively short duration of action despite high affinity for C3. The same is seen for the non-acylated compounds Cp40 (FIG. 1b) and compound 54 (FIG. 1e). By contrast, the acylated compounds in FIGS. 1b, 1c, 1d, 1e and 1f in general possessed a longer-lasting pharmacological activity in vivo when compared to the non-acylated compounds despite lower affinity. Although acylation of peptides is generally known to increase the in vivo half-life, it was surprisingly found that the in vivo duration of the pharmacological efficacy was prolonged to this extent.

In order to assess pharmacokinetic half-life (t½), serum samples isolated from non-human primates at specific time points after dosing were analysed for total drug compound after sample preparation by solid phase extraction (SPE) and liquid chromatography mass spectrometry (LC-MS/MS) using analogue internal standard. Single measurement of serum concentrations were used for calculation of the pharmacokinetic parameters using the non-compartmental approach in Phoenix WinNonlin 6.3. Plasma terminal elimination half-life (t_{1/2}) was determined as ln(2)/λz where λz is the magnitude of the slope of the log linear regression of the log concentration versus time profile during the terminal phase.

Pharmacokinetic (PK) data are shown in Table 9.

TABLE 9
PK data in NHP:

| | t_{1/2} |
|---|---|
| Compound | hours |
| Cp40 | 31.8 |
| 54 | 9.71 |
| 61 | 23.3 |
| 104 | 96.3* |
| 106 | 93.9* |
| 107 | 20.1 |
| 111 | 157* |
| 118 | 78.7* |
| 118 | 155 |
| 119 | 139 |
| 122 | 127 |
| 123 | 105 |
| 124 | 112 |
| 139 | 82 |
| 140 | 100 |
| 141 | 145 |
| 142 | 143 |

*Approximate determination, as t½ determined over less than three times the expected half-life.

1. A compstatin analogue represented by the formula:
Y1-R1-X1-C-I-X4-Q-X6-W-X8-E-H-X11-C-X13-R2-Y2 (Formula I)
wherein:
Y1 is hydrogen, acetyl or a lipophilic group Φ;
X1 is I, Y, F or Sar;
X4 is W, F, V, Y, 1-Me-Trp, D-Trp, N-Me-Trp, 1-For-Trp, 1-Nal, 2-Nal, 5-Me-Trp, Bpa or 2-Igl;
X6 is E, K or D;
X8 is G or Sar;
X11 is R, S or K;
X13 is T, S, E, F, H, K, Sar, G, I, D, N-Me-Ile or N-Me-Thr;
Y2 is NH₂, OH or a lipophilic group Φ;
R1 is absent or is a sequence of 1 to 6 amino acid residues selected from A, E, G, L, K, F, P, S, T, W, Y, R, V, Sar, ¿Lys, γGlu, βAsp, or βAla, or a corresponding D form thereof; or Peg3, Peg4, or 8-aminooctanoyl, or derivatives thereof; and
R2 is absent or is a sequence of 1 to 8 amino acid residues selected from A, E, G, L, K, F, P, S, T, W, Y, R, V, Sar, ¿Lys, γGlu, βAsp, or βAla, or a corresponding D form thereof; or Peg3, Peg4, or 8-aminooctanoyl, or derivatives thereof;
wherein the compstatin analogue has a disulphide bond between the cysteine residues at positions 2 and 12;
and wherein the compstatin analogue optionally has a lipophilic group Φ covalently linked to the side chain of one or more amino acid residues;
or a pharmaceutically acceptable salt and/or solvate thereof.

2-4. (canceled)

5. A compstatin analogue according to claim 1 comprising at least one lipophilic group Φ, wherein:
(a) Y1 or Y2 is a lipophilic group Φ; and/or
(b) the compstatin analogue comprises a lipophilic group Φ linked to the side chain of an amino acid residue at position X1, X11 or X13, or an amino acid residue in R1 or R2, wherein said amino acid residue is a lysine residue.

6-8. (canceled)

9. A compstatin analogue according to claim 1 which does not comprise a lipophilic group Φ.

10-15. (canceled)

16. A compstatin analogue according to claim 1, wherein the 13-mer peptide portion (X1-X13) of the compstatin analogue has a sequence selected from:
[Sar]C(1)I[1-Me-Trp]QDWGEHRC(1)[Sar];
[Sar]C(1)I[1-Me-Trp]QDWGEHRC(1)T;
[Sar]C(1)I[1-Me-Trp]QEW[Sar]EHRC(1)T;
[Sar]C(1)I[1-Me-Trp]QEWGEHRC(1)[Sar];
[Sar]C(1)IWQDWGEHRC(1)T;
FC(1)I[1-Me-Trp]QDW[Sar]EHRC(1)[Sar];
FC(1)I[1-Me-Trp]QDW[Sar]EHRC(1)T;
FC(1)I[1-Me-Trp]QDWGEHKC(1)[Sar];
FC(1)I[1-Me-Trp]QDWGEHRC(1)[Sar];
FC(1)I[1-Me-Trp]QDWGEHRC(1)E;
FC(1)I[1-Me-Trp]QDWGEHRC(1)S;
FC(1)I[1-Me-Trp]QDWGEHRC(1)T;
FC(1)I[1-Me-Trp]QEWGEHRC(1)[Sar];
FC(1)I[1-Nal]QDWGEHRC(1)T;
FC(1)I[2-Nal]QDWGEHRC(1)T;
FC(1)IWQDWGEHRC(1)[Sar];
FC(1)IWQDWGEHRC(1)T;
IC(1)I[1-Me-Trp]QDWGEHRC(1)[Sar];
IC(1)I[1-Me-Trp]QDWGEHRC(1)T;
IC(1)I[2-Nal]QDWGEHRC(1)[Sar];
IC(1)IWQDWGEHRC(1)[Sar];
IC(1)IWQDWGEHRC(1)E;
IC(1)IWQDWGEHRC(1)S;
IC(1)IWQDWGEHRC(1)T;
IC(1)IWQDWGEHSC(1)T;
IC(1)IWQEWGEHRC(1)T;
IC(1)IWQKWGEHRC(1)T;
YC(1)I[1-Me-Trp]QDWGEHRC(1)[Sar];
YC(1)I[1-Me-Trp]QDWGEHRC(1)T;
YC(1)I[1-Me-Trp]QEWGEHRC(1)[Sar];
YC(1)I[2Nal]QDWGEHRC(1)T;
YC(1)IWQDWGEHRC(1)T;
YC(1)I[1-Me-Trp]QDWGEH[K*]C(1)[Sar]; and
YC(1)I[1-Me-Trp]QEW[Sar]EHRC(1)[Sar].

17. A compstatin analogue according to claim 1, wherein:
(a) R1 has a sequence selected from:
{d}Y, EGSE, AGSE, SASE, EYSE, GSE, ASE, ESSA, KGSA, AKGE, ASGE, ASSE, ASES, GSAE, ESSE, ESGA, SEG, GES, ESS, EGSA, ESE, EGE, ESA, SAE, SGA, YLEA, GSA, KEK, EKG, ES, AE, TE, KE, GE, FE, YE, AS, SE, RS, SR, SA, GE, Y, S and E; and/or
(b) R2 has a sequence selected from:
EGASGSG, EGAGSG, EGASAG, EGAGAG, EGESGSG, EGEGSG, EGESAG, EGEGAG, EK[γGlu]AK, EK[γGlu]A, EGEGG, EGAGG, EGESS, GAESK, EGAK, EGEK, EGG, EGK, EGKK, EGS, EK, EGA, EGAK, EK[γGlu], EK[γGlu]-K, EGE[Peg3], EGE[Peg3]-K, EGE[Peg3][Peg3], EGE[Peg3][Peg3]-K, EGE[Peg3][Peg3][Peg3], EGE[Peg3][Peg3][Peg3]-K GESESE, GAESES, EGESES, EGESESK, EGE[Peg3]-ES, EGE[Peg3]-ESK, GESESE, EGE-[8-aminooctanoyl], EGE-[8-aminooctanoyl]-K, EGE-[8-aminooctanoyl]-EK, EGEGGG, EGEGGGK, EK[γGlu]GGG, EK[γGlu]GGGK, EGE-[8-aminooctanoyl]-E, E[Peg3][Peg3], E[Peg3][Peg3]-K, EA[Peg3][Peg3], EA[Peg3][Peg3]-K, GAES, EYGS, EGYA, EAGS, EAKS, EKSA, ESGA, EGGS, EGGA, ESSG, ESAG, GEES, AEES, ESEG, AEGS, ESGS, SEGA, SEG, EGK, ESG, EAG, GAE, EGEA, EGE, EA, E, S, GE, GEK, EG, EA, EKE or EKP.

18. A compstatin analogue according to claim 17 comprising a lipophilic group Φ covalently linked to an amino acid side chain of R1 and/or R2.

19. A compstatin analogue according to claim 18 wherein:
(a) R1 has the sequence K*GSA; and/or
(b) R2 has the sequence EK[γGlu]AK*, EGKK*, EK[γGlu]K*, EGE[Peg3]-K*, EGESESK*, EGE[Peg3]-ESK*, EGE-[8-aminooctanoyl]-K*, EGE-[8-aminooctanoyl]-EK*, EGEGGGK*, EK[γGlu]GGGK*, EGE[Peg3][Peg3]-K*, EGE[Peg3][Peg3][Peg3]-K*, E[Peg3][Peg3]-K*, EA[Peg3][Peg3]-K*, GAESK*, EGAK*, EGEK*, EGK* EGE[Peg3]-ESK*, GESESEK*, GEK* or EK*.

20-22. (canceled)

23. A compstatin analogue according to claim 1, comprising a sequence selected from:
IC(1)IWQDWGEHRC(1)T
ESSAIC(1)IWQDWGEHRC(1)T
IC(1)I[1MeTrp]QDWGEHRC(1)T
IC(1)IWQKWGEHRC(1)T
YC(1)IWQDWGEHRC(1)T
ESSAYC(1)IWQDWGEHRC(1)T
[Sar]C(1)IWQDWGEHRC(1)T
IC(1)IWQDWGEHRC(1)[Sar]
ESSAIC(1)IWQDWGEHRC(1)TGAES
IC(1)IWQDWGEHRC(1)TGAES
IC(1)IWQEWGEHRC(1)T
IC(1)IWQDWGEHSC(1)T
IC(1)IWQDWGEHRC(1)S
IC(1)IWQDWGEHRC(1)E
FC(1)IWQDWGEHRC(1)T
IC(1)IWQDWGEHRC(1)TEGE
IC(1)IWQDWGEHRC(1)TEA
IC(1)IWQDWGEHRC(1)TE
IC(1)IWQDWGEHRC(1)EGE
EGSAIC(1)IWQDWGEHRC(1)[Sar]E
EGSAIC(1)IWQDWGEHRC(1)T
EGEIC(1)IWQDWGEHRC(1)T
ESEIC(1)IWQDWGEHRC(1)T
SEIC(1)IWQDWGEHRC(1)TEA
EIC(1)IWQDWGEHRC(1)TE
EIC(1)IWQDWGEHRC(1)TEGE
EGEIC(1)IWQDWGEHRC(1)EGE
ESEIC(1)IWQDWGEHRC(1)EGE
KEKIC(1)IWQDWGEHRC(1)TEKE
EKGIC(1)IWQDWGEHRC(1)TEKP
IC(1)IWQDWGEHRC(1)TEGK
GSAIC(1)IWQDWGEHRC(1)[Sar]E
SAIC(1)IWQDWGEHRC(1)[Sar]E
SAIC(1)IWQDWGEHRC(1)TEG
FC(1)IWQDWGEHRC(1)TGAE
EGSAIC(1)IWQDWGEHRC(1)[Sar]EGE
EGSAFC(1)IWQDWGEHRC(1)[Sar]E
EGSAIC(1)I[1-Me-Trp]QDWGEHRC(1)[Sar]E
EGSAIC(1)I[2-Nal]QDWGEHRC(1)[Sar]E
IC(1)I[1-Me-Trp]QDWGEHRC(1)TGAES
IC(1)I[2-Nal]QDWGEHRC(1)TGAES
EGSAFC(1)I[1-Me-Trp]QDWGEHRC(1)[Sar]E
EGSAYC(1)I[1-Me-Trp]QDWGEHRC(1)[Sar]E
EGSAIC(1)IWQDWGEHRC(1)TE
EGSAFC(1)I[1-Nal]QDWGEHRC(1)TE
EGSAFC(1)I[1-Me-Trp]QDWGEHRC(1)TE
EGSAFC(1)I[1-Me-Trp]QDWGEHRC(1)EGE
EGSAYC(1)I[1-Me-Trp]QDWGEHRC(1)TE
EGSAFC(1)I[2-Nal]QDWGEHRC(1)TE
FC(1)I[1-Me-Trp]QDWGEHRC(1)TGAES
YC(1)I[1-Me-Trp]QDWGEHRC(1)TGAES
FC(1)I[1-Nal]QDWGEHRC(1)TGAES
FC(1)I[2-Nal]QDWGEHRC(1)TGAES
YC(1)I[2-Nal]QDWGEHRC(1)TGAES
YC(1)IWQDWGEHRC(1)TGAES
SEFC(1)I[1-Me-Trp]QDWGEHRC(1)TGAES
YC(1)I[1-Me-Trp]QDWGEHRC(1)TEAGS
YC(1)I[1-Me-Trp]QDWGEHRC(1)TESGA
EGSAYC(1)I[1-Me-Trp]QEWGEHRC(1)[Sar]E
SEYC(1)I[1-Me-Trp]QDWGEHRC(1)[Sar]EA
FC(1)I[1-Me-Trp]QDW[Sar]EHRC(1)TGAES
{d}YFC(1)I[1-Me-Trp]QDW[Sar]EHRC(1)TGAES
SEFC(1)I[1-Me-Trp]QDWGEHRC(1)[Sar]GAES
SEFC(1)I[1-Me-Trp]QDWGEHRC(1)[Sar]EA
SEFC(1)I[1-Me-Trp]QDW[Sar]EHRC(1)[Sar]EA
SEFC(1)I[1-Me-Trp]QDW[Sar]EHRC(1)TEA
SEFC(1)I[1-Me-Trp]QDWGEHRC(1)[Sar]E
SEFC(1)I[1-Me-Trp]QDW[Sar]EHRC(1)[Sar]E
EFC(1)I[1-Me-Trp]QDWGEHRC(1)[Sar]EA
SE[Sar]C(1)I[1-Me-Trp]QDWGEHRC(1)[Sar]EA
SE[Sar]C(1)I[1-Me-Trp]QDWGEHRC(1)TEA
SEFC(1)I[1-Me-Trp]QEWGEHRC(1)[Sar]EA
SEFC(1)I[1-Me-Trp]QDWGEHRC(1)SEA
EFC(1)I[1-Me-Trp]QDWGEHRC(1)ES
SEFC(1)I[1-Me-Trp]QDWGEHKC(1)[Sar]EA
GEFC(1)I[1-Me-Trp]QDWGEHRC(1)[Sar]EA
GE[Sar]C(1)I[1-Me-Trp]QDWGEHRC(1)TEA
SE[Sar]C(1)I[1-Me-Trp]QEW[Sar]EHRC(1)TEA
SE[Sar]C(1)I[1-Me-Trp]QEWGEHRC(1)[Sar]EA
{d}Y[Sar]C(1)I[1-Me-Trp]QDWGEHRC(1)TEA

24. A compstatin analogue according to claim 1 which is:
Ac-IC(1)IWQDWGEHRC(1)T-NH2 (Compound 2)
Ac-ESSAIC(1)IWQDWGEHRC(1)T-NH2 (Compound 3)
Ac-IC(1)I[1-Me-Trp]QDWGEHRC(1)T-NH2 (Compound 4)
Ac-IC(1)IWQKWGEHRC(1)T-NH2 (Compound 7)
Ac-YC(1)IWQDWGEHRC(1)T-NH2 (Compound 9)
Ac-ESSAYC(1)IWQDWGEHRC(1)T-NH2 (Compound 10)
Ac-[Sar]C(1)IWQDWGEHRC(1)T-NH2 (Compound 11)
Ac-IC(1)IWQDWGEHRC(1)[Sar]-NH2 (Compound 13)
Ac-ESSAIC(1)IWQDWGEHRC(1)TGAES-NH2 (Compound 14)
Ac-IC(1)IWQDWGEHRC(1)TGAES-NH2 (Compound 15)
Ac-IC(1)IWQEWGEHRC(1)T-NH2 (Compound 16)
Ac-IC(1)IWQDWGEHSC(1)T-NH2 (Compound 20)
Ac-IC(1)IWQDWGEHRC(1)S-NH2 (Compound 21)
Ac-IC(1)IWQDWGEHRC(1)E-NH2 (Compound 22)
Ac-FC(1)IWQDWGEHRC(1)T-NH2 (Compound 23)
Ac-IC(1)IWQDWGEHRC(1)TEGE-NH2 (Compound 24)
Ac-IC(1)IWQDWGEHRC(1)TEA-NH2 (Compound 25)
Ac-IC(1)IWQDWGEHRC(1)TE-NH2 (Compound 26)
Ac-IC(1)IWQDWGEHRC(1)EGE-NH2 (Compound 27)
Ac-EGSAIC(1)IWQDWGEHRC(1)[Sar]E-NH2 (Compound 28)
Ac-EGSAIC(1)IWQDWGEHRC(1)T-NH2 (Compound 29)
Ac-EGEIC(1)IWQDWGEHRC(1)T-NH2 (Compound 30)
Ac-ESEIC(1)IWQDWGEHRC(1)T-NH2 (Compound 31)
Ac-SEIC(1)IWQDWGEHRC(1)TEA-NH2 (Compound 32)
Ac-EIC(1)IWQDWGEHRC(1)TE-NH2 (Compound 33)
Ac-EIC(1)IWQDWGEHRC(1)TEGE-NH2 (Compound 34)
Ac-EGEIC(1)IWQDWGEHRC(1)EGE-NH2 (Compound 35)
Ac-ESEIC(1)IWQDWGEHRC(1)EGE-NH2 (Compound 36)
Ac-KEKIC(1)IWQDWGEHRC(1)TEKE-NH2 (Compound 37)
Ac-EKGIC(1)IWQDWGEHRC(1)TEKP-NH2 (Compound 38)
Ac-IC(1)IWQDWGEHRC(1)TEGK-NH2 (Compound 39)
Ac-GSAIC(1)IWQDWGEHRC(1)[Sar]E-NH2 (Compound 40)
Ac-SAIC(1)IWQDWGEHRC(1)[Sar]E-NH2 (Compound 41)
Ac-SAIC(1)IWQDWGEHRC(1)TEG-NH2 (Compound 42)
Ac-FC(1)IWQDWGEHRC(1)TGAE-NH2 (Compound 43)
Ac-EGSAIC(1)IWQDWGEHRC(1)[Sar]EGE-NH2 (Compound 44)
Ac-EGSAFC(1)IWQDWGEHRC(1)[Sar]E-NH2 (Compound 45)
Ac-EGSAIC(1)I[1-Me-Trp]QDWGEHRC(1)[Sar]E-NH2 (Compound 49)
Ac-EGSAIC(1)I[2-Nal]QDWGEHRC(1)[Sar]E-NH2 (Compound 50)
Ac-IC(1)I[1-Me-Trp]QDWGEHRC(1)TGAES-NH2 (Compound 51)
Ac-IC(1)I[2-Nal]QDWGEHRC(1)TGAES-NH2 (Compound 52)
Ac-EGSAFC(1)I[1-Me-Trp]QDWGEHRC(1)[Sar]E-NH2 (Compound 53)
Ac-EGSAYC(1)I[1-Me-Trp]QDWGEHRC(1)[Sar]E-NH2 (Compound 54)
Ac-EGSAIC(1)IWQDWGEHRC(1)TE-NH2 (Compound 55)
Ac-EGSAFC(1)I[1-Nal]QDWGEHRC(1)TE-NH2 (Compound 56)
Ac-EGSAFC(1)I[1-Me-Trp]QDWGEHRC(1)TE-NH2 (Compound 57)
Ac-EGSAFC(1)I[1-Me-Trp]QDWGEHRC(1)EGE-NH2 (Compound 58)
Ac-EGSAYC(1)I[1-Me-Trp]QDWGEHRC(1)TE-NH2 (Compound 59)
Ac-EGSAFC(1)I[2-Nal]QDWGEHRC(1)TE-NH2 (Compound 60)
Ac-FC(1)I[1-Me-Trp]QDWGEHRC(1)TGAES-NH2 (Compound 61)
Ac-YC(1)I[1-Me-Trp]QDWGEHRC(1)TGAES-NH2 (Compound 62)
Ac-FC(1)I[1-Nal]QDWGEHRC(1)TGAES-NH2 (Compound 63)
Ac-FC(1)I[2-Nal]QDWGEHRC(1)TGAES-NH2 (Compound 64)
Ac-YC(1)I[2-Nal]QDWGEHRC(1)TGAES-NH2 (Compound 65)
Ac-YC(1)IWQDWGEHRC(1)TGAES-NH2 (Compound 66)
Ac-SEFC(1)I[1-Me-Trp]QDWGEHRC(1)TGAES-NH2 (Compound 67)
Ac-YC(1)I[1-Me-Trp]QDWGEHRC(1)TEAGS-NH2 (Compound 68)
Ac-YC(1)I[1-Me-Trp]QDWGEHRC(1)TESGA-NH2 (Compound 69)
Ac-EGSAYC(1)I[1-Me-Trp]QEWGEHRC(1)[Sar]E-NH2 (Compound 70)
Ac-SEYC(1)I[1-Me-Trp]QDWGEHRC(1)[Sar]EA-NH2 (Compound 71)
Ac-FC(1)I[1-Me-Trp]QDW[Sar]EHRC(1)TGAES-NH2 (Compound 72)
H-{d}YFC(1)I[1-Me-Trp]QDW[Sar]EHRC(1)TGAES-NH2 (Compound 73)
Ac-SEFC(1)I[1-Me-Trp]QDWGEHRC(1)[Sar]GAES-NH2 (Compound 74)
Ac-SEFC(1)I[1-Me-Trp]QDWGEHRC(1)[Sar]EA-NH2 (Compound 75)
Ac-SEFC(1)I[1-Me-Trp]QDW[Sar]EHRC(1)[Sar]EA-NH2 (Compound 76)
Ac-SEFC(1)I[1-Me-Trp]QDW[Sar]EHRC(1)TEA-NH2 (Compound 77)
Ac-SEFC(1)I[1-Me-Trp]QDWGEHRC(1)[Sar]E-NH2 (Compound 78)
Ac-SEFC(1)I[1-Me-Trp]QDW[Sar]EHRC(1)[Sar]E-NH2 (Compound 79)
Ac-EFC(1)I[1-Me-Trp]QDWGEHRC(1)[Sar]EA-NH2 (Compound 80)
Ac-SE[Sar]C(1)I[1-Me-Trp]QDWGEHRC(1)[Sar]EA-NH2 (Compound 81)
Ac-SE[Sar]C(1)I[1-Me-Trp]QDWGEHRC(1)TEA-NH2 (Compound 82)
Ac-SEFC(1)I[1-Me-Trp]QEWGEHRC(1)[Sar]EA-NH2 (Compound 83)
Ac-SEFC(1)I[1-Me-Trp]QDWGEHRC(1)SEA-NH2 (Compound 84)
Ac-EFC(1)I[1-Me-Trp]QDWGEHRC(1)ES-NH2 (Compound 85)
Ac-SEFC(1)I[1-Me-Trp]QDWGEHKC(1)[Sar]EA-NH2 (Compound 86)
Ac-GEFC(1)I[1-Me-Trp]QDWGEHRC(1)[Sar]EA-NH2 (Compound 87)
Ac-GE[Sar]C(1)I[1-Me-Trp]QDWGEHRC(1)TEA-NH2 (Compound 88)
Ac-SE[Sar]C(1)I[1-Me-Trp]QEW[Sar]EHRC(1)TEA-NH2 (Compound 89)
Ac-SE[Sar]C(1)I[1-Me-Trp]QEWGEHRC(1)[Sar]EA-NH2 (Compound 90)
H-{d}Y[Sar]C(1)I[1-Me-Trp]QDWGEHRC(1)TEA-NH2 (Compound 91)

25. A compstatin analogue according to claim 1 comprising a sequence selected from:
[K*]GSAIC(1)IWQDWGEHRC(1)TEGE (Compound 100)
ASGEYC(1)I[1-Me-Trp]QDWGEHRC(1)[Sar]EGE-[K*] (Compound 113)
EFC(1)I[1-Me-Trp]QDWGEHRC(1)EGE-[K*] (Compound 134)
EGSAIC(1)IWQDWGEHRC(1)TEG-[K*] (Compound 101)
EGSAYC(1)I[1-Me-Trp]QDWGEH[K*]C(1)[Sar]E (Compound 103)
EGSAYC(1)I[1-Me-Trp]QDWGEHRC(1)[Sar]EG-[K*] (Compound 104)
EGSAYC(1)I[1-Me-Trp]QDWGEHRC(1)[Sar]EGE-[K*] (Compound 109)
EGSAYC(1)I[1-Me-Trp]QDWGEHRC(1)[Sar]EGK-[K*] (Compound 110)
EGSAYC(1)I[1-Me-Trp]QDWGEHRC(1)[Sar]EK[γGlu]-[K*] (Compound 111)
FC(1)I[1-Me-Trp]QDWGEHRC(1)TGAES[K*] (Compound 102)
IC(1)IWQDWGEHRC(1)TEG-[K*] (Compound 92)
IC(1)IWQDWGEHRC(1)TEGE-[K*] (Compound 94)
SAYC(1)I[1-Me-Trp]QDWGEHRC(1)[Sar]E-[K*] (Compound 105)
SEFC(1)I[1-Me-Trp]QDWGEHRC(1)[Sar]EGA-[K*] (Compound 119)
SEFC(1)I[1-Me-Trp]QDWGEHRC(1)[Sar]EGE[Peg3][Peg3]-[K*] (Compound 123)
SEFC(1)I[1-Me-Trp]QDWGEHRC(1)[Sar]EGEGGG-[K*] (Compound 129)
SEFC(1)I[1-Me-Trp]QDWGEHRC(1)[Sar]EGE[Peg3]-[K*] (Compound 138)
SEFC(1)I[1-Me-Trp]QDWGEHRC(1)[Sar]EGE[Peg3]ES-[K*] (Compound 140)
SEFC(1)I[1-Me-Trp]QDWGEHRC(1)[Sar]EGE[Peg3][Peg3]-[K*] (Compound 127)
SEFC(1)I[1-Me-Trp]QDWGEHRC(1)[Sar]EGESES-[K*] (Compound 139)
SEFC(1)I[1-Me-Trp]QDWGEHRC(1)[Sar]EK[γGlu]GGG-[K*] (Compound 132)
SEFC(1)I[1-Me-Trp]QDWGEHRC(1)TEGE-[8-aminooctanoyl]-[K*] (Compound 136)
SEFC(1)I[1-Me-Trp]QDWGEHRC(1)TEGE-[8-aminooctanoyl]-E-[K*] (Compound 137)
SEFC(1)I[1-Me-Trp]QDWGEHRC(1)TEGEGGG-[K*] (Compound 130)
SEFC(1)I[1-Me-Trp]QDWGEHRC(1)TEGE[Peg3]ES-[K*] (Compound 142)
SEFC(1)I[1-Me-Trp]QDWGEHRC(1)TEGE[Peg3][Peg3 ]-[K*] (Compound 126)
SEFC(1)I[1-Me-Trp]QDWGEHRC(1)TEK[γGlu]GGG-[K*] (Compound 133)
SEFC(1)I[1-Me-Trp]QDWGEHRC(1)TGAES-[K*] (Compound 135)
SEFC(1)I[1-Me-Trp]QEWGEHRC(1)[Sar]EGA-[K*] (Compound 120)
SEFC(1)I[1-Me-Trp]QEWGEHRC(1)[Sar]EGE[Peg3][Peg3][K*] (Compound 124)
SEYC(1)I[1-Me-Trp]QDWGEHRC(1)[Sar]EGA-[K*] (Compound 112)
SEYC(1)I[1-Me-Trp]QDWGEHRC(1)[Sar]EGE[Peg3][Peg3]-[K*] (Compound 117)
SEYC(1)I[1-Me-Trp]QDWGEHRC(1)[Sar]EGE-[K*] (Compound 114)
SEYC(1)I[1-Me-Trp]QEW[Sar]EHRC(1)[Sar]EK[γGlu]A[K*] (Compound 121)
SEYC(1)I[1-Me-Trp]QEWGEHRC(1)[Sar]EGA[K*] (Compound 122)
SEYC(1)I[1-Me-Trp]QEWGEHRC(1)[Sar]EGE[Peg3][Peg3]-[K*] (Compound 125)
EGSEYC(1)I[1-Me-Trp]QDWGEHRC(1)[Sar]E (Compound 107)
ESSAIC(1)IWQDWGEHRC(1)TEGE (Compound 99)
SEFC(1)I[1-Me-Trp]QDWGEHRC(1)[Sar]EGE[Peg3][Peg3][Peg3]-[K*] (Compound 143)
SEFC(1)I[1-Me-Trp]QDW[Sar]EHRC(1)[Sar]E[Peg3][Peg3]-[K*] (Compound 144)
EFC(1)I[1-Me-Trp]QDWGEHRC(1)[Sar]EA[Peg3][Peg3]-[K*] (Compound 145)

26. A compstatin analogue according to claim 25 comprising a sequence selected from:
Ac-[K*]GSAIC(1)IWQDWGEHRC(1)TEGE-NH2 (Compound 100)
Ac-ASGEYC(1)I[1-Me-Trp]QDWGEHRC(1)[Sar]EGE-[K*]-[NH2] (Compound 113)
Ac-EFC(1)I[1-Me-Trp]QDWGEHRC(1)EGE-[K*]-[NH2] (Compound 134)
Ac-EGSAIC(1)IWQDWGEHRC(1)TEG-[K*]-[NH2] (Compound 101)
Ac-EGSAYC(1)I[1-Me-Trp]QDWGEH[K*]C(1)[Sar]E-[NH2] (Compound 103)
Ac-EGSAYC(1)I[1-Me-Trp]QDWGEHRC(1)[Sar]EG-[K*]-NH2 (Compound 104)
Ac-EGSAYC(1)I[1-Me-Trp]QDWGEHRC(1)[Sar]EGE-[K*]-NH2 (Compound 109)
Ac-EGSAYC(1)I[1-Me-Trp]QDWGEHRC(1)[Sar]EGK-[K*]-NH2 (Compound 110)
Ac-EGSAYC(1)I[1-Me-Trp]QDWGEHRC(1)[Sar]EK[γGlu]-[K*]-NH2 (Compound 111)
Ac-FC(1)I[1-Me-Trp]QDWGEHRC(1)TGAES-[K*]-NH2 (Compound 102)
Ac-IC(1)IWQDWGEHRC(1)TEG-[K*]-NH2 (Compound 92, 93, 95, 96, 98)
Ac-IC(1)IWQDWGEHRC(1)TEGE-[K*]-NH2 (Compound 94, 97)
Ac-SAYC(1)I[1-Me-Trp]QDWGEHRC(1)[Sar]E-[K*]-NH2 (Compound 105, 106)
Ac-SEFC(1)I[1-Me-Trp]QDWGEHRC(1)[Sar]EGA-[K*]-NH2 (Compound 119)
Ac-SEFC(1)I[1-Me-Trp]QDWGEHRC(1)[Sar]EGE[Peg3][Peg3]-[K*]-NH2 (Compound 123)
Ac-SEFC(1)I[1-Me-Trp]QDWGEHRC(1)[Sar]EGEGGG-[K*]-NH2 (Compound 129)
Ac-SEFC(1)I[1-Me-Trp]QDWGEHRC(1)[Sar]EGE[Peg3]-[K*]-NH2 (Compound 138)
Ac-SEFC(1)I[1-Me-Trp]QDWGEHRC(1)[Sar]EGE[Peg3]ES-[K*]-NH2 (Compound 140)
Ac-SEFC(1)I[1-Me-Trp]QDWGEHRC(1)[Sar]EGE[Peg3][Peg3]-[K*]-NH2 (Compound 127, 128)
Ac-SEFC(1)I[1-Me-Trp]QDWGEHRC(1)[Sar]EGESES-[K*]-NH2 (Compound 139, 141)
Ac-SEFC(1)I[1-Me-Trp]QDWGEHRC(1)[Sar]EK[γGlu]GGG-[K*]-NH2 (Compound 132)
Ac-SEFC(1)I[1-Me-Trp]QDWGEHRC(1)TEGE[8-aminooctanoyl]-[K*]-NH2 (Compound 136)
Ac-SEFC(1)[1-Me-Trp]QDWGEHRC(1)TEGE[8-aminooctanoyl]E-[K*]-NH2 (Compound 137)
Ac-SEFC(1)I[1-Me-Trp]QDWGEHRC(1)TEGEGGG-[K*]-NH2 (Compound 130, 131)
Ac-SEFC(1)I[1-Me-Trp]QDWGEHRC(1)TEGE[Peg3]ES-[K*]-NH2 (Compound 142)
Ac-SEFC(1)I[1-Me-Trp]QDWGEHRC(1)TEGE[Peg3][Peg3]-[K*]-NH2 (Compound 126)
Ac-SEFC(1)I[1-Me-Trp]QDWGEHRC(1)TEK[γGlu]GGG-[K*]-NH2 (Compound 133)
Ac-SEFC(1)I[1-Me-Trp]QDWGEHRC(1)TGAES-[K*]-NH2 (Compound 135)
Ac-SEFC(1)I[1-Me-Trp]QEWGEHRC(1)[Sar]EGA-[K*]-NH2 (Compound 120)
Ac-SEFC(1)I[1-Me-Trp]QEWGEHRC(1)[Sar]EGE[Peg3][Peg3]-[K*]-NH2 (Compound 124)
Ac-SEYC(1)I[1-Me-Trp]QDWGEHRC(1)[Sar]EGA-[K*]-NH2 (Compound 112, 118)
Ac-SEYC(1)I[1-Me-Trp]QDWGEHRC(1)[Sar]EGE[Peg3][Peg3]-[K*]-NH2 (Compound 117)
Ac-SEYC(1)I[1-Me-Trp]QDWGEHRC(1)[Sar]EGE-[K*]-NH2 (Compound 114, 115, 116)
Ac-SEYC(1)I[1-Me-Trp]QEW[Sar]EHRC(1)[Sar]EK[γGlu]A-[K*]-NH2 (Compound 121)
Ac-SEYC(1)I[1-Me-Trp]QEWGEHRC(1)[Sar]EGA-[K*]-NH2 (Compound 122)
Ac-SEYC(1)I[1-Me-Trp]QEWGEHRC(1)[Sar]EGE[Peg3][Peg3]-[K*]-NH2 (Compound 125)
Φ-EGSEYC(1)I[1-Me-Trp]QDWGEHRC(1)[Sar]E-NH2 (Compound 107, 108)
Φ-ESSAIC(1)IWQDWGEHRC(1)TEGE-NH2 (Compound 99)
Ac-SEFC(1)I[1-Me-Trp]QDWGEHRC(1)[Sar]EGE[Peg3][Peg3][Peg3]-[K*]-NH2 (Compound 143)
Ac-SEFC(1)I[1-Me-Trp]QDW[Sar]EHRC(1)[Sar]E[Peg3][Peg3]-[K*]-NH2 (Compound 144)
Ac-EFC(1)I[1-Me-Trp]QDWGEHRC(1)[Sar]EA[Peg3][Peg3]-[K*]-NH2 (Compound 145)

27. A compstatin analogue according to claim 1 which comprises a lipophilic group Φ, and wherein the lipophilic group Φ is Z¹— or Z¹—Z²—; wherein
Z¹ is A—C₁₂₋₂₂alkylene—(CO)—;
where A is H or —COOH, and wherein the akylene may be linear or branched and may be saturated or unsaturated, and may optionally incorporate a phenylene or piperazinylene moiety in its length; and
Z² is a sequence of 1 to 6 residues of compounds selected from γ-Glu, E, K, Orn, S, T, A, β-Ala, G, P, V, L, I, Y, Q, N, Dapa, Gaba, or Aib, or a correspdoning D form thereof, 5-aminopentanoyl, 6-aminohexanoyl, 7-aminoheptanoyl, 8-aminooctanoyl, 9-aminononanoyl, and 10-aminodecanoyl. 8-amino-3,6-dioxaoctanoic acid (Peg3), 11-amino-3,6,9-trioxaundecanoic acid (Peg4) and (piperazine-1-yl)-carboxylic acid.

28. A compstatin analogue according to claim 27 wherein Z¹ is selected from:
Dodecanoyl i.e. H—(CH₂)₁₁—(CO)—;
Tetradecanoyl i.e. H—(CH₂)₁₃—(CO)—;
Hexadecanoyl, i.e. H—(CH₂)₁₅—(CO)—;
13-carboxytridecanoyl, i.e. HOOC—(CH₂)₁₂—(CO)—;
15-carboxypentadecanoyl, i.e. HOOC—(CH₂)₁₄—(CO)—;
17-carboxyheptadecanoyl, i.e. HOOC—(CH₂)₁₆—(CO)—;
19-carboxynonadecanoyl, i.e. HOOC—(CH₂)₁₈—(CO)—; or
21-carboxyheneicosanoyl, i.e. HOOC—(CH₂)₂₀—(CO)—

29. A compstatin analogue according to claim 27 wherein Z² is selected from:
[γGlu],
[γGlu][Peg3][Peg3]—;
[(Piperazine-1-yl)-acetyl][Peg3][Peg3];
[γGlu]G[γGlu];
[γGlu]K[γGlu];
[γGlu]KG[γGlu]; or
[γGlu]G[Peg3][γGlu][Peg3].

30. A compstatin analogue according to claim 27 wherein Z¹— or Z¹—Z²— is selected from:
15-carboxy-pentadecanoyl;
15-carboxy-pentadecanoyl[γGlu]—,
15-carboxy-pentadecanoyl[γGlu][Peg3][Peg3]—;
19-carboxy-nonadecanoyl[γGlu][Peg3][Peg3]—;
15-carboxy-pentadecanoyl-[(Piperazine-1-yl)-acetyl][Peg3][Peg3]);
17-carboxy-heptadecanoyl[γGlu]G[γGlu];
17-carboxy-heptadecanoyl[γGlu]K[γGlu];
17-carboxy-heptadecanoyl[γGlu]KG[γGlu];
17-carboxy-heptadecanoyl[γGlu]G(Peg3)[γGlu]—(Peg3);
15-carboxy-hexadecanoyl[γGlu]G[γGlu];
17-carboxy-heptadecanoyl;
17-carboxy-heptadecanoyl[γGlu]]
19-carboxy-nonadecanoyl[γGlu]G[γGlu]; and
17-carboxy-heptadecanoyl[γGlu][Peg3][Peg3].

31. A compstatin analogue according to claim 1 which is:
Ac-IC(1)IWQDWGEHRC(1)TEG-K([15-carboxy-pentadecanoyl][γGlu])-NH2 (Compound 92)
Ac-IC(1)IWQDWGEHRC(1)TEG-K([15-carboxy-pentadecanoyl][γGlu][Peg3][Peg3])-NH2 (Compound 93)
Ac-IC(1)IWQDWGEHRC(1)TEGE-K([15-carboxy-pentadecanoyl][γGlu][Peg3][Peg3])-NH2 (Compound 94)
Ac-IC(1)IWQDWGEHRC(1)TEG-K((15-carboxy-pentadecanoyl)-[(Piperazine-1-yl)-acetyl][Peg3][Peg3])-NH2 (Compound 95)
Ac-IC(1)IWQDWGEHRC(1)TEG-K([17-carboxy-heptadecanoyl][γGlu][Peg3][Peg3])-NH2 (Compound 96)
Ac-IC(1)IWQDWGEHRC(1)TEGE-K([17-carboxy-heptadecanoyl][γGlu][Peg3][Peg3])-NH2 (Compound 97)
Ac-IC(1)IWQDWGEHRC(1)TEG-K([19-carboxy-nonadecanoyl][γGlu][Peg3][Peg3])-NH2 (Compound 98)
[15-Carboxy-pentadecanoyl]-ESSAIC(1)IWQDWGEHRC(1)TEGE-NH2 (Compound 99)
Ac-[K([15-carboxy-pentadecanoyl]-[γGlu][Peg3][Peg3])]GSAIC(1)IWQDWGEHRC(1)TEGE-NH2 (Compound 100)
Ac-EGSAIC(1)IWQDWGEHRC(1)TEG-K([15-carboxy-pentadecanoyl][γGlu])-NH2 (Compound 101)
Ac-FC(1)I[1-Me-Trp]QDWGEHRC(1) TGAES-K([15-carboxy-pentadecanoyl][vGlu][Peg3][Peg3])-NH2 (Compound 102)
Ac-EGSAYC(1)I[1-Me-Trp]QDWGEH-K([15-carboxy-pentadecanoyl][γGlu][Peg3][Peg3])-C(1)[Sar]E-NH2 (Compound 103)
Ac-EGSAYC(1)I[1-Me-Trp]QDWGEHRC(1)[Sar]EG-K([15-carboxy-pentadecanoyl][γGlu][Peg3][Peg3])-NH2 (Compound 104)
Ac-SAYC(1)I[1-Me-Trp]QDWGEHRC(1)[Sar]E-K([17-carboxy-heptadecanoyl][γGlu]KG[γGlu])-NH2 (Compound 105)
Ac-SAYC(1)I[1-Me-Trp]QDWGEHRC(1)[Sar]E-K([17-carboxy-heptadecanoyl][γGlu]G[γGlu])-NH2 (Compound 106)
[15-Carboxy-pentadecanoyl]-EGSEYC(1)I[1-Me-Trp]QDWGEHRC(1)[Sar]E-NH2 (Compound 107)
[17-Carboxy-heptadecanoyl]-EGSEYC(1)I[1-Me-Trp]QDWGEHRC(1)[Sar]E-NH2 (Compound 108)
Ac-EGSAYC(1)I[1-Me-Trp]QDWGEHRC(1)[Sar]EGE-K([17-carboxy-heptadecanoyl]-[γGlu]G[γGlu])-NH2 (Compound 109)
Ac-EGSAYC(1)I[1-Me-Trp]QDWGEHRC(1)[Sar]EGK-K([17-carboxy-heptadecanoyl][γGlu]G[γGlu])-NH2 (Compound 110)
Ac-EGSAYC(1)I[1-Me-Trp]QDWGEHRC(1)[Sar]EK[γGlu]-K([17-carboxy-heptadecanoyl][γGlu][Peg3][Peg3])]-NH2 (Compound 111)
Ac-SEYC(1)I[1-Me-Trp]QDWGEHRC(1)[Sar]EGA-K([17-carboxy-heptadecanoyl][γGlu]-G[γGlu])]-NH2 (Compound 112)
Ac-ASGEYC(1)I[1-Me-Trp]QDWGEHRC(1)[Sar]EGE-K([17-carboxy-heptadecanoyl][Glu]-G[γGlu])-NH2 (Compound 113)
Ac-SEYC(1)I[1-Me-Trp]QDWGEHRC(1)[Sar]EGE-K([17-carboxy-heptadecanoyl][γGlu]-G[γGlu])-NH2 (Compound 114)
Ac-SEYC(1)I[1-Me-Trp]QDWGEHRC(1)[Sar]EGK-K([17-carboxy-heptadecanoyl][γGlu]-G[γGlu])-NH2 (Compound 115)
Ac-SEYC(1)I[1-Me-Trp]QDWGEHRC(1)[Sar]EGE-K([17-carboxy-heptadecanoyl][γGlu]-K[γGlu])-NH2 (Compound 116)
Ac-SEYC(1)I[1-Me-Trp]QDWGEHRC(1)[Sar]EGE[Peg3][Peg3]-K([17-carboxy-heptadecanoyl][γGlu]G[γGlu])-NH2 (Compound 117)
Ac-SEYC(1)I[1-Me-Trp]QDWGEHRC(1)[Sar]EGA-K([17-carboxy-heptadecanoyl][γGlu]-G[Peg3][γGlu][Peg3])-NH2 (Compound 118)
Ac-SEFC(1)I[1-Me-Trp]QDWGEHRC(1)[Sar]EGA-K([17-carboxy-heptadecanoyl][γGlu]-G[Peg3][γGlu][Peg3])-NH2 (Compound 119)
Ac-SEFC(1)I[1-Me-Trp]QEWGEHRC(1)[Sar]EGA-K([17-carboxy-heptadecanoyl][γGlu]-G[Peg3][γGlu][Peg3])-NH2 (Compound 120)
Ac-SEYC(1)I[1-Me-Trp]QEW[Sar]EHRC(1)[Sar]EK[γGlu]A-K([17-carboxy-heptadecanoyl][γGlu]G[Peg3][γGlu]-[Peg3])-NH2 (Compound 121)
Ac-SEYC(1)I[1-Me-Trp]QEWGEHRC(1)[Sar]EGA-K([17-carboxy-heptadecanoyl][γGlu]-G-[Peg3][γGlu][Peg3])-NH2 (Compound 122)
Ac-SEFC(1)I[1-Me-Trp]QDWGEHRC(1)[Sar]EGE[Peg3][Peg3]-K([17-carboxy-heptadecanoyl][γGlu]G[γGlu])-NH2 (Compound 123)
Ac-SEFC(1)I[1-Me-Trp]QEWGEHRC(1)[Sar]EGE[Peg3][Peg3]-K([17-carboxy-heptadecanoyl][γGlu]G[γGlu])-NH2 (Compound 124)
Ac-SEYC(1)I[1-Me-Trp]QEWGEHRC(1)[Sar]EGE[Peg3][Peg3]-K([17-carboxy-heptadecanoyl][γGlu]G[γGlu])-NH2 (Compound 125)
Ac-SEFC(1)I[1-Me-Trp]QDWGEHRC(1)TEGE[Peg3][Peg3]-K([17-carboxy-heptadecanoyl][γGlu]G[γGlu])-NH2 (Compound 126)
Ac-SEFC(1)I[1-Me-Trp]QDWGEHRC(1)[Sar]EGE[Peg3][Peg3]-[K[(15-carboxy-pentadecanoyl)[γGlu]G[γGlu])-NH2 (Compound 127)
Ac-SEFC(1)I[1-Me-Trp]QDWGEHRC(1)[Sar]EGE[Peg3][Peg3]-[K[(19-carboxy-nonadecanoyl)[γGlu]G[γGlu])-NH2 (Compound 128)
Ac-SEFC(1)I[1-Me-Trp]QDWGEHRC(1)[Sar]EGEGGG-K([17-carboxy-heptadecanoyl]-[γGlu]G[γGlu])-NH2 (Compound 129)
Ac-SEFC(1)I[1-Me-Trp]QDWGEHRC(1)TEGEGGG-K([17-carboxy-heptadecanoyl]-[γGlu]G[γGlu])-NH2 (Compound 130)
Ac-SEFC(1)I[1-Me-Trp]QDWGEHRC(1)TEGEGGG-K([15-carboxy-pentadecanoyl]-[γGlu]G[γGlu])-NH2 (Compound 131)
Ac-SEFC(1)I[1-Me-Trp]QDWGEHRC(1)[Sar]EK[γGlu]GGG-K([17-carboxy-heptadecanoyl][γGlu]G[γGlu])-NH2 (Compound 132)
Ac-SEFC(1)I[1-Me-Trp]QDWGEHRC(1) TEK[γGlu]GGG-K([17-carboxy-heptadecanoyl]-[γGlu]G[γGlu])-NH2 (Compound 133)
Ac-EFC(1)I[1-Me-Trp]QDWGEHRC(1)EGE-K([17-carboxy-heptadecanoyl][γGlu]G[γGlu])-NH2 (Compound 134)
Ac-SEFC(1)I[1-Me-Trp]QDWGEHRC(1)TGAES-K([15-carboxy-hexadecanoyl][γGlu]G[γGlu])-NH2 (Compound 135)
Ac-SEFC(1)I[1-Me-Trp]QDWGEHRC(1)TEGE[8-aminooctanoyl]-K([17-carboxy-heptadecanoyl][γGlu]G[γGlu])-NH2 (Compound 136)
Ac-SEFC(1)I[1-Me-Trp]QDWGEHRC(1)TEGE[8-aminooctanoyl]E-K([17-carboxy-heptadecanoyl][γGlu]G[γGlu])-NH2 (Compound 137)
Ac-SEFC(1)I[1-Me-Trp]QDWGEHRC(1)[Sar]EGE[Peg3]-K([17-carboxy-heptadecanoyl][γGlu]-G[γGlu])-NH2 (Compound 138)
Ac-SEFC(1)I[1-Me-Trp]QDWGEHRC(1)[Sar]EGESES-K([17-carboxy-heptadecanoyl]-[γGlu]G[γGlu])-NH2 (Compound 139)
Ac-SEFC(1)I[1-Me-Trp]QDWGEHRC(1)[Sar]EGE[Peg3]ES-K([17-carboxy-heptadecanoyl][γGlu]G[γGlu])-NH2 (Compound 140)
Ac-SEFC(1)I[1-Me-Trp]QDWGEHRC(1)[Sar]EGESES-K([17-carboxy-heptadecanoyl][γGlu])-NH2 (Compound 141)
Ac-SEFC(1)I[1-Me-Trp]QDWGEHRC(1)TEGE[Peg3]ES-K([17-carboxy-heptadecanoyl][γGlu])-NH2 (Compound 142)
Ac-SEFC(1)I[1-Me-Trp]QDWGEHR[C(1)[Sar]EGE[Peg3][Peg3][Peg3]-K([17-carboxy-heptadecanoyl][γGlu]G[γGlu])-NH2 (Compound 143)
Ac-SEFC(1)I[1-Me-Trp]QDW[Sar]EHRC(1)[Sar]E[Peg3][Peg3]-K([17-carboxy-heptadecanoyl][γGlu]G[γGlu])-NH2 (Compound 144)
Ac-EF[C(1)I[1-Me-Trp]QDWGEHRC(1)[Sar]EA[Peg3][Peg3]-K([17-carboxy-heptadecanoyl][γGlu]G[γGlu])-NH2 (Compound 145)

32. A composition comprising a compstatin analogue according to claim 1, or a pharmaceutically acceptable salt or solvate thereof, in admixture with a carrier.

33. (canceled)

34. A pharmaceutical composition comprising a compstatin analogue according to claim 1, or a pharmaceutically acceptable salt thereof, in admixture with a pharmaceutically acceptable carrier, excipient or vehicle.

35-39. (canceled)

40. A method of inhibiting complement activation for treating a subject in need thereof, the method comprising administering to the subject a compstatin analogue, or a pharmaceutically acceptable salt thereof, according to claim 1 thereby to inhibit complement activation in the subject.

41. (canceled)

42. An ex vivo method of inhibiting complement activation during extracorporeal shunting of a physiological fluid, the method comprising contacting the physiological fluid with a compstatin analogue, or a pharmaceutically acceptable salt thereof, according to claim 1, thereby inhibiting complement activation.

43-44. (canceled)


SEQUENCE LISTING (334 sequences)

SEQ ID NO: 1 - protein, 13 aa, synthetic construct
  REGION 1..13: note = Description of Artificial Sequence: Synthetic peptide
  REGION 1..13: note = core compstatin peptide
  DISULFID 2..12: note = Intrachain disulfide bond
ICVVQDWGHHRCT

SEQ ID NO: 2 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, F, V, Y, 1-Me-Trp, D-Trp, N-Me-Trp, 1-For-Trp, 1-Nal, 2-Nal, 5-Me-Trp, Bpa or 2-Igl
  VARIANT 12: note = E, K or D
  VARIANT 14: note = G or Sar
  VARIANT 15: note = H, A, E, D, K, R or S
  VARIANT 17: note = R, S or K
  VARIANT 19: note = T, S, E, F, H, K, Sar, G, I, D, N-Me-Ile or N-Me-Thr
  VARIANT 20..27: note = A, E, G, L, K, F, P, S, T, W, Y, R, V, Sar, epsilonLys, gammaGlu, betaAsp, or betaAla, or a corresponding D form thereof or Peg3, Peg4, 8-aminooctanoyl derivatives thereof or may be absent
  VARIANT 20..27: note = This region may encompass 1 to 8 amino acid residues
XXXXXXXCIXQXWXXHXCXXXXXXXXX

SEQ ID NO: 3 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 2-Nal, 1-Nal or 1-Me-Trp
  VARIANT 12: note = E or D
  VARIANT 14: note = G or Sar
  VARIANT 15: note = A, E, D, K or S
  VARIANT 17: note = R, S or K
  VARIANT 19: note = T, S, E, I, Sar, K, G or N-Me-Ile
  VARIANT 20..27: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..27: note = This region may encompass 1 to 8 amino acid residues
XXXXXXXCIXQXWXXHXCXXXXXXXXX

SEQ ID NO: 4 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y,1-Nal, 2-Nal or 1-Me-Trp
  VARIANT 12: note = E or D
  VARIANT 15: note = A, E, D, K or S
  VARIANT 17: note = R, S or K
  VARIANT 19: note = T, I, S, E, K or Sar
  VARIANT 20..27: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..27: note = This region may encompass 1 to 8 amino acid residues
XXXXXXXCIXQXWGXHXCXXXXXXXXX

SEQ ID NO: 5 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 1-Nal, 2-Nal or 1-Me-Trp
  VARIANT 12: note = E or D
  VARIANT 15: note = A, E, D, K or S
  VARIANT 19: note = T, S, E or Sar
  VARIANT 20..27: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..27: note = This region may encompass 1 to 8 amino acid residues
XXXXXXXCIXQXWGXHRCXXXXXXXXX

SEQ ID NO: 6 - protein, 25 aa, synthetic construct
  REGION 1..25: note = Description of Artificial Sequence: Synthetic peptide
  REGION 1..25: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = Y or F
  DISULFID 8..18: note = Intrachain disulfide bond
  VARIANT 10: note = W, Y, or 1-Me-Trp
  VARIANT 12: note = E or D
  VARIANT 15: note = A, E or K
  VARIANT 19: note = T, E or Sar
  VARIANT 20..25: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..25: note = This region may encompass 1 to 6 amino acid residues
XXXXXXXCIXQXWGXHRCXXXXXXX

SEQ ID NO: 7 - protein, 25 aa, synthetic construct
  REGION 1..25: note = Description of Artificial Sequence: Synthetic peptide
  REGION 1..25: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = Y or F
  DISULFID 8..18: note = Intrachain disulfide bond
  MOD_RES 10: note = 1-methyl-tryptophan
  VARIANT 12: note = E or D
  VARIANT 19: note = T, E or Sar
  VARIANT 20..25: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..25: note = This region may encompass 1 to 6 amino acid residues
XXXXXXXCIXQXWGEHRCXXXXXXX

SEQ ID NO: 8 - protein, 27 aa, synthetic construct
  REGION 1..27: note = Description of Artificial Sequence: Synthetic peptide
  REGION 1..27: note = compstatin analogue
  VARIANT 1..6: note = A, E, G, K, K covalently linked to a lipophilic group via its side chain, S, Y, a corresponding D form thereof or may be absent
  VARIANT 1..6: note = This region may encompass 1 to 6 amino acid residues
  VARIANT 7: note = I, Y, F or Sar
  VARIANT 10: note = W, V, 1-Me-Trp, 1-Nal or 2-Nal
  VARIANT 12: note = E, K or D
  VARIANT 14: note = G or Sar
  VARIANT 15: note = H, A, E, D, K, R or S
  VARIANT 17: note = R, S, K or K covalently linked to a lipophilic group via its side chain
  VARIANT 19: note = T, S, E, Sar or N-Me-Ile
  VARIANT 20..27: note = A, E, G, K, K covalently linked to a lipophilic group via its side chain, P, S, Peg3, gammaGlu, 8-aminooctanoyl, a corresponding D form thereof or may be absent
  VARIANT 20..27: note = This region may encompass 1 to 8 amino acid residues
XXXXXXXCIXQXWXXHXCXXXXXXXXX

SEQ ID NO: 9 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, K covalently linked to a lipophilic group via its side chain, F, P, S, T, W, Y, R, V or Sar, a corresponding D form thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 2-Nal, 1-Nal or 1-Me-Trp
  VARIANT 12: note = E or D
  VARIANT 14: note = G or Sar
  VARIANT 15: note = A, E, D, K or S
  VARIANT 17: note = R, S or K covalently linked to a lipophilic group via its side chain
  VARIANT 19: note = T, S, E, I, Sar, K, G or N-Me-Ile
  VARIANT 20..27: note = A, E, G, L, K, K covalently linked to a lipophilic group via its side chain, F, P, S, T, W, Y, R, V, Sar, epsilonLys, gammaGlu, betaAsp, betaAla, or a corresponding D form thereof or Peg 3 or Peg4, or 8-aminooctanoyl derivatives thereof or may be absent
  VARIANT 20..27: note = This region may encompass 1 to 8 amino acid residues
XXXXXXXCIXQXWXXHXCXXXXXXXXX

SEQ ID NO: 10 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, K covalently linked to a lipophilic group via its side chain, F, P, S, T, W, Y, R, V or Sar, a corresponding D form thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 1-Nal, 2-Nal or 1-Me-Trp
  VARIANT 12: note = E or D
  VARIANT 15: note = A, E, D, K or S
  VARIANT 17: note = R, S or K covalently linked to a lipophilic group via its side chain
  VARIANT 19: note = T, I, S, E, K or Sar
  VARIANT 20..27: note = A, E, G, L, K, K covalently linked to a lipophilic group via its side chain F, P, S, T, W, Y, R, V, Sar, epsilonLys, gammaGlu, betaAsp, or betaAla, or a corresponding D form thereof or Peg3 or Peg4, or 8-aminooctanoyl derivatives thereof or may be absent
  VARIANT 20..27: note = This region may encompass 1 to 8 amino acid residues
XXXXXXXCIXQXWGXHXCXXXXXXXXX

SEQ ID NO: 11 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, K covalently linked to a lipophilic group via its side chain, F, P, S, T, W, Y, R, V or Sar, a corresponding D form thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, 1-Nal, 2-Nal or 1-Me-Trp
  VARIANT 12: note = E or D
  VARIANT 15: note = A, E, D, K or S
  VARIANT 19: note = T, S, E or Sar
  VARIANT 20..27: note = A, E, G, L, K, K covalently linked to a lipophilic group via its side chain, F, P, S, T, W, Y, R, V, Sar, epsilonLys, gammaGlu, betaAsp, or betaAla, or a corresponding D form thereof or Peg3 or Peg4, or 8-aminooctanoyl derivatives thereof or may be absent
  VARIANT 20..27: note = This region may encompass 1 to 8 amino acid residues
XXXXXXXCIXQXWGXHRCXXXXXXXXX

SEQ ID NO: 12 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, K covalently linked to a lipophilic group via its side chain, F, P, S, T, W, Y, R, V or Sar, a corresponding D form thereof or may be absent
  VARIANT 1..6: note = This region may encompass 1 to 6 amino acid residues
  VARIANT 7: note = Y or F
  DISULFID 8..18: note = Intrachain disulfide bond
  MOD_RES 10: note = 1-methyl-tryptophan
  VARIANT 12: note = E or D
  VARIANT 19: note = T, E or Sar
  VARIANT 20..27: note = A, E, G, L, K, K covalently linked to a lipophilic group via its side chain F, P, S, T, W, Y, R, V, Sar, epsilonLys, gammaGlu, betaAsp, or betaAla, or a corresponding D form thereof or Peg3 or Peg4, or 8-aminooctanoyl derivatives thereof or may be absent
  VARIANT 20..27: note = This region may encompass 1 to 8 amino acid residues
XXXXXXXCIXQXWGEHRCXXXXXXXXX

SEQ ID NO: 13 - protein, 13 aa, synthetic construct
  REGION 1..13: note = Description of Artificial Sequence: Synthetic peptide
  REGION 1..13: note = 13-mer peptide portion of the compstatin analogue
  MOD_RES 1: note = sarcosine
  DISULFID 2..12: note = Intrachain disulfide bond
  MOD_RES 4: note = 1-methyl-tryptophan
  MOD_RES 13: note = sarcosine
XCIWQDWGEHRCX

SEQ ID NO: 14 - protein, 13 aa, synthetic construct
  REGION 1..13: note = Description of Artificial Sequence: Synthetic peptide
  REGION 1..13: note = 13-mer peptide portion of the compstatin analogue
  MOD_RES 1: note = sarcosine
  DISULFID 2..12: note = Intrachain disulfide bond
  MOD_RES 4: note = 1-methyl-tryptophan
XCIWQDWGEHRCT

SEQ ID NO: 15 - protein, 13 aa, synthetic construct
  REGION 1..13: note = Description of Artificial Sequence: Synthetic peptide
  REGION 1..13: note = 13-mer peptide portion of the compstatin analogue
  MOD_RES 1: note = sarcosine
  DISULFID 2..12: note = Intrachain disulfide bond
  MOD_RES 4: note = 1-methyl-tryptophan
  MOD_RES 8: note = sarcosine
XCIWQEWXEHRCT

SEQ ID NO: 16 - protein, 13 aa, synthetic construct
  REGION 1..13: note = Description of Artificial Sequence: Synthetic peptide
  REGION 1..13: note = 13-mer peptide portion of the compstatin analogue
  MOD_RES 1: note = sarcosine
  DISULFID 2..12: note = Intrachain disulfide bond
  MOD_RES 4: note = 1-methyl-tryptophan
  MOD_RES 13: note = sarcosine
XCIWQEWGEHRCX

SEQ ID NO: 17 - protein, 13 aa, synthetic construct
  REGION 1..13: note = Description of Artificial Sequence: Synthetic peptide
  REGION 1..13: note = 13-mer peptide portion of the compstatin analogue
  MOD_RES 1: note = sarcosine
  DISULFID 2..12: note = Intrachain disulfide bond
XCIWQDWGEHRCT

SEQ ID NO: 18 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 8: note = sarcosine
  MOD_RES 13: note = sarcosine
FCIWQDWXEHRCX

SEQ ID NO: 19 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 8: note = sarcosine
FCIWQDWXEHRCT

SEQ ID NO: 20 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 13: note = sarcosine
FCIWQDWGEHKCX

SEQ ID NO: 21 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 13: note = sarcosine
FCIWQDWGEHRCX

SEQ ID NO: 22 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
FCIWQDWGEHRCE

SEQ ID NO: 23 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
FCIWQDWGEHRCS

SEQ ID NO: 24 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
FCIWQDWGEHRCT

SEQ ID NO: 25 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 13: note = sarcosine
FCIWQEWGEHRCX

SEQ ID NO: 26 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-naphathalin
FCIXQDWGEHRCT

SEQ ID NO: 27 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 2-naphathalin
FCIXQDWGEHRCT

SEQ ID NO: 28 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 13: note = sarcosine
FCIWQDWGEHRCX

SEQ ID NO: 29 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
FCIWQDWGEHRCT

SEQ ID NO: 30 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 8: note = sarcosine
  MOD_RES 13: note = N-methyl-isoleucine
ICIWQDWXAHRCI

SEQ ID NO: 31 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 13: note = sarcosine
ICIWQDWGEHRCX

SEQ ID NO: 32 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
ICIWQDWGEHRCT

SEQ ID NO: 33 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 2-naphathalin
  MOD_RES 13: note = sarcosine
ICIXQDWGEHRCX

SEQ ID NO: 34 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGAHRCE

SEQ ID NO: 35 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGAHRCT

SEQ ID NO: 36 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGAHSCT

SEQ ID NO: 37 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGDHRCT

SEQ ID NO: 38 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 13: note = sarcosine
ICIWQDWGEHRCX

SEQ ID NO: 39 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGEHRCE

SEQ ID NO: 40 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGEHRCS

SEQ ID NO: 41 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGEHRCT

SEQ ID NO: 42 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGEHSCT

SEQ ID NO: 43 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGKHRCT

SEQ ID NO: 44 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGRHRCT

SEQ ID NO: 45 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQDWGSHRCT

SEQ ID NO: 46 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQEWGEHRCT

SEQ ID NO: 47 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQKWGAHRCT

SEQ ID NO: 48 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
ICIWQKWGEHRCT

SEQ ID NO: 49 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 13: note = sarcosine
YCIWQDWGEHRCX

SEQ ID NO: 50 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
YCIWQDWGEHRCT

SEQ ID NO: 51 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 13: note = sarcosine
YCIWQEWGEHRCX

SEQ ID NO: 52 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 2-naphathalin
YCIXQDWGEHRCT

SEQ ID NO: 53 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
YCIWQDWGEHRCT

SEQ ID NO: 54 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  SITE 11: note = Lysine residue covalently linked to a lipophilic group via its side chain
  MOD_RES 13: note = sarcosine
YCIWQDWGEHKCX

SEQ ID NO: 55 - protein, 13 aa, synthetic construct
  REGION 1..13: note = Description of Artificial Sequence: Synthetic peptide
  REGION 1..13: note = 13-mer peptide portion of the compstatin analogue
  DISULFID 2..12: note = Intrachain disulfide bond
  MOD_RES 4: note = 1-methyl-tryptophan
  MOD_RES 8: note = sarcosine
  MOD_RES 13: note = sarcosine
YCIWQEWXEHRCX

SEQ ID NO: 56 - protein, 4 aa, synthetic construct
  REGION 1..4: note = Description of Artificial Sequence: Synthetic peptide
  REGION 1..4: note = N-terminal and/or C-terminal flanking sequence
ESSA

SEQ ID NO: 57 - protein, 4 aa, synthetic construct
  REGION 1..4: note = Description of Artificial Sequence: Synthetic peptide
  REGION 1..4: note = N-terminal and/or C-terminal flanking sequence
AKGE

SEQ ID NO: 58 - protein, 4 aa, synthetic construct
  REGION 1..4: note = Description of Artificial Sequence: Synthetic peptide
  REGION 1..4: note = N-terminal and/or C-terminal flanking sequence
ASSE

SEQ ID NO: 59 - protein, 4 aa, synthetic construct
  REGION 1..4: note = Description of Artificial Sequence: Synthetic peptide
  REGION 1..4: note = N-terminal and/or C-terminal flanking sequence
ASES

SEQ ID NO: 60 - protein, 4 aa, synthetic construct
  REGION 1..4: note = Description of Artificial Sequence: Synthetic peptide
  REGION 1..4: note = N-terminal and/or C-terminal flanking sequence
GSAE

SEQ ID NO: 61 - protein, 4 aa, synthetic construct
  REGION 1..4: note = Description of Artificial Sequence: Synthetic peptide
  REGION 1..4: note = N-terminal and/or C-terminal flanking sequence
ESSE

SEQ ID NO: 62 - protein, 4 aa, synthetic construct
  REGION 1..4: note = Description of Artificial Sequence: Synthetic peptide
  REGION 1..4: note = N-terminal and/or C-terminal flanking sequence
ESGA

SEQ ID NO: 63 - protein, 4 aa, synthetic construct
  REGION 1..4: note = Description of Artificial Sequence: Synthetic peptide
  REGION 1..4: note = N-terminal and/or C-terminal flanking sequence
EGSA

SEQ ID NO: 64 - protein, 4 aa, synthetic construct
  REGION 1..4: note = Description of Artificial Sequence: Synthetic peptide
  REGION 1..4: note = N-terminal and/or C-terminal flanking sequence
YLEA

SEQ ID NO: 65 - protein, 4 aa, synthetic construct
  REGION 1..4: note = Description of Artificial Sequence: Synthetic peptide
  REGION 1..4: note = N-terminal and/or C-terminal flanking sequence
GAES

SEQ ID NO: 66 - protein, 4 aa, synthetic construct
  REGION 1..4: note = Description of Artificial Sequence: Synthetic peptide
  REGION 1..4: note = N-terminal and/or C-terminal flanking sequence
EYGS

SEQ ID NO: 67 - protein, 4 aa, synthetic construct
  REGION 1..4: note = Description of Artificial Sequence: Synthetic peptide
  REGION 1..4: note = N-terminal and/or C-terminal flanking sequence
EGYA

SEQ ID NO: 68 - protein, 4 aa, synthetic construct
  REGION 1..4: note = Description of Artificial Sequence: Synthetic peptide
  REGION 1..4: note = N-terminal and/or C-terminal flanking sequence
EAGS

SEQ ID NO: 69 - protein, 4 aa, synthetic construct
  REGION 1..4: note = Description of Artificial Sequence: Synthetic peptide
  REGION 1..4: note = N-terminal and/or C-terminal flanking sequence
EAKS

SEQ ID NO: 70 - protein, 4 aa, synthetic construct
  REGION 1..4: note = Description of Artificial Sequence: Synthetic peptide
  REGION 1..4: note = N-terminal and/or C-terminal flanking sequence
EKSA

SEQ ID NO: 71 - protein, 4 aa, synthetic construct
  REGION 1..4: note = Description of Artificial Sequence: Synthetic peptide
  REGION 1..4: note = N-terminal and/or C-terminal flanking sequence
EGGS

SEQ ID NO: 72 - protein, 4 aa, synthetic construct
  REGION 1..4: note = Description of Artificial Sequence: Synthetic peptide
  REGION 1..4: note = N-terminal and/or C-terminal flanking sequence
EGGA

SEQ ID NO: 73 - protein, 4 aa, synthetic construct
  REGION 1..4: note = Description of Artificial Sequence: Synthetic peptide
  REGION 1..4: note = N-terminal and/or C-terminal flanking sequence
ESSG

SEQ ID NO: 74 - protein, 4 aa, synthetic construct
  REGION 1..4: note = Description of Artificial Sequence: Synthetic peptide
  REGION 1..4: note = N-terminal and/or C-terminal flanking sequence
ESAG

SEQ ID NO: 75 - protein, 4 aa, synthetic construct
  REGION 1..4: note = Description of Artificial Sequence: Synthetic peptide
  REGION 1..4: note = N-terminal and/or C-terminal flanking sequence
GEES

SEQ ID NO: 76 - protein, 4 aa, synthetic construct
  REGION 1..4: note = Description of Artificial Sequence: Synthetic peptide
  REGION 1..4: note = N-terminal and/or C-terminal flanking sequence
AEES

SEQ ID NO: 77 - protein, 4 aa, synthetic construct
  REGION 1..4: note = Description of Artificial Sequence: Synthetic peptide
  REGION 1..4: note = N-terminal and/or C-terminal flanking sequence
ESEG

SEQ ID NO: 78 - protein, 4 aa, synthetic construct
  REGION 1..4: note = Description of Artificial Sequence: Synthetic peptide
  REGION 1..4: note = N-terminal and/or C-terminal flanking sequence
AEGS

SEQ ID NO: 79 - protein, 4 aa, synthetic construct
  REGION 1..4: note = Description of Artificial Sequence: Synthetic peptide
  REGION 1..4: note = N-terminal and/or C-terminal flanking sequence
ESGS

SEQ ID NO: 80 - protein, 4 aa, synthetic construct
  REGION 1..4: note = Description of Artificial Sequence: Synthetic peptide
  REGION 1..4: note = N-terminal and/or C-terminal flanking sequence
SEGA

SEQ ID NO: 81 - protein, 4 aa, synthetic construct
  REGION 1..4: note = Description of Artificial Sequence: Synthetic peptide
  REGION 1..4: note = N-terminal and/or C-terminal flanking sequence
EGEA

SEQ ID NO: 82 - protein, 4 aa, synthetic construct
  REGION 1..4: note = Description of Artificial Sequence: Synthetic peptide
  REGION 1..4: note = N-terminal and/or C-terminal flanking sequence
EGSE

SEQ ID NO: 83 - protein, 4 aa, synthetic construct
  REGION 1..4: note = Description of Artificial Sequence: Synthetic peptide
  REGION 1..4: note = N-terminal and/or C-terminal flanking sequence
AGSE

SEQ ID NO: 84 - protein, 4 aa, synthetic construct
  REGION 1..4: note = Description of Artificial Sequence: Synthetic peptide
  REGION 1..4: note = N-terminal and/or C-terminal flanking sequence
SASE

SEQ ID NO: 85 - protein, 4 aa, synthetic construct
  REGION 1..4: note = Description of Artificial Sequence: Synthetic peptide
  REGION 1..4: note = N-terminal and/or C-terminal flanking sequence
EYSE

SEQ ID NO: 86 - protein, 4 aa, synthetic construct
  REGION 1..4: note = Description of Artificial Sequence: Synthetic peptide
  REGION 1..4: note = N-terminal and/or C-terminal flanking sequence
KGSA

SEQ ID NO: 87 - protein, 4 aa, synthetic construct
  REGION 1..4: note = Description of Artificial Sequence: Synthetic peptide
  REGION 1..4: note = N-terminal and/or C-terminal flanking sequence
ASGE

SEQ ID NO: 88 - protein, 7 aa, synthetic construct
  REGION 1..7: note = Description of Artificial Sequence: Synthetic peptide
  REGION 1..7: note = N-terminal and/or C-terminal flanking sequence
EGASGSG

SEQ ID NO: 89 - protein, 6 aa, synthetic construct
  REGION 1..6: note = Description of Artificial Sequence: Synthetic peptide
  REGION 1..6: note = N-terminal and/or C-terminal flanking sequence
EGAGSG

SEQ ID NO: 90 - protein, 6 aa, synthetic construct
  REGION 1..6: note = Description of Artificial Sequence: Synthetic peptide
  REGION 1..6: note = N-terminal and/or C-terminal flanking sequence
EGASAG

SEQ ID NO: 91 - protein, 6 aa, synthetic construct
  REGION 1..6: note = Description of Artificial Sequence: Synthetic peptide
  REGION 1..6: note = N-terminal and/or C-terminal flanking sequence
EGAGAG

SEQ ID NO: 92 - protein, 7 aa, synthetic construct
  REGION 1..7: note = Description of Artificial Sequence: Synthetic peptide
  REGION 1..7: note = N-terminal and/or C-terminal flanking sequence
EGESGSG

SEQ ID NO: 93 - protein, 6 aa, synthetic construct
  REGION 1..6: note = Description of Artificial Sequence: Synthetic peptide
  REGION 1..6: note = N-terminal and/or C-terminal flanking sequence
EGEGSG

SEQ ID NO: 94 - protein, 6 aa, synthetic construct
  REGION 1..6: note = Description of Artificial Sequence: Synthetic peptide
  REGION 1..6: note = N-terminal and/or C-terminal flanking sequence
EGESAG

SEQ ID NO: 95 - protein, 6 aa, synthetic construct
  REGION 1..6: note = Description of Artificial Sequence: Synthetic peptide
  REGION 1..6: note = N-terminal and/or C-terminal flanking sequence
EGEGAG

SEQ ID NO: 96 - protein, 5 aa, synthetic construct
  REGION 1..5: note = Description of Artificial Sequence: Synthetic peptide
  REGION 1..5: note = N-terminal and/or C-terminal flanking sequence
  SITE 3: note = glutamate participating in the peptide bond via the gamma-carboxylic acid
EKEAK

SEQ ID NO: 97 - protein, 5 aa, synthetic construct
  REGION 1..5: note = Description of Artificial Sequence: Synthetic peptide
  REGION 1..5: note = N-terminal and/or C-terminal flanking sequence
EGEGG

SEQ ID NO: 98 - protein, 5 aa, synthetic construct
  REGION 1..5: note = Description of Artificial Sequence: Synthetic peptide
  REGION 1..5: note = N-terminal and/or C-terminal flanking sequence
EGAGG

SEQ ID NO: 99 - protein, 5 aa, synthetic construct
  REGION 1..5: note = Description of Artificial Sequence: Synthetic peptide
  REGION 1..5: note = N-terminal and/or C-terminal flanking sequence
EGESS

SEQ ID NO: 100 - protein, 5 aa, synthetic construct
  REGION 1..5: note = Description of Artificial Sequence: Synthetic peptide
  REGION 1..5: note = N-terminal and/or C-terminal flanking sequence
GAESK

SEQ ID NO: 101 - protein, 4 aa, synthetic construct
  REGION 1..4: note = Description of Artificial Sequence: Synthetic peptide
  REGION 1..4: note = N-terminal and/or C-terminal flanking sequence
EGAK

SEQ ID NO: 102 - protein, 4 aa, synthetic construct
  REGION 1..4: note = Description of Artificial Sequence: Synthetic peptide
  REGION 1..4: note = N-terminal and/or C-terminal flanking sequence
EGEK

SEQ ID NO: 103 - protein, 4 aa, synthetic construct
  REGION 1..4: note = Description of Artificial Sequence: Synthetic peptide
  REGION 1..4: note = N-terminal and/or C-terminal flanking sequence
EGKK

SEQ ID NO: 104 - protein, 4 aa, synthetic construct
  REGION 1..4: note = Description of Artificial Sequence: Synthetic peptide
  REGION 1..4: note = N-terminal and/or C-terminal flanking sequence
  SITE 3: note = glutamate participating in the peptide bond via the gamma-carboxylic acid
EKEK

SEQ ID NO: 105-106 - skipped (2 entries)

SEQ ID NO: 107 - protein, 6 aa, synthetic construct
  REGION 1..6: note = Description of Artificial Sequence: Synthetic peptide
  REGION 1..6: note = N-terminal and/or C-terminal flanking sequence
GESESE

SEQ ID NO: 108 - protein, 6 aa, synthetic construct
  REGION 1..6: note = Description of Artificial Sequence: Synthetic peptide
  REGION 1..6: note = N-terminal and/or C-terminal flanking sequence
GAESES

SEQ ID NO: 109 - protein, 6 aa, synthetic construct
  REGION 1..6: note = Description of Artificial Sequence: Synthetic peptide
  REGION 1..6: note = N-terminal and/or C-terminal flanking sequence
EGESES

SEQ ID NO: 110 - protein, 7 aa, synthetic construct
  REGION 1..7: note = Description of Artificial Sequence: Synthetic peptide
  REGION 1..7: note = N-terminal and/or C-terminal flanking sequence
EGESESK

SEQ ID NO: 111-114 - skipped (4 entries)

SEQ ID NO: 115 - protein, 6 aa, synthetic construct
  REGION 1..6: note = Description of Artificial Sequence: Synthetic peptide
  REGION 1..6: note = N-terminal and/or C-terminal flanking sequence
EGEGGG

SEQ ID NO: 116 - protein, 7 aa, synthetic construct
  REGION 1..7: note = Description of Artificial Sequence: Synthetic peptide
  REGION 1..7: note = N-terminal and/or C-terminal flanking sequence
EGEGGGK

SEQ ID NO: 117 - protein, 6 aa, synthetic construct
  REGION 1..6: note = Description of Artificial Sequence: Synthetic peptide
  REGION 1..6: note = N-terminal and/or C-terminal flanking sequence
  SITE 3: note = glutamate participating in the peptide bond via the gamma-carboxylic acid
EKEGGG

SEQ ID NO: 118 - protein, 7 aa, synthetic construct
  REGION 1..7: note = Description of Artificial Sequence: Synthetic peptide
  REGION 1..7: note = N-terminal and/or C-terminal flanking sequence
  SITE 3: note = glutamate participating in the peptide bond via the gamma-carboxylic acid
EKEGGGK

SEQ ID NO: 119-120 - skipped (2 entries)

SEQ ID NO: 121 - protein, 5 aa, synthetic construct
  REGION 1..5: note = Description of Artificial Sequence: Synthetic peptide
  REGION 1..5: note = a sequence linked to the C terminus of the peptide
  SITE 3: note = glutamate participating in the peptide bond via the gamma-carboxylic acid
  SITE 5: note = Lysine residue covalently linked to a lipophilic group via its side chain
EKEAK

SEQ ID NO: 122 - protein, 4 aa, synthetic construct
  REGION 1..4: note = Description of Artificial Sequence: Synthetic peptide
  REGION 1..4: note = a sequence linked to the C terminus of the peptide
  SITE 4: note = Lysine residue covalently linked to a lipophilic group via its side chain
EGKK

SEQ ID NO: 123 - protein, 4 aa, synthetic construct
  REGION 1..4: note = Description of Artificial Sequence: Synthetic peptide
  REGION 1..4: note = a sequence linked to the C terminus of the peptide
  SITE 3: note = glutamate participating in the peptide bond via the gamma-carboxylic acid
  SITE 4: note = Lysine residue covalently linked to a lipophilic group via its side chain
EKEK

SEQ ID NO: 124 - skipped

SEQ ID NO: 125 - protein, 7 aa, synthetic construct
  REGION 1..7: note = Description of Artificial Sequence: Synthetic peptide
  REGION 1..7: note = a sequence linked to the C terminus of the peptide
  SITE 7: note = Lysine residue covalently linked to a lipophilic group via its side chain
EGESESK

SEQ ID NO: 126-128 - skipped (3 entries)

SEQ ID NO: 129 - protein, 7 aa, synthetic construct
  REGION 1..7: note = Description of Artificial Sequence: Synthetic peptide
  REGION 1..7: note = a sequence linked to the C terminus of the peptide
  SITE 7: note = Lysine residue covalently linked to a lipophilic group via its side chain
EGEGGGK

SEQ ID NO: 130 - protein, 7 aa, synthetic construct
  REGION 1..7: note = Description of Artificial Sequence: Synthetic peptide
  REGION 1..7: note = a sequence linked to the C terminus of the peptide
  SITE 3: note = glutamate participating in the peptide bond via the gamma-carboxylic acid
  SITE 7: note = Lysine residue covalently linked to a lipophilic group via its side chain
EKEGGGK

SEQ ID NO: 131 - skipped

SEQ ID NO: 132 - protein, 5 aa, synthetic construct
  REGION 1..5: note = Description of Artificial Sequence: Synthetic peptide
  REGION 1..5: note = a sequence linked to the C terminus of the peptide
  SITE 5: note = Lysine residue covalently linked to a lipophilic group via its side chain
GAESK

SEQ ID NO: 133 - protein, 4 aa, synthetic construct
  REGION 1..4: note = Description of Artificial Sequence: Synthetic peptide
  REGION 1..4: note = a sequence linked to the C terminus of the peptide
  SITE 4: note = Lysine residue covalently linked to a lipophilic group via its side chain
EGAK

SEQ ID NO: 134 - protein, 4 aa, synthetic construct
  REGION 1..4: note = Description of Artificial Sequence: Synthetic peptide
  REGION 1..4: note = a sequence linked to the C terminus of the peptide
  SITE 4: note = Lysine residue covalently linked to a lipophilic group via its side chain
EGEK

SEQ ID NO: 135 - protein, 6 aa, synthetic construct
  REGION 1..6: note = Description of Artificial Sequence: Synthetic peptide
  REGION 1..6: note = a sequence linked to the C terminus of the peptide
  SITE 3: note = Lysine residue covalently linked to a lipophilic group via its side chain
EGKEGE

SEQ ID NO: 136 - protein, 7 aa, synthetic construct
  REGION 1..7: note = Description of Artificial Sequence: Synthetic peptide
  REGION 1..7: note = a sequence linked to the C terminus of the peptide
  SITE 7: note = Lysine residue covalently linked to a lipophilic group via its side chain
GESESEK

SEQ ID NO: 137 - skipped

SEQ ID NO: 138 - protein, 27 aa, synthetic construct
  REGION 1..27: note = Description of Artificial Sequence: Synthetic peptide
  REGION 1..27: note = full length compstatin
  DISULFID 2..12: note = Intrachain disulfide bond
ICVVQDWGHHRCTAGHMANLTSHASAI

SEQ ID NO: 139 - protein, 4 aa, synthetic construct
  REGION 1..4: note = Description of Artificial Sequence: Synthetic peptide
  REGION 1..4: note = spacer
  SITE 1: note = glutamate participating in the peptide bond via the gamma-carboxylic acid
  SITE 4: note = glutamate participating in the peptide bond via the gamma-carboxylic acid
EKGE

SEQ ID NO: 140 - protein, 17 aa, synthetic construct
  REGION 1..17: note = Description of Artificial Sequence: Synthetic peptide
  REGION 1..17: note = compstatin analogue
  DISULFID 6..16: note = Intrachain disulfide bond
ESSAICIWQDWGEHRCT

SEQ ID NO: 141 - protein, 13 aa, synthetic construct
  REGION 1..13: note = Description of Artificial Sequence: Synthetic peptide
  REGION 1..13: note = compstatin analogue
  DISULFID 2..12: note = Intrachain disulfide bond
  MOD_RES 4: note = 1-methyl-tryptophan
ICIWQDWGEHRCT

SEQ ID NO: 142 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
ICIWQKWGAHRCTGAES

SEQ ID NO: 143 - protein, 17 aa, synthetic construct
  REGION 1..17: note = Description of Artificial Sequence: Synthetic peptide
  REGION 1..17: note = compstatin analogue
  DISULFID 6..16: note = Intrachain disulfide bond
ESSAYCIWQDWGEHRCT

SEQ ID NO: 144 - protein, 21 aa, synthetic construct
  REGION 1..21: note = Description of Artificial Sequence: Synthetic peptide
  REGION 1..21: note = compstatin analogue
  DISULFID 6..16: note = Intrachain disulfide bond
ESSAICIWQDWGEHRCTGAES

SEQ ID NO: 145 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
ICIWQDWGEHRCTGAES

SEQ ID NO: 146 - protein, 16 aa, synthetic construct
  REGION 1..16: note = Description of Artificial Sequence: Synthetic peptide
  REGION 1..16: note = compstatin analogue
  DISULFID 2..12: note = Intrachain disulfide bond
ICIWQDWGEHRCTEGE

SEQ ID NO: 147 - protein, 15 aa, synthetic construct
  REGION 1..15: note = Description of Artificial Sequence: Synthetic peptide
  REGION 1..15: note = compstatin analogue
  DISULFID 2..12: note = Intrachain disulfide bond
ICIWQDWGEHRCTEA

SEQ ID NO: 148 - protein, 14 aa, synthetic construct
  REGION 1..14: note = Description of Artificial Sequence: Synthetic peptide
  REGION 1..14: note = compstatin analogue
  DISULFID 2..12: note = Intrachain disulfide bond
ICIWQDWGEHRCTE

SEQ ID NO: 149 - protein, 15 aa, synthetic construct
  REGION 1..15: note = Description of Artificial Sequence: Synthetic peptide
  REGION 1..15: note = compstatin analogue
  DISULFID 2..12: note = Intrachain disulfide bond
ICIWQDWGEHRCEGE

SEQ ID NO: 150 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 17: note = sarcosine
EGSAICIWQDWGEHRCXE

SEQ ID NO: 151 - protein, 17 aa, synthetic construct
  REGION 1..17: note = Description of Artificial Sequence: Synthetic peptide
  REGION 1..17: note = compstatin analogue
  DISULFID 6..16: note = Intrachain disulfide bond
EGSAICIWQDWGEHRCT

SEQ ID NO: 152 - protein, 16 aa, synthetic construct
  REGION 1..16: note = Description of Artificial Sequence: Synthetic peptide
  REGION 1..16: note = compstatin analogue
  DISULFID 5..15: note = Intrachain disulfide bond
EGEICIWQDWGEHRCT

SEQ ID NO: 153 - protein, 16 aa, synthetic construct
  REGION 1..16: note = Description of Artificial Sequence: Synthetic peptide
  REGION 1..16: note = compstatin analogue
  DISULFID 5..15: note = Intrachain disulfide bond
ESEICIWQDWGEHRCT

SEQ ID NO: 154 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
SEICIWQDWGEHRCTEA

SEQ ID NO: 155 - protein, 15 aa, synthetic construct
  REGION 1..15: note = Description of Artificial Sequence: Synthetic peptide
  REGION 1..15: note = compstatin analogue
  DISULFID 3..13: note = Intrachain disulfide bond
EICIWQDWGEHRCTE

SEQ ID NO: 156 - protein, 17 aa, synthetic construct
  REGION 1..17: note = Description of Artificial Sequence: Synthetic peptide
  REGION 1..17: note = compstatin analogue
  DISULFID 3..13: note = Intrachain disulfide bond
EICIWQDWGEHRCTEGE

SEQ ID NO: 157 - protein, 18 aa, synthetic construct
  REGION 1..18: note = Description of Artificial Sequence: Synthetic peptide
  REGION 1..18: note = compstatin analogue
  DISULFID 5..15: note = Intrachain disulfide bond
EGEICIWQDWGEHRCEGE

SEQ ID NO: 158 - protein, 18 aa, synthetic construct
  REGION 1..18: note = Description of Artificial Sequence: Synthetic peptide
  REGION 1..18: note = compstatin analogue
  DISULFID 5..15: note = Intrachain disulfide bond
ESEICIWQDWGEHRCEGE

SEQ ID NO: 159 - protein, 19 aa, synthetic construct
  REGION 1..19: note = Description of Artificial Sequence: Synthetic peptide
  REGION 1..19: note = compstatin analogue
  DISULFID 5..15: note = Intrachain disulfide bond
KEKICIWQDWGEHRCTEKE

SEQ ID NO: 160 - protein, 19 aa, synthetic construct
  REGION 1..19: note = Description of Artificial Sequence: Synthetic peptide
  REGION 1..19: note = compstatin analogue
  DISULFID 5..15: note = Intrachain disulfide bond
EKGICIWQDWGEHRCTEKP

SEQ ID NO: 161 - protein, 16 aa, synthetic construct
  REGION 1..16: note = Description of Artificial Sequence: Synthetic peptide
  REGION 1..16: note = compstatin analogue
  DISULFID 2..12: note = Intrachain disulfide bond
ICIWQDWGEHRCTEGK

SEQ ID NO: 162 - protein, 17 aa, synthetic construct
  REGION 1..17: note = Description of Artificial Sequence: Synthetic peptide
  REGION 1..17: note = compstatin analogue
  DISULFID 5..15: note = Intrachain disulfide bond
  MOD_RES 16: note = sarcosine
GSAICIWQDWGEHRCXE

SEQ ID NO: 163 - protein, 16 aa, synthetic construct
  REGION 1..16: note = Description of Artificial Sequence: Synthetic peptide
  REGION 1..16: note = compstatin analogue
  DISULFID 4..14: note = Intrachain disulfide bond
  MOD_RES 15: note = sarcosine
SAICIWQDWGEHRCXE

SEQ ID NO: 164 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
SAICIWQDWGEHRCTEG

SEQ ID NO: 165 - protein, 16 aa, synthetic construct
  REGION 1..16: note = Description of Artificial Sequence: Synthetic peptide
  REGION 1..16: note = compstatin analogue
  DISULFID 2..12: note = Intrachain disulfide bond
FCIWQDWGEHRCTGAE

SEQ ID NO: 166 - protein, 20 aa, synthetic construct
  REGION 1..20: note = Description of Artificial Sequence: Synthetic peptide
  REGION 1..20: note = compstatin analogue
  DISULFID 6..16: note = Intrachain disulfide bond
  MOD_RES 17: note = sarcosine
EGSAICIWQDWGEHRCXEGE

SEQ ID NO: 167 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 17: note = sarcosine
EGSAFCIWQDWGEHRCXE

SEQ ID NO: 168 - protein, 17 aa, synthetic construct
  REGION 1..17: note = Description of Artificial Sequence: Synthetic peptide
  REGION 1..17: note = compstatin analogue
  DISULFID 6..16: note = Intrachain disulfide bond
ESSAICIWQDWGAHRCT

SEQ ID NO: 169 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
ICIWQDWGAHRCTGAES

SEQ ID NO: 170 - protein, 14 aa, synthetic construct
  REGION 1..14: note = Description of Artificial Sequence: Synthetic peptide
  REGION 1..14: note = compstatin analogue
  SITE 1: note = D-Tyrosine
  DISULFID 3..13: note = Intrachain disulfide bond
  MOD_RES 5: note = 1-methyl-tryptophan
  MOD_RES 9: note = sarcosine
  MOD_RES 14: note = N-methyl-isoleucine
YICIWQDWXAHRCI

SEQ ID NO: 171 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
  MOD_RES 17: note = sarcosine
EGSAICIWQDWGEHRCXE

SEQ ID NO: 172 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 2-naphathalin
  MOD_RES 17: note = sarcosine
EGSAICIXQDWGEHRCXE

SEQ ID NO: 173 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 1-methyl-tryptophan
ICIWQDWGEHRCTGAES

SEQ ID NO: 174 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 2-naphathalin
ICIXQDWGEHRCTGAES

SEQ ID NO: 175 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
  MOD_RES 17: note = sarcosine
EGSAFCIWQDWGEHRCXE

SEQ ID NO: 176 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
  MOD_RES 17: note = sarcosine
EGSAYCIWQDWGEHRCXE

SEQ ID NO: 177 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
EGSAICIWQDWGEHRCTE

SEQ ID NO: 178 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-naphathalin
EGSAFCIXQDWGEHRCTE

SEQ ID NO: 179 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
EGSAFCIWQDWGEHRCTE

SEQ ID NO: 180 - protein, 19 aa, synthetic construct
  REGION 1..19: note = Description of Artificial Sequence: Synthetic peptide
  REGION 1..19: note = compstatin analogue
  DISULFID 6..16: note = Intrachain disulfide bond
  MOD_RES 8: note = 1-methyl-tryptophan
EGSAFCIWQDWGEHRCEGE

SEQ ID NO: 181 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
EGSAYCIWQDWGEHRCTE

SEQ ID NO: 182 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 2-naphathalin
EGSAFCIXQDWGEHRCTE

SEQ ID NO: 183 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 1-methyl-tryptophan
FCIWQDWGEHRCTGAES

SEQ ID NO: 184 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 1-methyl-tryptophan
YCIWQDWGEHRCTGAES

SEQ ID NO: 185 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 1-naphathalin
FCIXQDWGEHRCTGAES

SEQ ID NO: 186 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 2-naphathalin
FCIXQDWGEHRCTGAES

SEQ ID NO: 187 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 2-naphathalin
YCIXQDWGEHRCTGAES

SEQ ID NO: 188 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
YCIWQDWGEHRCTGAES

SEQ ID NO: 189 - protein, 19 aa, synthetic construct
  REGION 1..19: note = Description of Artificial Sequence: Synthetic peptide
  REGION 1..19: note = compstatin analogue
  DISULFID 4..14: note = Intrachain disulfide bond
  MOD_RES 6: note = 1-methyl-tryptophan
SEFCIWQDWGEHRCTGAES

SEQ ID NO: 190 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 1-methyl-tryptophan
YCIWQDWGEHRCTEAGS

SEQ ID NO: 191 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 1-methyl-tryptophan
YCIWQDWGEHRCTESGA

SEQ ID NO: 192 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
  MOD_RES 17: note = sarcosine
EGSAYCIWQEWGEHRCXE

SEQ ID NO: 193 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 15: note = sarcosine
SEYCIWQDWGEHRCXEA

SEQ ID NO: 194 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  MOD_RES 4: note = 1-methyl-tryptophan
  MOD_RES 8: note = sarcosine
FCIWQDWXEHRCTGAES

SEQ ID NO: 195 - protein, 18 aa, synthetic construct
  REGION 1..18: note = Description of Artificial Sequence: Synthetic peptide
  REGION 1..18: note = compstatin analogue
  SITE 1: note = D-Tyrosine
  DISULFID 3..13: note = Intrachain disulfide bond
  MOD_RES 5: note = 1-methyl-tryptophan
  MOD_RES 9: note = sarcosine
YFCIWQDWXEHRCTGAES

SEQ ID NO: 196 - protein, 19 aa, synthetic construct
  REGION 1..19: note = Description of Artificial Sequence: Synthetic peptide
  REGION 1..19: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXGAES

SEQ ID NO: 197 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXEA

SEQ ID NO: 198 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 10: note = sarcosine
  MOD_RES 15: note = sarcosine
SEFCIWQDWXEHRCXEA

SEQ ID NO: 199 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 10: note = sarcosine
SEFCIWQDWXEHRCTEA

SEQ ID NO: 200 - protein, 16 aa, synthetic construct
  REGION 1..16: note = Description of Artificial Sequence: Synthetic peptide
  REGION 1..16: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXE

SEQ ID NO: 201 - protein, 16 aa, synthetic construct
  REGION 1..16: note = Description of Artificial Sequence: Synthetic peptide
  REGION 1..16: note = compstatin analogue
  DISULFID 4..14: note = Intrachain disulfide bond
  MOD_RES 6: note = 1-methyl-tryptophan
  MOD_RES 10: note = sarcosine
  MOD_RES 15: note = sarcosine
SEFCIWQDWXEHRCXE

SEQ ID NO: 202 - protein, 16 aa, synthetic construct
  REGION 1..16: note = Description of Artificial Sequence: Synthetic peptide
  REGION 1..16: note = compstatin analogue
  DISULFID 3..13: note = Intrachain disulfide bond
  MOD_RES 5: note = 1-methyl-tryptophan
  MOD_RES 14: note = sarcosine
EFCIWQDWGEHRCXEA

SEQ ID NO: 203 - protein, 17 aa, synthetic construct
  REGION 1..17: note = Description of Artificial Sequence: Synthetic peptide
  REGION 1..17: note = compstatin analogue
  MOD_RES 3: note = sarcosine
  DISULFID 4..14: note = Intrachain disulfide bond
  MOD_RES 6: note = 1-methyl-tryptophan
  MOD_RES 15: note = sarcosine
SEXCIWQDWGEHRCXEA

SEQ ID NO: 204 - protein, 17 aa, synthetic construct
  REGION 1..17: note = Description of Artificial Sequence: Synthetic peptide
  REGION 1..17: note = compstatin analogue
  MOD_RES 3: note = sarcosine
  DISULFID 4..14: note = Intrachain disulfide bond
  MOD_RES 6: note = 1-methyl-tryptophan
SEXCIWQDWGEHRCTEA

SEQ ID NO: 205 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 15: note = sarcosine
SEFCIWQEWGEHRCXEA

SEQ ID NO: 206 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
SEFCIWQDWGEHRCSEA

SEQ ID NO: 207 - protein, 15 aa, synthetic construct
  REGION 1..15: note = Description of Artificial Sequence: Synthetic peptide
  REGION 1..15: note = compstatin analogue
  DISULFID 3..13: note = Intrachain disulfide bond
  MOD_RES 5: note = 1-methyl-tryptophan
EFCIWQDWGEHRCES

SEQ ID NO: 208 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHKCXEA

SEQ ID NO: 209 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 15: note = sarcosine
GEFCIWQDWGEHRCXEA

SEQ ID NO: 210 - protein, 17 aa, synthetic construct
  REGION 1..17: note = Description of Artificial Sequence: Synthetic peptide
  REGION 1..17: note = compstatin analogue
  MOD_RES 3: note = sarcosine
  DISULFID 4..14: note = Intrachain disulfide bond
  MOD_RES 6: note = 1-methyl-tryptophan
GEXCIWQDWGEHRCTEA

SEQ ID NO: 211 - protein, 17 aa, synthetic construct
  REGION 1..17: note = Description of Artificial Sequence: Synthetic peptide
  REGION 1..17: note = compstatin analogue
  MOD_RES 3: note = sarcosine
  DISULFID 4..14: note = Intrachain disulfide bond
  MOD_RES 6: note = 1-methyl-tryptophan
  MOD_RES 10: note = sarcosine
SEXCIWQEWXEHRCTEA

SEQ ID NO: 212 - protein, 17 aa, synthetic construct
  REGION 1..17: note = Description of Artificial Sequence: Synthetic peptide
  REGION 1..17: note = compstatin analogue
  MOD_RES 3: note = sarcosine
  DISULFID 4..14: note = Intrachain disulfide bond
  MOD_RES 6: note = 1-methyl-tryptophan
  MOD_RES 15: note = sarcosine
SEXCIWQEWGEHRCXEA

SEQ ID NO: 213 - protein, 16 aa, synthetic construct
  REGION 1..16: note = Description of Artificial Sequence: Synthetic peptide
  REGION 1..16: note = compstatin analogue
  SITE 1: note = D-Tyrosine
  MOD_RES 2: note = sarcosine
  DISULFID 3..13: note = Intrachain disulfide bond
  MOD_RES 5: note = 1-methyl-tryptophan
YXCIWQDWGEHRCTEA

SEQ ID NO: 214 - protein, 20 aa, synthetic construct
  REGION 1..20: note = Description of Artificial Sequence: Synthetic peptide
  REGION 1..20: note = compstatin analogue
  SITE 1: note = Lysine residue covalently linked to a lipophilic group via its side chain
  DISULFID 6..16: note = Intrachain disulfide bond
KGSAICIWQDWGEHRCTEGE

SEQ ID NO: 215 - protein, 21 aa, synthetic construct
  REGION 1..21: note = Description of Artificial Sequence: Synthetic peptide
  REGION 1..21: note = compstatin analogue
  DISULFID 6..16: note = Intrachain disulfide bond
  MOD_RES 8: note = 1-methyl-tryptophan
  MOD_RES 17: note = sarcosine
  SITE 21: note = Lysine residue covalently linked to a lipophilic group via its side chain
ASGEYCIWQDWGEHRCXEGEK

SEQ ID NO: 216 - protein, 17 aa, synthetic construct
  REGION 1..17: note = Description of Artificial Sequence: Synthetic peptide
  REGION 1..17: note = compstatin analogue
  DISULFID 3..13: note = Intrachain disulfide bond
  MOD_RES 5: note = 1-methyl-tryptophan
  SITE 17: note = Lysine residue covalently linked to a lipophilic group via its side chain
EFCIWQDWGEHRCEGEK

SEQ ID NO: 217 - protein, 20 aa, synthetic construct
  REGION 1..20: note = Description of Artificial Sequence: Synthetic peptide
  REGION 1..20: note = compstatin analogue
  DISULFID 6..16: note = Intrachain disulfide bond
  SITE 20: note = Lysine residue covalently linked to a lipophilic group via its side chain
EGSAICIWQDWGEHRCTEGK

SEQ ID NO: 218 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
  SITE 15: note = Lysine residue covalently linked to a lipophilic group via its side chain
  MOD_RES 17: note = sarcosine
EGSAYCIWQDWGEHKCXE

SEQ ID NO: 219 - protein, 20 aa, synthetic construct
  REGION 1..20: note = Description of Artificial Sequence: Synthetic peptide
  REGION 1..20: note = compstatin analogue
  DISULFID 6..16: note = Intrachain disulfide bond
  MOD_RES 8: note = 1-methyl-tryptophan
  MOD_RES 17: note = sarcosine
  SITE 20: note = Lysine residue covalently linked to a lipophilic group via its side chain
EGSAYCIWQDWGEHRCXEGK

SEQ ID NO: 220 - protein, 21 aa, synthetic construct
  REGION 1..21: note = Description of Artificial Sequence: Synthetic peptide
  REGION 1..21: note = compstatin analogue
  DISULFID 6..16: note = Intrachain disulfide bond
  MOD_RES 8: note = 1-methyl-tryptophan
  MOD_RES 17: note = sarcosine
  SITE 21: note = Lysine residue covalently linked to a lipophilic group via its side chain
EGSAYCIWQDWGEHRCXEGEK

SEQ ID NO: 221 - protein, 21 aa, synthetic construct
  REGION 1..21: note = Description of Artificial Sequence: Synthetic peptide
  REGION 1..21: note = compstatin analogue
  DISULFID 6..16: note = Intrachain disulfide bond
  MOD_RES 8: note = 1-methyl-tryptophan
  MOD_RES 17: note = sarcosine
  SITE 21: note = Lysine residue covalently linked to a lipophilic group via its side chain
EGSAYCIWQDWGEHRCXEGKK

SEQ ID NO: 222 - protein, 21 aa, synthetic construct
  REGION 1..21: note = Description of Artificial Sequence: Synthetic peptide
  REGION 1..21: note = compstatin analogue
  DISULFID 6..16: note = Intrachain disulfide bond
  MOD_RES 8: note = 1-methyl-tryptophan
  MOD_RES 17: note = sarcosine
  SITE 20: note = glutamate participating in the peptide bond via the gamma-carboxylic acid
  SITE 21: note = Lysine residue covalently linked to a lipophilic group via its side chain
EGSAYCIWQDWGEHRCXEKEK

SEQ ID NO: 223 - protein, 18 aa, synthetic construct
  REGION 1..18: note = Description of Artificial Sequence: Synthetic peptide
  REGION 1..18: note = compstatin analogue
  DISULFID 2..12: note = Intrachain disulfide bond
  MOD_RES 4: note = 1-methyl-tryptophan
  SITE 18: note = Lysine residue covalently linked to a lipophilic group via its side chain
FCIWQDWGEHRCTGAESK

SEQ ID NO: 224 - protein, 16 aa, synthetic construct
  REGION 1..16: note = Description of Artificial Sequence: Synthetic peptide
  REGION 1..16: note = compstatin analogue
  DISULFID 2..12: note = Intrachain disulfide bond
  SITE 16: note = Lysine residue covalently linked to a lipophilic group via its side chain
ICIWQDWGEHRCTEGK

SEQ ID NO: 225 - protein, 17 aa, synthetic construct
  REGION 1..17: note = Description of Artificial Sequence: Synthetic peptide
  REGION 1..17: note = compstatin analogue
  DISULFID 2..12: note = Intrachain disulfide bond
  SITE 17: note = Lysine residue covalently linked to a lipophilic group via its side chain
ICIWQDWGEHRCTEGEK

SEQ ID NO: 226 - protein, 17 aa, synthetic construct
  REGION 1..17: note = Description of Artificial Sequence: Synthetic peptide
  REGION 1..17: note = compstatin analogue
  DISULFID 4..14: note = Intrachain disulfide bond
  MOD_RES 6: note = 1-methyl-tryptophan
  MOD_RES 15: note = sarcosine
  SITE 17: note = Lysine residue covalently linked to a lipophilic group via its side chain
SAYCIWQDWGEHRCXEK

SEQ ID NO: 227 - protein, 19 aa, synthetic construct
  REGION 1..19: note = Description of Artificial Sequence: Synthetic peptide
  REGION 1..19: note = compstatin analogue
  DISULFID 4..14: note = Intrachain disulfide bond
  MOD_RES 6: note = 1-methyl-tryptophan
  MOD_RES 15: note = sarcosine
  SITE 19: note = Lysine residue covalently linked to a lipophilic group via its side chain
SEFCIWQDWGEHRCXEGAK

SEQ ID NO: 228 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 229 - protein, 22 aa, synthetic construct
  REGION 1..22: note = Description of Artificial Sequence: Synthetic peptide
  REGION 1..22: note = compstatin analogue
  DISULFID 4..14: note = Intrachain disulfide bond
  MOD_RES 6: note = 1-methyl-tryptophan
  MOD_RES 15: note = sarcosine
  SITE 22: note = Lysine residue covalently linked to a lipophilic group via its side chain
SEFCIWQDWGEHRCXEGEGGGK

SEQ ID NO: 230 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 231 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 232 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 233 - protein, 22 aa, synthetic construct
  REGION 1..22: note = Description of Artificial Sequence: Synthetic peptide
  REGION 1..22: note = compstatin analogue
  DISULFID 4..14: note = Intrachain disulfide bond
  MOD_RES 6: note = 1-methyl-tryptophan
  MOD_RES 15: note = sarcosine
  SITE 22: note = Lysine residue covalently linked to a lipophilic group via its side chain
SEFCIWQDWGEHRCXEGESESK

SEQ ID NO: 234 - protein, 22 aa, synthetic construct
  REGION 1..22: note = Description of Artificial Sequence: Synthetic peptide
  REGION 1..22: note = compstatin analogue
  DISULFID 4..14: note = Intrachain disulfide bond
  MOD_RES 6: note = 1-methyl-tryptophan
  MOD_RES 15: note = sarcosine
  SITE 18: note = glutamate participating in the peptide bond via the gamma-carboxylic acid
  SITE 22: note = Lysine residue covalently linked to a lipophilic group via its side chain
SEFCIWQDWGEHRCXEKEGGGK

SEQ ID NO: 235 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
SEFCIWQDWGEHRCTEGE

SEQ ID NO: 236 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
SEFCIWQDWGEHRCTEGE

SEQ ID NO: 237 - protein, 22 aa, synthetic construct
  REGION 1..22: note = Description of Artificial Sequence: Synthetic peptide
  REGION 1..22: note = compstatin analogue
  DISULFID 4..14: note = Intrachain disulfide bond
  MOD_RES 6: note = 1-methyl-tryptophan
  SITE 22: note = Lysine residue covalently linked to a lipophilic group via its side chain
SEFCIWQDWGEHRCTEGEGGGK

SEQ ID NO: 238 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
SEFCIWQDWGEHRCTEGE

SEQ ID NO: 239 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
SEFCIWQDWGEHRCTEGE

SEQ ID NO: 240 - protein, 22 aa, synthetic construct
  REGION 1..22: note = Description of Artificial Sequence: Synthetic peptide
  REGION 1..22: note = compstatin analogue
  DISULFID 4..14: note = Intrachain disulfide bond
  MOD_RES 6: note = 1-methyl-tryptophan
  SITE 18: note = glutamate participating in the peptide bond via the gamma-carboxylic acid
  SITE 22: note = Lysine residue covalently linked to a lipophilic group via its side chain
SEFCIWQDWGEHRCTEKEGGGK

SEQ ID NO: 241 - protein, 20 aa, synthetic construct
  REGION 1..20: note = Description of Artificial Sequence: Synthetic peptide
  REGION 1..20: note = compstatin analogue
  DISULFID 4..14: note = Intrachain disulfide bond
  MOD_RES 6: note = 1-methyl-tryptophan
  SITE 20: note = Lysine residue covalently linked to a lipophilic group via its side chain
SEFCIWQDWGEHRCTGAESK

SEQ ID NO: 242 - protein, 19 aa, synthetic construct
  REGION 1..19: note = Description of Artificial Sequence: Synthetic peptide
  REGION 1..19: note = compstatin analogue
  DISULFID 4..14: note = Intrachain disulfide bond
  MOD_RES 6: note = 1-methyl-tryptophan
  MOD_RES 15: note = sarcosine
  SITE 19: note = Lysine residue covalently linked to a lipophilic group via its side chain
SEFCIWQEWGEHRCXEGAK

SEQ ID NO: 243 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEFCIWQEWGEHRCXEGE

SEQ ID NO: 244 - protein, 19 aa, synthetic construct
  REGION 1..19: note = Description of Artificial Sequence: Synthetic peptide
  REGION 1..19: note = compstatin analogue
  DISULFID 4..14: note = Intrachain disulfide bond
  MOD_RES 6: note = 1-methyl-tryptophan
  MOD_RES 15: note = sarcosine
  SITE 19: note = Lysine residue covalently linked to a lipophilic group via its side chain
SEYCIWQDWGEHRCXEGAK

SEQ ID NO: 245 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEYCIWQDWGEHRCXEGE

SEQ ID NO: 246 - protein, 19 aa, synthetic construct
  REGION 1..19: note = Description of Artificial Sequence: Synthetic peptide
  REGION 1..19: note = compstatin analogue
  DISULFID 4..14: note = Intrachain disulfide bond
  MOD_RES 6: note = 1-methyl-tryptophan
  MOD_RES 15: note = sarcosine
  SITE 19: note = Lysine residue covalently linked to a lipophilic group via its side chain
SEYCIWQDWGEHRCXEGEK

SEQ ID NO: 247 - protein, 20 aa, synthetic construct
  REGION 1..20: note = Description of Artificial Sequence: Synthetic peptide
  REGION 1..20: note = compstatin analogue
  DISULFID 4..14: note = Intrachain disulfide bond
  MOD_RES 6: note = 1-methyl-tryptophan
  MOD_RES 10: note = sarcosine
  MOD_RES 15: note = sarcosine
  SITE 18: note = glutamate participating in the peptide bond via the gamma-carboxylic acid
  SITE 20: note = Lysine residue covalently linked to a lipophilic group via its side chain
SEYCIWQEWXEHRCXEKEAK

SEQ ID NO: 248 - protein, 19 aa, synthetic construct
  REGION 1..19: note = Description of Artificial Sequence: Synthetic peptide
  REGION 1..19: note = compstatin analogue
  DISULFID 4..14: note = Intrachain disulfide bond
  MOD_RES 6: note = 1-methyl-tryptophan
  MOD_RES 15: note = sarcosine
  SITE 19: note = Lysine residue covalently linked to a lipophilic group via its side chain
SEYCIWQEWGEHRCXEGAK

SEQ ID NO: 249 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEYCIWQEWGEHRCXEGE

SEQ ID NO: 250 - protein, 18 aa, synthetic construct
  REGION 1..18: note = Description of Artificial Sequence: Synthetic peptide
  REGION 1..18: note = compstatin analogue
  DISULFID 6..16: note = Intrachain disulfide bond
  MOD_RES 8: note = 1-methyl-tryptophan
  MOD_RES 17: note = sarcosine
EGSEYCIWQDWGEHRCXE

SEQ ID NO: 251 - protein, 20 aa, synthetic construct
  REGION 1..20: note = Description of Artificial Sequence: Synthetic peptide
  REGION 1..20: note = compstatin analogue
  DISULFID 6..16: note = Intrachain disulfide bond
ESSAICIWQDWGEHRCTEGE

SEQ ID NO: 252 - protein, 18 aa, synthetic construct
  REGION 1..18: note = Description of Artificial Sequence: Synthetic peptide
  REGION 1..18: note = compstatin analogue
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 253 - protein, 16 aa, synthetic construct
  REGION 1..16: note = Description of Artificial Sequence: Synthetic peptide
  REGION 1..16: note = compstatin analogue
  DISULFID 4..14: note = Intrachain disulfide bond
  MOD_RES 6: note = 1-methyl-tryptophan
  MOD_RES 10: note = sarcosine
  MOD_RES 15: note = sarcosine
SEFCIWQDWXEHRCXE

SEQ ID NO: 254 - protein, 16 aa, synthetic construct
  REGION 1..16: note = Description of Artificial Sequence: Synthetic peptide
  REGION 1..16: note = compstatin analogue
  DISULFID 3..13: note = Intrachain disulfide bond
  MOD_RES 5: note = 1-methyl-tryptophan
  MOD_RES 14: note = sarcosine
EFCIWQDWGEHRCXEA

SEQ ID NO: 255 - protein, 13 aa, synthetic construct
  REGION 1..13: note = Description of Artificial Sequence: Synthetic peptide
  REGION 1..13: note = 4W9A*
  DISULFID 2..12: note = Intrachain disulfide bond
ICVWQDWGAHRCT

SEQ ID NO: 256 - protein, 14 aa, synthetic construct
  REGION 1..14: note = Description of Artificial Sequence: Synthetic peptide
  REGION 1..14: note = compound A
  SITE 1: note = D-Tyrosine
  DISULFID 3..13: note = Intrachain disulfide bond
  MOD_RES 5: note = 1-methyl-tryptophan
  MOD_RES 9: note = sarcosine
  MOD_RES 14: note = N-methyl-isoleucine
YICVWQDWXAHRCI

SEQ ID NO: 257 - protein, 13 aa, synthetic construct
  REGION 1..13: note = Description of Artificial Sequence: Synthetic peptide
  REGION 1..13: note = Compound A
  DISULFID 2..12: note = Intrachain disulfide bond
ICVWQDWGEHRCT

SEQ ID NO: 258 - protein, 13 aa, synthetic construct
  REGION 1..13: note = Description of Artificial Sequence: Synthetic peptide
  REGION 1..13: note = Compound B
  DISULFID 2..12: note = Intrachain disulfide bond
ICVWQDWGSHRCT

SEQ ID NO: 259 - protein, 17 aa, synthetic construct
  REGION 1..17: note = Description of Artificial Sequence: Synthetic peptide
  REGION 1..17: note = Compound C
  DISULFID 6..16: note = Intrachain disulfide bond
ESSAICVWQDWGEHRCT

SEQ ID NO: 260 - protein, 17 aa, synthetic construct
  REGION 1..17: note = Description of Artificial Sequence: Synthetic peptide
  REGION 1..17: note = Compound D
  DISULFID 2..12: note = Intrachain disulfide bond
ICVWQDWGEHRCTGAES

SEQ ID NO: 261 - protein, 13 aa, synthetic construct
  REGION 1..13: note = Description of Artificial Sequence: Synthetic peptide
  REGION 1..13: note = Compound E
  DISULFID 2..12: note = Intrachain disulfide bond
ICVWQDWGAHSCT

SEQ ID NO: 262 - protein, 13 aa, synthetic construct
  REGION 1..13: note = Description of Artificial Sequence: Synthetic peptide
  REGION 1..13: note = Compound F
  DISULFID 2..12: note = Intrachain disulfide bond
ICVWQDWGEHSCT

SEQ ID NO: 263 - protein, 13 aa, synthetic construct
  REGION 1..13: note = Description of Artificial Sequence: Synthetic peptide
  REGION 1..13: note = Compound G
  DISULFID 2..12: note = Intrachain disulfide bond
ICVWQDWGEHRCS

SEQ ID NO: 264 - protein, 18 aa, synthetic construct
  REGION 1..18: note = Description of Artificial Sequence: Synthetic peptide
  REGION 1..18: note = Compound H
  DISULFID 6..16: note = Intrachain disulfide bond
  MOD_RES 17: note = sarcosine
EGSAICVWQDWGEHRCXE

SEQ ID NO: 265 - protein, 16 aa, synthetic construct
  REGION 1..16: note = Description of Artificial Sequence: Synthetic peptide
  REGION 1..16: note = Compound J
  DISULFID 2..12: note = Intrachain disulfide bond
ICVWQDWGEHRCTEGE

SEQ ID NO: 266 - protein, 4 aa, synthetic construct
  REGION 1..4: note = Description of Artificial Sequence: Synthetic peptide
  REGION 1..4: note = N-terminal and/or C-terminal flanking sequence
  SITE 3: note = glutamate participating in the peptide bond via the gamma-carboxylic acid
EKEA

SEQ ID NO: 267 - protein, 4 aa, synthetic construct
  REGION 1..4: note = Description of Artificial Sequence: Synthetic peptide
  MOD_RES 1: note = 17-carboxy-heptadecanoyl-gammaGlu
  SITE 4: note = glutamate participating in the peptide bond via the gamma-carboxylic acid
EKGE

SEQ ID NO: 268 - protein, 20 aa, synthetic construct
  REGION 1..20: note = Description of Artificial Sequence: Synthetic peptide
  MOD_RES 1: note = 15-carboxy-pentadecanoyl-gammaGlu-Peg3-Peg3 attached to side chain of Lysine
  DISULFID 6..16: note = Intrachain disulfide bond
KGSAICIWQDWGEHRCTEGE

SEQ ID NO: 269 - protein, 21 aa, synthetic construct
  REGION 1..21: note = Description of Artificial Sequence: Synthetic peptide
  DISULFID 6..16: note = Intrachain disulfide bond
  MOD_RES 8: note = 1-methyl-tryptophan
  MOD_RES 17: note = sarcosine
  MOD_RES 21: note = 17-carboxy-heptadecanoyl-gammaGlu-Gly-gammaGlu attached to side chain of Lysine
ASGEYCIWQDWGEHRCXEGEK

SEQ ID NO: 270 - protein, 17 aa, synthetic construct
  REGION 1..17: note = Description of Artificial Sequence: Synthetic peptide
  DISULFID 3..13: note = Intrachain disulfide bond
  MOD_RES 5: note = 1-methyl-tryptophan
  MOD_RES 17: note = 17-carboxy-heptadecanoyl-gammaGlu-Gly-gammaGlu attached to side chain of Lysine
EFCIWQDWGEHRCEGEK

SEQ ID NO: 271 - protein, 20 aa, synthetic construct
  REGION 1..20: note = Description of Artificial Sequence: Synthetic peptide
  DISULFID 6..16: note = Intrachain disulfide bond
  MOD_RES 20: note = 15-carboxy-pentadecanoyl-gammaGlu attached to side chain of Lysine
EGSAICIWQDWGEHRCTEGK

SEQ ID NO: 272 - protein, 18 aa, synthetic construct
  REGION 1..18: note = Description of Artificial Sequence: Synthetic peptide
  DISULFID 6..16: note = Intrachain disulfide bond
  MOD_RES 8: note = 1-methyl-tryptophan
  MOD_RES 15: note = 15-carboxy-pentadecanoyl-gammaGlu-Peg3-Peg3 attached to side chain of Lysine
  MOD_RES 17: note = sarcosine
EGSAYCIWQDWGEHKCXE

SEQ ID NO: 273 - protein, 20 aa, synthetic construct
  REGION 1..20: note = Description of Artificial Sequence: Synthetic peptide
  DISULFID 6..16: note = Intrachain disulfide bond
  MOD_RES 8: note = 1-methyl-tryptophan
  MOD_RES 17: note = sarcosine
  MOD_RES 20: note = 15-carboxy-pentadecanoyl-gammaGlu-Peg3-Peg3 attached to side chain of Lysine
EGSAYCIWQDWGEHRCXEGK

SEQ ID NO: 274 - protein, 21 aa, synthetic construct
  REGION 1..21: note = Description of Artificial Sequence: Synthetic peptide
  DISULFID 6..16: note = Intrachain disulfide bond
  MOD_RES 8: note = 1-methyl-tryptophan
  MOD_RES 17: note = sarcosine
  MOD_RES 21: note = 17-carboxy-heptadecanoyl-gammaGlu-Gly-gammaGlu attached to side chain of Lysine
EGSAYCIWQDWGEHRCXEGEK

SEQ ID NO: 275 - protein, 21 aa, synthetic construct
  REGION 1..21: note = Description of Artificial Sequence: Synthetic peptide
  DISULFID 6..16: note = Intrachain disulfide bond
  MOD_RES 8: note = 1-methyl-tryptophan
  MOD_RES 17: note = sarcosine
  MOD_RES 21: note = 17-carboxy-heptadecanoyl-gammaGlu-Gly-gammaGlu attached to side chain of Lysine
EGSAYCIWQDWGEHRCXEGKK

SEQ ID NO: 276 - protein, 21 aa, synthetic construct
  REGION 1..21: note = Description of Artificial Sequence: Synthetic peptide
  DISULFID 6..16: note = Intrachain disulfide bond
  MOD_RES 8: note = 1-methyl-tryptophan
  MOD_RES 17: note = sarcosine
  SITE 20: note = glutamate participating in the peptide bond via the gamma-carboxylic acid
  MOD_RES 21: note = 17-carboxy-heptadecanoyl-gammaGlu-Peg3-Peg3 attached to side chain of Lysine
EGSAYCIWQDWGEHRCXEKEK

SEQ ID NO: 277 - protein, 18 aa, synthetic construct
  REGION 1..18: note = Description of Artificial Sequence: Synthetic peptide
  DISULFID 2..12: note = Intrachain disulfide bond
  MOD_RES 4: note = 1-methyl-tryptophan
  MOD_RES 18: note = 15-carboxy-pentadecanoyl-gammaGlu-Peg3-Peg3 attached to side chain of Lysine
FCIWQDWGEHRCTGAESK

SEQ ID NO: 278 - protein, 16 aa, synthetic construct
  REGION 1..16: note = Description of Artificial Sequence: Synthetic peptide
  DISULFID 2..12: note = Intrachain disulfide bond
  MOD_RES 16: note = 15-carboxy-pentadecanoyl-gammaGlu attached to side chain of Lysine
ICIWQDWGEHRCTEGK

SEQ ID NO: 279 - protein, 16 aa, synthetic construct
  REGION 1..16: note = Description of Artificial Sequence: Synthetic peptide
  DISULFID 2..12: note = Intrachain disulfide bond
  MOD_RES 16: note = 15-carboxy-pentadecanoyl-gammaGlu-Peg3-Peg3 attached to side chain of Lysine
ICIWQDWGEHRCTEGK

SEQ ID NO: 280 - protein, 16 aa, synthetic construct
  REGION 1..16: note = Description of Artificial Sequence: Synthetic peptide
  DISULFID 2..12: note = Intrachain disulfide bond
  MOD_RES 16: note = 15-carboxy-pentadecanoyl-(Piperazine-1-yl)-acetyl-P eg3-Peg3 attached to side chain of Lysine
ICIWQDWGEHRCTEGK

SEQ ID NO: 281 - protein, 16 aa, synthetic construct
  REGION 1..16: note = Description of Artificial Sequence: Synthetic peptide
  DISULFID 2..12: note = Intrachain disulfide bond
  MOD_RES 16: note = 17-carboxy-heptadecanoyl-gammaGlu-Peg3-Peg3 attached to side chain of Lysine
ICIWQDWGEHRCTEGK

SEQ ID NO: 282 - protein, 16 aa, synthetic construct
  REGION 1..16: note = Description of Artificial Sequence: Synthetic peptide
  DISULFID 2..12: note = Intrachain disulfide bond
  MOD_RES 16: note = 19-carboxy-nonadecanoyl-gammaGlu-Peg3-Peg3 attached to side chain of Lysine
ICIWQDWGEHRCTEGK

SEQ ID NO: 283 - protein, 17 aa, synthetic construct
  REGION 1..17: note = Description of Artificial Sequence: Synthetic peptide
  DISULFID 2..12: note = Intrachain disulfide bond
  MOD_RES 17: note = 15-carboxy-pentadecanoyl-gammaGlu-Peg3-Peg3 attached to side chain of Lysine
ICIWQDWGEHRCTEGEK

SEQ ID NO: 284 - protein, 17 aa, synthetic construct
  REGION 1..17: note = Description of Artificial Sequence: Synthetic peptide
  DISULFID 2..12: note = Intrachain disulfide bond
  MOD_RES 17: note = 17-carboxy-heptadecanoyl-gammaGlu-Peg3-Peg3 attached to side chain of Lysine
ICIWQDWGEHRCTEGEK

SEQ ID NO: 285 - protein, 17 aa, synthetic construct
  REGION 1..17: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 17: note = 17-carboxy-heptadecanoyl-gammaGlu-Lys-Gly-gammaGlu attached to side chain of Lysine
SAYCIWQDWGEHRCXEK

SEQ ID NO: 286 - protein, 17 aa, synthetic construct
  REGION 1..17: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 17: note = 17-carboxy-heptadecanoyl-gammaGlu-Gly-gammaGlu attached to side chain of Lysine
SAYCIWQDWGEHRCXEK

SEQ ID NO: 287 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Gly-Peg3-gammaGlu -Peg3 attached to side chain of Lysine
SEFCIWQDWGEHRCXEGAK

SEQ ID NO: 288 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 289 - protein, 22 aa, synthetic construct
  REGION 1..22: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 22: note = 17-carboxy-heptadecanoyl-gammaGlu-Gly-gammaGlu attached to side chain of Lysine
SEFCIWQDWGEHRCXEGEGGGK

SEQ ID NO: 290 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 291 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 292 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 293 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 294 - protein, 22 aa, synthetic construct
  REGION 1..22: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 22: note = 17-carboxy-heptadecanoyl-gammaGlu-Gly-gammaGlu attached to side chain of Lysine
SEFCIWQDWGEHRCXEGESESK

SEQ ID NO: 295 - protein, 22 aa, synthetic construct
  REGION 1..22: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 22: note = 17-carboxy-heptadecanoyl-gammaGlu attached to side chain of Lysine
SEFCIWQDWGEHRCXEGESESK

SEQ ID NO: 296 - protein, 22 aa, synthetic construct
  REGION 1..22: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  SITE 18: note = glutamate participating in the peptide bond via the gamma-carboxylic acid
  MOD_RES 22: note = 17-carboxy-heptadecanoyl-gammaGlu-Gly-gammaGlu attached to side chain of Lysine
SEFCIWQDWGEHRCXEKEGGGK

SEQ ID NO: 297 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
SEFCIWQDWGEHRCTEGE

SEQ ID NO: 298 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
SEFCIWQDWGEHRCTEGE

SEQ ID NO: 299 - protein, 22 aa, synthetic construct
  REGION 1..22: note = Description of Artificial Sequence: Synthetic peptide
  DISULFID 4..14: note = Intrachain disulfide bond
  MOD_RES 6: note = 1-methyl-tryptophan
  MOD_RES 22: note = 17-carboxy-heptadecanoyl-gammaGlu-Gly-gammaGlu attached to side chain of Lysine
SEFCIWQDWGEHRCTEGEGGGK

SEQ ID NO: 300 - protein, 22 aa, synthetic construct
  REGION 1..22: note = Description of Artificial Sequence: Synthetic peptide
  DISULFID 4..14: note = Intrachain disulfide bond
  MOD_RES 6: note = 1-methyl-tryptophan
  MOD_RES 22: note = 15-carboxy-pentadecanoyl-gammaGlu-Gly-gammaGlu attached to side chain of Lysine
SEFCIWQDWGEHRCTEGEGGGK

SEQ ID NO: 301 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
SEFCIWQDWGEHRCTEGE

SEQ ID NO: 302 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
SEFCIWQDWGEHRCTEGE

SEQ ID NO: 303 - protein, 22 aa, synthetic construct
  REGION 1..22: note = Description of Artificial Sequence: Synthetic peptide
  DISULFID 4..14: note = Intrachain disulfide bond
  MOD_RES 6: note = 1-methyl-tryptophan
  SITE 18: note = glutamate participating in the peptide bond via the gamma-carboxylic acid
  MOD_RES 22: note = 17-carboxy-heptadecanoyl-gammaGlu-Gly-gammaGlu attached to side chain of Lysine
SEFCIWQDWGEHRCTEKEGGGK

SEQ ID NO: 304 - protein, 20 aa, synthetic construct
  REGION 1..20: note = Description of Artificial Sequence: Synthetic peptide
  DISULFID 4..14: note = Intrachain disulfide bond
  MOD_RES 6: note = 1-methyl-tryptophan
  MOD_RES 20: note = 15-carboxy-hexadecanoyl-gammaGlu-Gly-gammaGlu attached to side chain of Lysine
SEFCIWQDWGEHRCTGAESK

SEQ ID NO: 305 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Gly-Peg3-gammaGlu -Peg3 attached to side chain of Lysine
SEFCIWQEWGEHRCXEGAK

SEQ ID NO: 306 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEFCIWQEWGEHRCXEGE

SEQ ID NO: 307 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Gly-gammaGlu attached to side chain of Lysine
SEYCIWQDWGEHRCXEGAK

SEQ ID NO: 308 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Gly-Peg3-gammaGlu -Peg3 attached to side chain of Lysine
SEYCIWQDWGEHRCXEGAK

SEQ ID NO: 309 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEYCIWQDWGEHRCXEGE

SEQ ID NO: 310 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Gly-gammaGlu attached to side chain of Lysine
SEYCIWQDWGEHRCXEGEK

SEQ ID NO: 311 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Gly-gammaGlu attached to side chain of Lysine
SEYCIWQDWGEHRCXEGKK

SEQ ID NO: 312 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Lys-gammaGlu attached to side chain of Lysine
SEYCIWQDWGEHRCXEGEK

SEQ ID NO: 313 - protein, 20 aa, synthetic construct
  REGION 1..20: note = Description of Artificial Sequence: Synthetic peptide
  DISULFID 4..14: note = Intrachain disulfide bond
  MOD_RES 6: note = 1-methyl-tryptophan
  MOD_RES 10: note = sarcosine
  MOD_RES 15: note = sarcosine
  SITE 18: note = glutamate participating in the peptide bond via the gamma-carboxylic acid
  MOD_RES 20: note = 17-carboxy-heptadecanoyl-gammaGlu-Gly-Peg3-gammaGlu -Peg3 attached to side chain of Lysine
SEYCIWQEWXEHRCXEKEAK

SEQ ID NO: 314 - protein, 19 aa, synthetic construct
  REGION 1..19: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
  MOD_RES 19: note = 17-carboxy-heptadecanoyl-gammaGlu-Gly-Peg3-gammaGlu -Peg3 attached to side chain of Lysine
SEYCIWQEWGEHRCXEGAK

SEQ ID NO: 315 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEYCIWQEWGEHRCXEGE

SEQ ID NO: 316 - protein, 18 aa, synthetic construct
  REGION 1..18: note = Description of Artificial Sequence: Synthetic peptide
  MOD_RES 1: note = 15-Carboxy-pentadecanoyl attached to side chain of Glu
  DISULFID 6..16: note = Intrachain disulfide bond
  MOD_RES 8: note = 1-methyl-tryptophan
  MOD_RES 17: note = sarcosine
EGSEYCIWQDWGEHRCXE

SEQ ID NO: 317 - protein, 18 aa, synthetic construct
  REGION 1..18: note = Description of Artificial Sequence: Synthetic peptide
  MOD_RES 1: note = 17-Carboxy-heptadecanoyl attached to side chain of Glu
  DISULFID 6..16: note = Intrachain disulfide bond
  MOD_RES 8: note = 1-methyl-tryptophan
  MOD_RES 17: note = sarcosine
EGSEYCIWQDWGEHRCXE

SEQ ID NO: 318 - protein, 20 aa, synthetic construct
  REGION 1..20: note = Description of Artificial Sequence: Synthetic peptide
  MOD_RES 1: note = 15-Carboxy-pentadecanoyl attached to side chain of Glu
  DISULFID 6..16: note = Intrachain disulfide bond
ESSAICIWQDWGEHRCTEGE

SEQ ID NO: 319 - protein, 18 aa, synthetic construct
  REGION 1..18: note = Description of Artificial Sequence: Synthetic peptide
  DISULFID 4..14: note = Intrachain disulfide bond
  MOD_RES 6: note = 1-methyl-tryptophan
  MOD_RES 15: note = sarcosine
SEFCIWQDWGEHRCXEGE

SEQ ID NO: 320 - protein, 16 aa, synthetic construct
  REGION 1..16: note = Description of Artificial Sequence: Synthetic peptide
  DISULFID 4..14: note = Intrachain disulfide bond
  MOD_RES 6: note = 1-methyl-tryptophan
  MOD_RES 10: note = sarcosine
  MOD_RES 15: note = sarcosine
SEFCIWQDWXEHRCXE

SEQ ID NO: 321 - protein, 16 aa, synthetic construct
  REGION 1..16: note = Description of Artificial Sequence: Synthetic peptide
  DISULFID 3..13: note = Intrachain disulfide bond
  MOD_RES 5: note = 1-methyl-tryptophan
  MOD_RES 14: note = sarcosine
EFCIWQDWGEHRCXEA

SEQ ID NO: 322 - protein, 13 aa, synthetic construct
  REGION 1..13: note = Description of Artificial Sequence: Synthetic peptide
  DISULFID 2..12: note = Intrachain disulfide bond
ICIWQDWGAHRCT

SEQ ID NO: 323 - protein, 13 aa, synthetic construct
  REGION 1..13: note = Description of Artificial Sequence: Synthetic peptide
  DISULFID 2..12: note = Intrachain disulfide bond
ICVWQDWGAHRCT

SEQ ID NO: 324 - protein, 13 aa, synthetic construct
  REGION 1..13: note = Description of Artificial Sequence: Synthetic peptide
  DISULFID 2..12: note = Intrachain disulfide bond
  MOD_RES 3: note = Any amino acid
ICXWQDWGEHRCT

SEQ ID NO: 325 - protein, 4 aa, synthetic construct
  REGION 1..4: note = Description of Artificial Sequence: Synthetic peptide
  SITE 1: note = Lysine residue covalently linked to a lipophilic group via its side chain
KGSA

SEQ ID NO: 326 - protein, 14 aa, synthetic construct
  REGION 1..14: note = Description of Artificial Sequence: Synthetic peptide
  SITE 1: note = D-Tyrosine
  DISULFID 3..13: note = Intrachain disulfide bond
  MOD_RES 5: note = 1-methyl-tryptophan
  MOD_RES 9: note = sarcosine
  MOD_RES 14: note = N-methyl-isoleucine
YICVWQDWXAHRCI

SEQ ID NO: 327 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, F, V, Y, 1-Me-Trp, D-Trp, N-Me-Trp, 1-For-Trp, 1-Nal, 2-Nal, 5-Me-Trp, Bpa or 2-Igl
  VARIANT 12: note = E, K or D
  VARIANT 14: note = G or Sar
  VARIANT 17: note = R, S or K
  VARIANT 19: note = T, S, E, F, H, K, Sar, G, I, D, N-Me-Ile or N-Me-Thr
  VARIANT 20..27: note = A, E, G, L, K, F, P, S, T, W, Y, R, V, Sar, epsilonLys, gammaGlu, betaAsp, or betaAla, or a corresponding D form thereof or Peg3, Peg4, 8-aminooctanoyl derivatives thereof or may be absent
  VARIANT 20..27: note = This region may encompass 1 to 8 amino acid residues
XXXXXXXCIXQXWXEHXCXXXXXXXXX

SEQ ID NO: 328 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 2-Nal, 1-Nal or 1-Me-Trp
  VARIANT 12: note = E or D
  VARIANT 14: note = G or Sar
  VARIANT 17: note = R, S or K
  VARIANT 19: note = T, S, E, I, Sar, K, G or N-Me-Ile
  VARIANT 20..27: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..27: note = This region may encompass 1 to 8 amino acid residues
XXXXXXXCIXQXWXEHXCXXXXXXXXX

SEQ ID NO: 329 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y,1-Nal, 2-Nal or 1-Me-Trp
  VARIANT 12: note = E or D
  VARIANT 17: note = R, S or K
  VARIANT 19: note = T, I, S, E, K or Sar
  VARIANT 20..27: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..27: note = This region may encompass 1 to 8 amino acid residues
XXXXXXXCIXQXWGEHXCXXXXXXXXX

SEQ ID NO: 330 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 1-Nal, 2-Nal or 1-Me-Trp
  VARIANT 12: note = E or D
  VARIANT 19: note = T, S, E or Sar
  VARIANT 20..27: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..27: note = This region may encompass 1 to 8 amino acid residues
XXXXXXXCIXQXWGEHRCXXXXXXXXX

SEQ ID NO: 331 - protein, 25 aa, synthetic construct
  REGION 1..25: note = Description of Artificial Sequence: Synthetic peptide
  REGION 1..25: note = compstatin analogue
  VARIANT 1..6: note = A, E, G, L, K, F, P, S, T, W, Y, R, V, Sar, epsilonLys, gammaGlu, betaAsp, or betaAla, or a corresponding D form thereof, or Peg3, Peg4, 8-aminooctanoyl derivatives thereof or may be absent
  VARIANT 1..6: note = This region may encompass 1 to 6 amino acid residues
  VARIANT 7: note = Y or F
  DISULFID 8..18: note = Intrachain disulfide bond
  VARIANT 10: note = W, Y, or 1-Me-Trp
  VARIANT 12: note = E or D
  VARIANT 19: note = T, E or Sar
  VARIANT 20..25: note = A, E, G, L, K, F, P, S, T, W, Y, R, V, Sar, epsilonLys, gammaGlu, betaAsp, or betaAla, or a corresponding D form thereof or Peg3 or Peg4, 8-aminooctanoyl derivatives thereof or may be absent
  VARIANT 20..25: note = This region may encompass 1 to 6 amino acid residues
XXXXXXXCIXQXWGEHRCXXXXXXX

SEQ ID NO: 332 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, K covalently linked to a lipophilic group via its side chain, F, P, S, T, W, Y, R, V or Sar, a corresponding D form thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 2-Nal, 1-Nal or 1-Me-Trp
  VARIANT 12: note = E or D
  VARIANT 14: note = G or Sar
  VARIANT 17: note = R, S or K covalently linked to a lipophilic group via its side chain
  VARIANT 19: note = T, S, E, I, Sar, K, G or N-Me-Ile
  VARIANT 20..27: note = A, E, G, L, K, K covalently linked to a lipophilic group via its side chain, F, P, S, T, W, Y, R, V, Sar, epsilonLys, gammaGlu, betaAsp, betaAla, or a corresponding D form thereof or Peg 3 or Peg4, or 8-aminooctanoyl derivatives thereof or may be absent
  VARIANT 20..27: note = This region may encompass 1 to 8 amino acid residues
XXXXXXXCIXQXWXEHXCXXXXXXXXX

SEQ ID NO: 333 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, K covalently linked to a lipophilic group via its side chain, F, P, S, T, W, Y, R, V or Sar, a corresponding D form thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, Y, 1-Nal, 2-Nal or 1-Me-Trp
  VARIANT 12: note = E or D
  VARIANT 17: note = R, S or K covalently linked to a lipophilic group via its side chain
  VARIANT 19: note = T, I, S, E, K or Sar
  VARIANT 20..27: note = A, E, G, L, K, K covalently linked to a lipophilic group via its side chain F, P, S, T, W, Y, R, V, Sar, epsilonLys, gammaGlu, betaAsp, or betaAla, or a corresponding D form thereof or Peg3 or Peg4, or 8-aminooctanoyl derivatives thereof or may be absent
  VARIANT 20..27: note = This region may encompass 1 to 8 amino acid residues
XXXXXXXCIXQXWGEHXCXXXXXXXXX

SEQ ID NO: 334 - protein, 27 aa, synthetic construct
  REGION 1..27: note = Description of Artificial Sequence: Synthetic peptide
  REGION 1..27: note = compstatin analogue
  VARIANT 1..6: note = A, E, G, L, K, K covalently linked to a lipophilic group via its side chain, F, P, S, T, W, Y, R, V or Sar, a corresponding D form thereof or may be absent
  VARIANT 1..6: note = This region may encompass 1 to 6 amino acid residues
  VARIANT 7: note = I, Y, F or Sar
  DISULFID 8..18: note = Intrachain disulfide bond
  VARIANT 10: note = W, V, 1-Nal, 2-Nal or 1-Me-Trp
  VARIANT 12: note = E or D
  VARIANT 19: note = T, S, E or Sar
  VARIANT 20..27: note = A, E, G, L, K, K covalently linked to a lipophilic group via its side chain, F, P, S, T, W, Y, R, V, Sar, epsilonLys, gammaGlu, betaAsp, or betaAla, or a corresponding D form thereof or Peg3 or Peg4, or 8-aminooctanoyl derivatives thereof or may be absent
  VARIANT 20..27: note = This region may encompass 1 to 8 amino acid residues
XXXXXXXCIXQXWGEHRCXXXXXXXXX